(19) United States
(12) Patent Application Publication
(10) Pub. No.: US 2026/0315605 A1
(43) Pub. Date: Oct. 15, 2026

(54) PROTACS FOR ASK1 PROTEIN DEGRADATION: PREPARATION AND USE THEREOF

(71) Applicant: COUNCIL OF SCIENTIFIC AND INDUSTRIAL RESEARCH, New Delhi (IN)

(72) Inventors: Arindam TALUKDAR, Kolkata (IN); Himadri Sekhar SARKAR, Kolkata (IN); Partha CHAKRABARTI, Kolkata (IN); Israful HOQUE, Kolkata (IN); Abhishek SEN, Kolkata (IN); Uddipta Ghosh DASTIDAR, Kolkata (IN); Anindita DEY, Kolkata (IN)

(21) Appl. No.: 19/484,463

(22) PCT Filed: May 13, 2024
(86) PCT No.: PCT/IN2024/050526

§ 371 Date: Nov. 14, 2025

(30) Foreign Application Priority Data
May 18, 2023 (IN) 202311034982

(51) Int. Cl. A61K 47/55; A61K 47/54

(52) U.S. Cl. CPC A61K 47/55; A61K 47/545

(57) ABSTRACT

The preparation of substituted triazole-5-yl(pyridine-2-yl)benzamides compounds has structure I, in free form or an acceptable salt form for the targeted degradation of Apoptosis Signal-regulating Kinase 1 (ASK 1) as a potential therapeutic target for improves Nonalcoholic Fatty Liver Disease (NAFLD)/Nonalcoholic Steatohepatitis (NASH). Furthermore, small molecules where R₁, R₂, and R₃ are as defined in the description, are capable of targeted degradation of ASK1, which may ultimately be used to achieve improved human health in patients with hepatic fibrosis. Additionally, this represents the benchmark of this PROTAC technology for a potential therapeutic target protein ASK1 and will bring a breakthrough in NAFLD/NASH research.

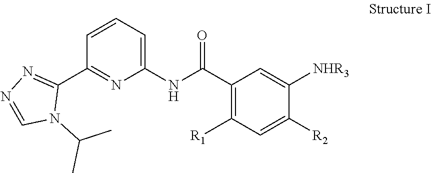

### FIELD OF THE INVENTION

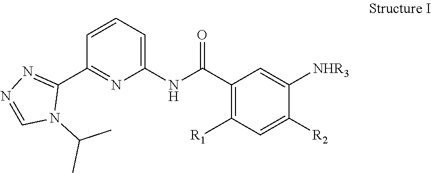

The present invention relates to the preparation of substituted triazole-5-yl(pyridine-2-yl)benzamides compounds, having structure I, in free form or an acceptable salt form for the targeted degradation of Apoptosis Signal-regulating Kinase 1 (ASK 1) as a potential therapeutic target for improves Nonalcoholic Fatty Liver Disease (NAFLD)/Nonalcoholic Steatohepatitis (NASH). More particularly, the invention relates to small molecules where R₁, R₂, and R₃ are as defined in the description, capable of targeted degradation of ASK1, which may be used to achieve improved human health in patients with hepatic fibrosis. The present disclosure represents the proteolysis targeting chimera (PROTAC) technology for a potential therapeutic target protein ASK1 and will bring a breakthrough in NAFLD/NASH research.

### BACKGROUND OF THE INVENTION

Apoptosis signal-regulating kinase 1 (ASK 1) is a mitogen-activated protein kinase kinase kinase (MAP3K) in the c-Jun N-terminal kinase (JNK) and p38 mitogen-activated protein kinase pathways, which play an important role in the elevation of hepatic inflammation and fibrosis (Xiang et al., J Hepatol. 64, 1365-1377, 2016). ASK1, an upstream regulator of P38 and JNK, has emerged as a promising drug target for limiting P38- and JNK-mediated diseases (Amos et al., J Cell Mol Med. 22, 4522-4533, 2018). Inhibition of ASK1 attenuates hepatocyte cell death, inflammation, and fibrogenesis and improves Nonalcoholic Fatty Liver Disease (NAFLD)/Nonalcoholic Steatohepatitis (NASH) (Wang et al., Nat Med. 23, 439-449, 2017). In 2020, Saroglitazar by Zydus Cadila, have been approved for the treatment of NASH in India (Gawriehat al., Hepatology. 1809-1824, 2021). Very recently, another small molecule drug selonsertib (GS-4997, developed by Gilead Sciences) showed promising anti-fibrotic activity by inhibiting the downregulation of the ASK1/MAPK pathway but failed in Phase III clinical trial of fibrosis improvement in NASH patients (Harrison et al., J Hepatol. 73, 26-39 (2020). Regardless of extensive clinical trials, licensed NAFLD remedy continues to be a challenge for the scientific fraternity. Therefore, the development of an alternative pharmacological approach to treat NAFLD/NASH is of utmost importance.

Inducing protein degradation by small molecules represents a promising approach to making “undruggable targets” druggable (Moon et al., Mol Cells 41, 933-942, 2018). Recently, Proteolysis Targeting Chimeras (PROTACs) represent a new class of promising therapeutic modalities that hijacks endogenous E3 ligases and the ubiquitin-proteasome system (UPS), leading to selective degradation of the target proteins (Békés et al., 21, 181-200, 2022). PROTACs induce selective polyubiquitination and degradation of the protein of interest (POI) by using bifunctional degradation inducers that link a target protein and an E3 ligase. PROTACs are potentially more advantageous to treat tumours compared to traditional small molecule inhibitors. PROTACs are heterobifunctional molecules made up of a target protein-binding ligand and an E3 ligase recruiting ligand connected by a suitable intervening linker. PROTACs cause proximity-dependent ubiquitination followed by proteasomal degradation of the target protein, effectively eliminating the protein's functions. Because of their unique mode of action, PROTACs have several advantages over small-molecule inhibitors, including catalytic activity and the ability to address the nonenzymatic functions of proteins (He et al., Front Cell Dev Biol. 9, 685106, 2021).). Hence, proteins previously described as undruggable can now be investigated as to whether they are degradable, offering new opportunities to develop treatments for diseases with currently unmet medical needs.

In 2022, ARV-110 (developed by Arvinas) and ARV-471 (co-developed by Arvinas and Pfizer), targeting the androgen receptor (AR) and estrogen receptor (ER), respectively, entered phase 2 clinical trial for treating prostate cancer and metastatic breast cancer (Li et al., Molecules 27, 8828, 2022). The outcome of the clinical trials signifies the translational potential of this newly developed PROTAC approach. There are no reports of this promising technology for selective degradation of potential therapeutic target protein ASK1.

Thus, keeping in view the drawbacks of the hitherto reported prior arts, herein, we report the design and multi-step organic synthesis platform for a set of PROTAC molecules. The design of our PROTACs contains a selonsertib fragment at one end and an E3 ligase ligand at the other end with different types of linkers in between. We choose the selonsertib fragment as a ligand for our target protein ASK1 (Ogier et al., J Mol Med., 98, 335-348, 2020). We have chosen thalidomide as a ligand for endogenous E3-ligase cereblon (CRBN), and a ligand for Von Hippel-Lindau (VHL) E3-ligase allowing us to degrade the target protein ASK1 selectively. We have already synthesized a large number of PROTAC degrader molecules, some of them showed excellent degradation efficiency within 1-100 nM, leading to fast, selective, efficient, and prolonged degradation of ASK1 in a proteasome-dependent manner in HepG2 and HEK293A cells. We have also validated in vitro cell-based assay protocol. The targeted degradation of ASK1 has great potential and may ultimately be used to achieve improved human health in patients with hepatic fibrosis. Thus, the present disclosure represents the benchmark of this PROTAC technology for a potential therapeutic target protein ASK1 and brings a breakthrough in NAFLD/NASH research.

### OBJECTIVES OF THE INVENTION

The main objective of the present invention is to provide triazole-5-yl(pyridine-2-yl)benzamides compounds having structure I.

Another objective of the present invention is to provide a process for the design, synthesis, and optimization of a series of triazole-5-yl(pyridine-2-yl)benzamides compounds having structure I.

Still another objective of the present invention is to synthesize different compounds having the structure I with a spacer of different linker lengths.

Yet another objective of the present invention is to probe other E3 ligases ligands, such as CRBN and VHL to improve the scope of targeted protein degradation.

Yet another objective of the present invention is to probe compounds having structure I that are capable of degrading ASK1 in a low nanomolar range leading to fast, selective, efficient, and prolonged degradation of ASK1 via E3-ligase ligands in a proteasome-dependent manner in HepG2 and HEK293 cells.

Still another objective of the present invention is to visualize the selective protein degradation by western blotting, fluorescence microscopy imaging, and proteomics.

Yet another objective of the present invention is to analyse and validate the structural basis of ASK1 degradation using CRBN by specific PROTAC through computational studies.

Still another objective of the present invention is to examine the ternary complex formation in cells by fluorescence microscopy imaging.

Yet another objective of the present invention is to apply the active PROTAC compounds in both proteasomal stress-induced in vitro assay and pre-clinical NASH models.

Still another objective of the present invention is to provide a composition comprising compounds of structure I for use in several clinical applications, including pharmaceutical agents and methods for treating disease conditions like NAFLD/NASH.

### SUMMARY OF THE INVENTION

The present invention relates to the triazole-5-yl(pyridine-2-yl)benzamides compounds represented by the following structure I or a pharmaceutically acceptable salt thereof:

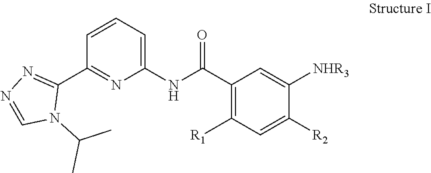

wherein
R₁ is independently selected from groups referred to as follows:

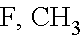

R₂ is independently selected from groups referred to as follows:

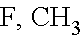

R₃ is independently selected from groups referred to as follows:

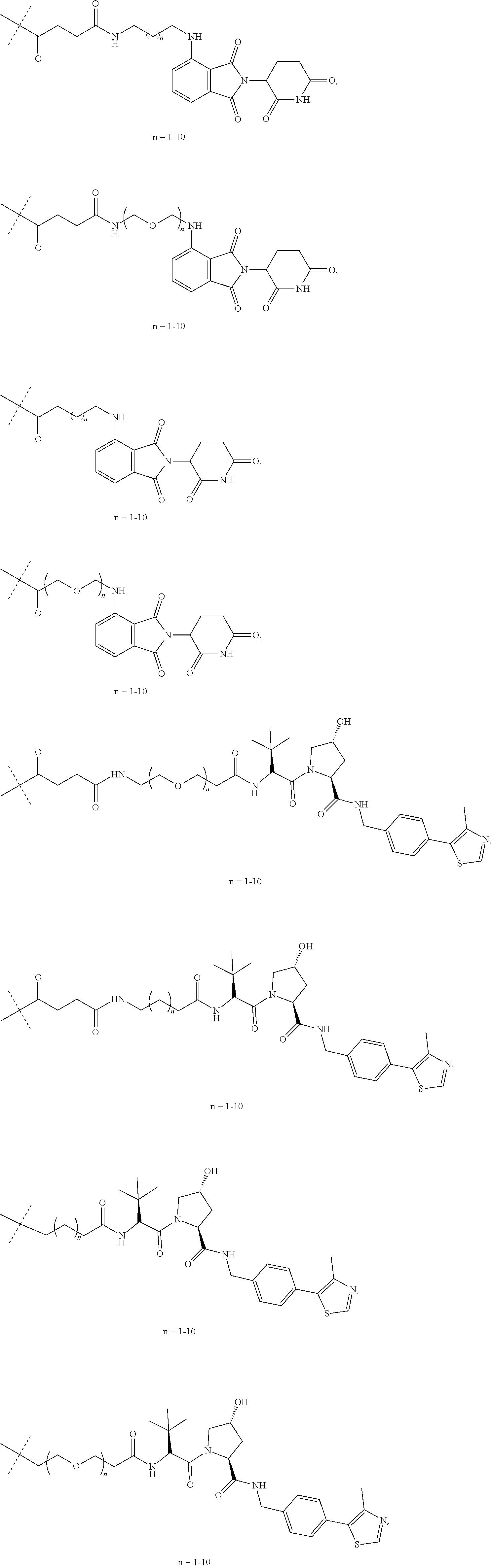

In an embodiment of the present invention, the triazole-5-yl(pyridine-2-yl)benzamides compounds having structure I is selected from the group consisting of:
N1-(2,4-difluoro-5-((6-(4-isopropyl-4H-1,2,4-triazol-3-yl)pyridin-2-yl)carbamoyl)phenyl)-N4-(2-(2-((2-(2,6-dioxopiperidin-3-yl)-1,3-dioxoisoindolin-4-yl)amino)ethoxy)ethyl)succinamide (16),
N1-(2,4-difluoro-5-((6-(4-isopropyl-4H-1,2,4-triazol-3-yl)pyridin-2-yl)carbamoyl)phenyl)-N4-(2-(2-(2-((2-(2,6-dioxopiperidin-3-yl)-1,3-dioxoisoindolin-4-yl)amino)ethoxy)ethoxy)ethyl)succinamide (19),
N1-(2,4-difluoro-5-((6-(4-isopropyl-4H-1,2,4-triazol-3-yl)pyridin-2-yl)carbamoyl)phenyl)-N4-(2-(2-(2-(2-((2-(2,6-dioxopiperidin-3-yl)-1,3-dioxoisoindolin-4-yl)amino)ethoxy)ethoxy)ethoxy)ethyl)succinamide (22),
N1-(2,4-difluoro-5-((6-(4-isopropyl-4H-1,2,4-triazol-3-yl)pyridin-2-yl)carbamoyl)phenyl)-N4-(14-((2-(2,6-dioxopiperidin-3-yl)-1,3-dioxoisoindolin-4-yl)amino)-3,6,9,12-tetraoxatetradecyl)succinamide (25),
N1-(2,4-difluoro-5-((6-(4-isopropyl-4H-1,2,4-triazol-3-yl)pyridin-2-yl)carbamoyl)phenyl)-N4-(17-((2-(2,6-dioxopiperidin-3-yl)-1,3-dioxoisoindolin-4-yl)amino)-3,6,9,12,15-pentaoxaheptadecyl)succinamide (28),
N1-(2,4-difluoro-5-((6-(4-isopropyl-4H-1,2,4-triazol-3-yl)pyridin-2-yl)carbamoyl)phenyl)-N4-(20-((2-(2,6-dioxopiperidin-3-yl)-1,3-dioxoisoindolin-4-yl)amino)-3,6,9,12,15,18-hexaoxaicosyl)succinamide (31),
N1-(2,4-difluoro-5-((6-(4-isopropyl-4H-1,2,4-triazol-3-yl)pyridin-2-yl)carbamoyl)phenyl)-N4-(5-((2-(2,6-dioxopiperidin-3-yl)-1,3-dioxoisoindolin-4-yl)amino)pentyl)succinamide (35),
5-(4-((2-(2,6-dioxopiperidin-3-yl)-1,3-dioxoisoindolin-4-yl)amino)butanamido)-2,4-difluoro-N-(6-(4-isopropyl-4H-1,2,4-triazol-3-yl)pyridin-2-yl)benzamide (38),
5-(5-((2-(2,6-dioxopiperidin-3-yl)-1,3-dioxoisoindolin-4-yl)amino)pentanamido)-2,4-difluoro-N-(6-(4-isopropyl-4H-1,2,4-triazol-3-yl)pyridin-2-yl)benzamide (41),
5-(6-((2-(2,6-dioxopiperidin-3-yl)-1,3-dioxoisoindolin-4-yl)amino)hexanamido)-2,4-difluoro-N-(6-(4-isopropyl-4H-1,2,4-triazol-3-yl)pyridin-2-yl)benzamide (44),
5-(7-((2-(2,6-dioxopiperidin-3-yl)-1,3-dioxoisoindolin-4-yl)amino)heptanamido)-2,4-difluoro-N-(6-(4-isopropyl-4H-1,2,4-triazol-3-yl)pyridin-2-yl)benzamide (47),
5-(8-((2-(2,6-dioxopiperidin-3-yl)-1,3-dioxoisoindolin-4-yl)amino)octanamido)-2,4-difluoro-N-(6-(4-isopropyl-4H-1,2,4-triazol-3-yl)pyridin-2-yl)benzamide (50),
5-(9-((2-(2,6-dioxopiperidin-3-yl)-1,3-dioxoisoindolin-4-yl)amino)nonanamido)-2,4-difluoro-N-(6-(4-isopropyl-4H-1,2,4-triazol-3-yl)pyridin-2-yl)benzamide (53),
5-(9-((2-(2,6-dioxopiperidin-3-yl)-1,3-dioxoisoindolin-4-yl)amino)nonanamido)-2,4-difluoro-N-(6-(4-isopropyl-4H-1,2,4-triazol-3-yl)pyridin-2-yl)benzamide (56),
N1-(2,4-difluoro-5-((6-(4-isopropyl-4H-1,2,4-triazol-3-yl)pyridin-2-yl)carbamoyl)phenyl)-N4-(2-(3-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)succinamide (61),
N1-(2,4-difluoro-5-((6-(4-isopropyl-4H-1,2,4-triazol-3-yl)pyridin-2-yl)carbamoyl)phenyl)-N4-(4-((2-(2,6-dioxopiperidin-3-yl)-1,3-dioxoisoindolin-4-yl)amino)butyl)succinamide (65),
N1-(2,4-difluoro-5-((6-(4-isopropyl-4H-1,2,4-triazol-3-yl)pyridin-2-yl)carbamoyl)phenyl)-N4-(6-((2-(2,6-dioxopiperidin-3-yl)-1,3-dioxoisoindolin-4-yl)amino)hexyl)succinamide (68),
N1-(2,4-difluoro-5-((6-(4-isopropyl-4H-1,2,4-triazol-3-yl)pyridin-2-yl)carbamoyl)phenyl)-N4-(7-((2-(2,6-dioxopiperidin-3-yl)-1,3-dioxoisoindolin-4-yl)amino)heptyl)succinamide (72),
N1-(2,4-difluoro-5-((6-(4-isopropyl-4H-1,2,4-triazol-3-yl)pyridin-2-yl)carbamoyl)phenyl)-N4-(8-((2-(2,6-dioxopiperidin-3-yl)-1,3-dioxoisoindolin-4-yl)amino)octyl)succinamide (75),
N1-(2,4-difluoro-5-((6-(4-isopropyl-4H-1,2,4-triazol-3-yl)pyridin-2-yl)carbamoyl)phenyl)-N4-(2-(2-(3-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)ethyl)succinamide (79),
N1-(2,4-difluoro-5-((6-(4-isopropyl-4H-1,2,4-triazol-3-yl)pyridin-2-yl)carbamoyl)phenyl)-N4-((R)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl)succinamide (83),
N1-(2,4-difluoro-5-((6-(4-isopropyl-4H-1,2,4-triazol-3-yl)pyridin-2-yl)carbamoyl)phenyl)-N4-((R)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl)succinamide (87),
N1-(2,4-difluoro-5-((6-(4-isopropyl-4H-1,2,4-triazol-3-yl)pyridin-2-yl)carbamoyl)phenyl)-N4-((R)-20-((2S,4R)-4-hydroxy-2-((4-(4-methylthiazol-5-yl)benzyl)carbamoyl)pyrrolidine-1-carbonyl)-21,21-dimethyl-18-oxo-3,6,9,12,15-pentaoxa-19-azadocosyl)succinamide (91),
N1-(2,4-difluoro-5-((6-(4-isopropyl-4H-1,2,4-triazol-3-yl)pyridin-2-yl)carbamoyl)phenyl)-N4-((R)-23-((2S,4R)-4-hydroxy-2-((4-(4-methylthiazol-5-yl)benzyl)carbamoyl)pyrrolidine-1-carbonyl)-24,24-dimethyl-21-oxo-3,6,9,12,15,18-hexaoxa-22-azapentacosyl)succinamide (95),
N1-(2,4-difluoro-5-((6-(4-isopropyl-4H-1,2,4-triazol-3-yl)pyridin-2-yl)carbamoyl)phenyl)-N4-(8-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctyl)succinamide (99),
N1-(2,4-difluoro-5-((6-(4-isopropyl-4H-1,2,4-triazol-3-yl)pyridin-2-yl)carbamoyl)phenyl)-N4-(8-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctyl)succinamide (103),
N1-(2,4-difluoro-5-((6-(4-isopropyl-4H-1,2,4-triazol-3-yl)pyridin-2-yl)carbamoyl)phenyl)-N4-(11-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecyl)succinamide (107),
N1-(2,4-difluoro-5-((6-(4-isopropyl-4H-1,2,4-triazol-3-yl)pyridin-2-yl)carbamoyl)phenyl)-N4-(14-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-14-oxotetradecyl)succinamide (111),
N1-(2,4-difluoro-5-((6-(4-isopropyl-4H-1,2,4-triazol-3-yl)pyridin-2-yl)carbamoyl)phenyl)-N4-(17-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-17-oxoheptadecyl)succinamide (115),
N1-(2,4-difluoro-5-((6-(4-isopropyl-4H-1,2,4-triazol-3-yl)pyridin-2-yl)carbamoyl)phenyl)-N4-(20-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-20-oxoicosyl)succinamide (119).

Yet another embodiment of the present invention provides a process for the preparation of triazole-5-yl(pyridine-2-yl)benzamides compounds having structure I, comprising the steps of:
(i) Compound 1 was taken in DICHLOROMETHANE and ACETONITRILE (1:1) & succinic anhydride and a catalytic amount of pyridine were added. The reaction mixture was then stirred at rt for 3.5 hours to produce compounds 2a, 2b, 2c, 2d, 2e, and 2f as a light yellowish liquid.
(ii) Compound 3 and compound 4 were taken in AcOH. NaOAc was added to the reaction mixture. The reaction mixture was then set to reflux for 12 hours to produce compound 5 as a white solid.
(iii) Compound 6 was taken in MeOH. Hydrazine hydrate was added to the reaction mixture and refluxed for 12 hours to produce Compound 7 as a yellowish solid.
(iv) Compound 7 was taken in 1,1-dimethoxy-N,N-dimethylmethanamine and was set to reflux for 12 hours to produce compound 8 as a deep brown liquid.
(v) Compound 8 was taken in a mixture of ACETONITRILE-AcOH (4:1). Propan-2-imine was then added to the reaction mixture and heated under reflux at 100° c. for 12 hours to produce compound 9 as off-white solid.
(vi) Compound 10 was taken in a DICHLOROMETHANE. Oxalyl chloride was added under the N2 atmosphere and stirred at rt for 2 hours. The solvent was then evaporated to dryness followed by the addition of the mixture of Compound 6 in pyridine. The final reaction mixture was then stirred at rt for another 2 hours to produce compound 12 as a yellow solid.
(vii) Compound 12 was taken in MeOH. SnCl₂ was added to the reaction mixture with a catalytic amount of HCl. The reaction mixture was then refluxed for 3 hours to produce compound 13 as a white powder.
(viii) Compound 13 was taken in DMF. HATU and DIPEA were then added to the reaction mixture. The reaction mixture was then stirred at rt for overnight to produce compounds 14, 17, 20, 23, 26, 29, 36, 39, 42, 45, 48, 51 and 54 as a colourless liquid.
(ix) Compound 14 was taken in a mixture of 4M HCl in dioxane in dioxane (1:1). The reaction mixture was then stirred at rt for 2 hours to produce compounds 15, 18, 21, 24, 27, 30, 37, 40, 43, 46, 49, 52, and 55 as a yellowish powder.
(x) Compound 15 was taken in DMF. DIPEA was added to the reaction mixture and set to reflux for 12 hours to produce compounds 16, 19, 22, 25, 28, 31, 38, 41, 44, 47, 50, 53, and 56 as a yellow solid.
(xi) Compound 5 was taken in DMSO. DIPEA was added to the reaction mixture and set to reflux for 12 hours to produce compound 32 as a yellow solid.
(xii) Compound 32 was taken in a mixture of 4M HCl in dioxane in DICHLOROMETHANE (1:1). The reaction mixture was then stirred at rt for 2 hours to produce compound 33 as a yellowish powder.
(xiii) Compound 33 was taken in DICHLOROMETHANE and ACETONITRILE (1:1) & succinic anhydride and a catalytic amount of pyridine were added. The reaction mixture was then stirred at rt for 3.5 hours to produce compound 34 as a light yellowish liquid.
(xiv) Compound 34 was taken in DMF. Compound 13, HATU and DIPEA were then added to the reaction mixture. The reaction mixture was then stirred at rt for overnight to produce compound 35 as a yellow solid.
(xv) Compound 57 was taken in DMF. Compound 3-(2-((tert-butoxycarbonyl)amino)ethoxy)propanoic acid, HATU and DIPEA were then added to the reaction mixture. The reaction mixture was then stirred at rt for overnight to produce compound 58 as a colourless liquid.
(xvi) Compound 58 was taken in a mixture of 4M HCl in dioxane in dioxane (1:1). The reaction mixture was then stirred at rt for 2 hours to produce compound 59 as a white powder.
(xvii) Compound 59 was taken in DICHLOROMETHANE and ACETONITRILE (1:1) & succinic anhydride and a catalytic amount of pyridine were added. The reaction mixture was then stirred at rt for 3.5 hours to produce compound 60 as a white solid.
(xviii) Compound 60 was taken in DMF. Compound 13, HATU and DIPEA were then added to the reaction mixture. The reaction mixture was then stirred at rt for overnight to produce compound 61 as a white solid.

### BRIEF DESCRIPTION OF DRAWINGS

The invention has other advantages and features which will be more readily apparent from the following detailed description of the invention and the appended claims, when taken in conjunction with the accompanying drawings, in which:

FIG. 1 Degradation of ASK1 in HepG2 and HEK293A cells. Mol. wt. of ASK1: 155 kDa; Mol. wt. of Actin: 42 kDa; 8% Polyacrylamide gel for SDS-PAGE; Sample amount: 30-40 μL in each well; Primary antibody: Anti-ASK1 (1:1000) and Anti-Actin (1:5000); Secondary antibody: Anti Mouse HRP (1:5000); and Anti Rabbit (1:1000) Detection method: Chemi-Luminescence; Instrument: ChemDoc LAS 500; Exposure: Auto exposure (30 secs-1 min).

FIG. 2 Degradation of ASK1 in 4 (a), 8 (b), and 12 hrs (c) of PROTAC-16 treatment.

FIG. 3 Rescue of ASK1 degradation by PROTAC-16 in HepG2 cells by proteasomal inhibitor MG132.

FIG. 4 Optimization of the HepG2 cell numbers for the effective degradation of ASK1 with the PROTAC-16.

FIG. 5 Flow chart summarizing the experimental protocol for the overall degradation experiments.

FIG. 6 Complex of CRBN-ASK1-DDB1 proteins with docked PROTAC-16 and its binding interactions in zoomed view.

FIG. 7 Percentage Fold change of the ASK1 expression in HEK293A cells with increasing concentration of PROTAC-16. PROTAC-16 degrades target protein ASK1 dose-dependently. 10 nM of the PROTAC-16 degrades over 80% of intracellular ASK1. At high concentrations, PROTAC-16 can saturate binding sites on either the POI ASK1 or the E3 ligase CRBN, without forming the required ternary complex. This phenomenon is called the Hook effect, which is the signature bell-shaped concentration dependence of activity for PROTAC degraders.

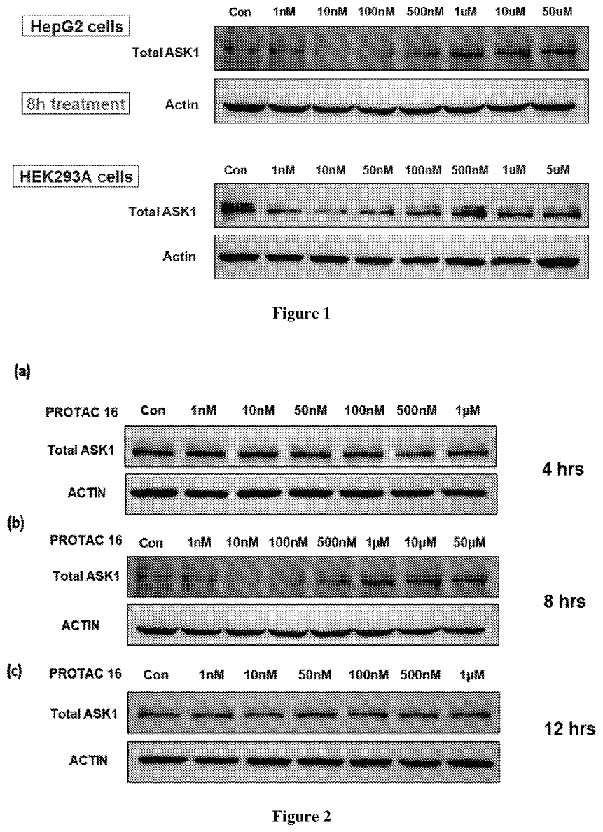

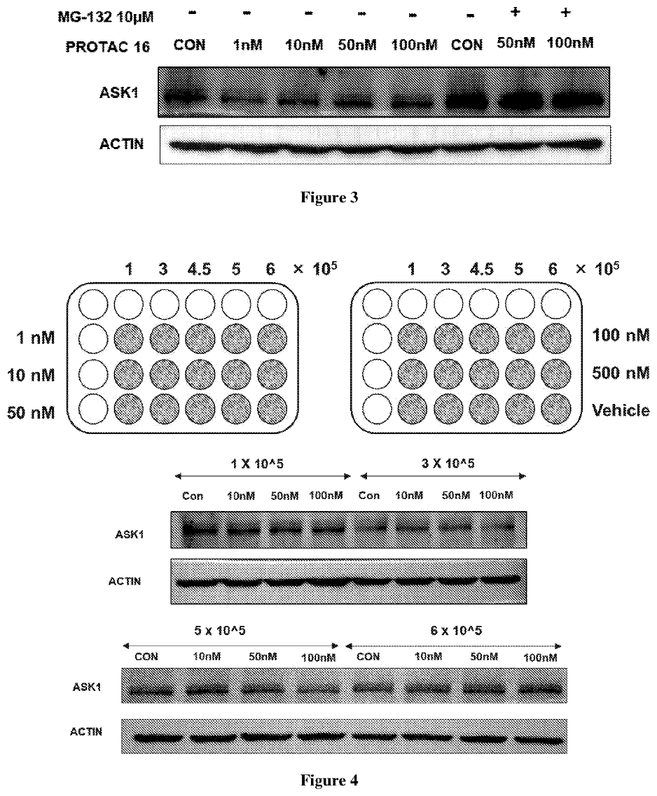

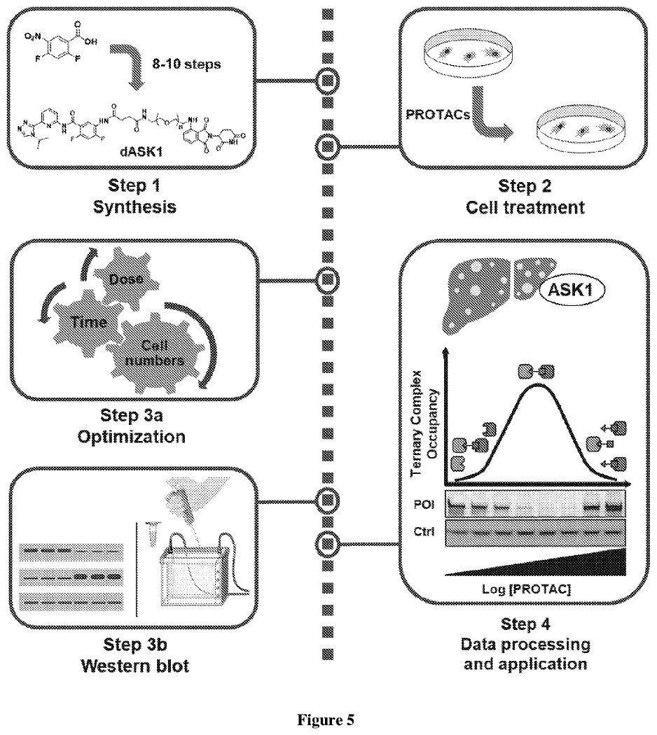

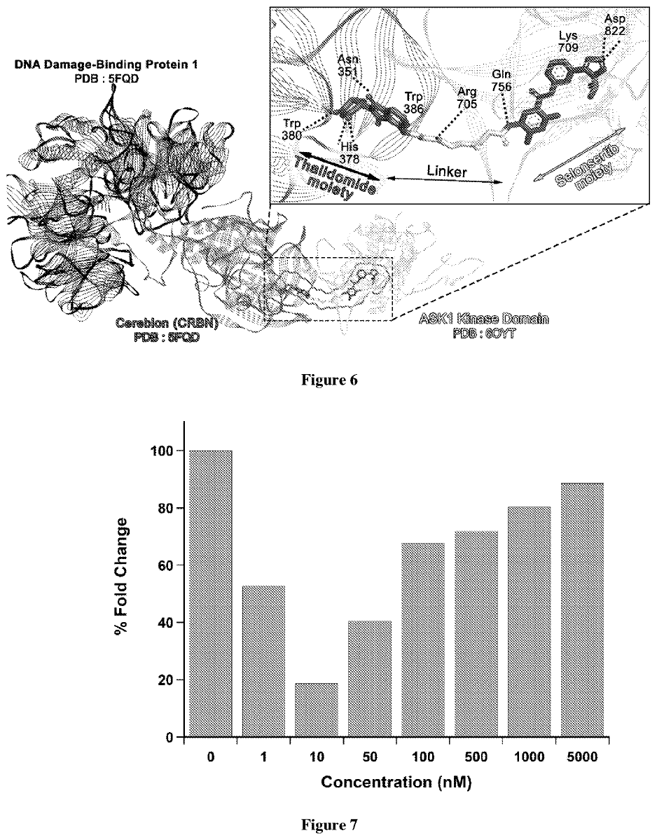

### DETAILED DESCRIPTION OF THE INVENTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope. Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein. In line with the above objectives, the present invention relates to the triazole-5-yl(pyridine-2-yl)benzamides compounds represented by the following structure I or a pharmaceutically acceptable salt thereof:

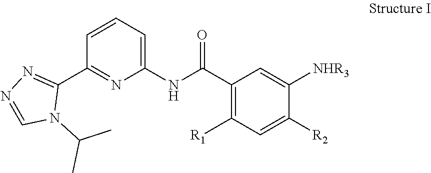

wherein
R₁ is independently selected from groups referred to as follows:

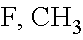

R₂ is independently selected from groups referred to as follows:

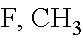

R₃ is independently selected from groups referred to as follows:

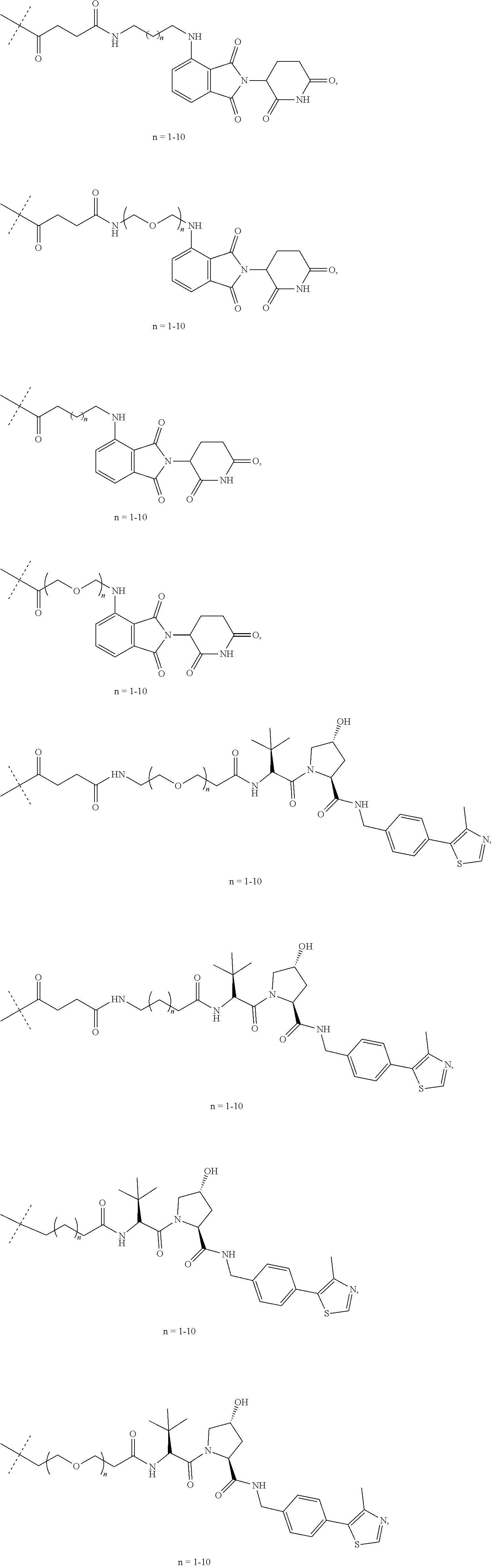

The triazole-5-yl(pyridine-2-yl)benzamides compounds of the present invention has the structure of Structure I as depicted in the table below:

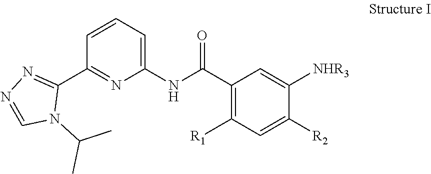

TABLE 1
Structure of the compounds disclosed (Table 1)

| | Compound | | |
|---|---|---|---|
| Entry | number | Structure | IUPAC Name |
| 1 | 16 | 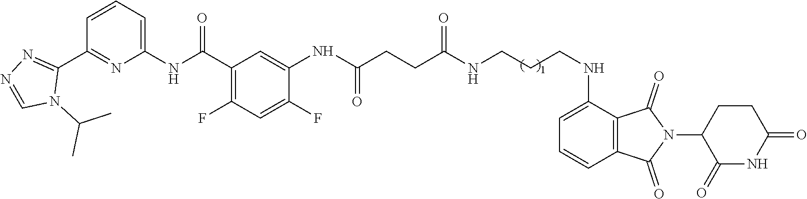 | N1-(2,4-difluoro-5-((6-(4-isopropyl- 4H-1,2,4-triazol-3-yl)pyridin-2- yl)carbamoyl)phenyl)-N4-(2-(2-((2- (2,6-dioxopiperidin-3-yl)-1,3- dioxoisoindolin-4- yl)amino)ethoxy)ethyl)succinamide |
| 2 | 19 | 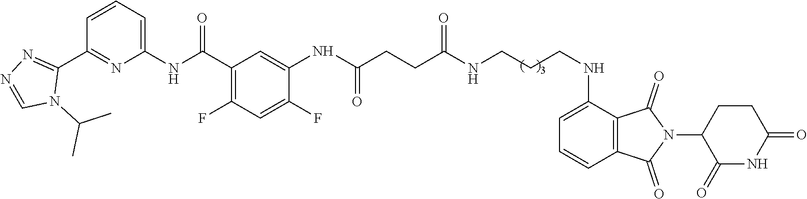 | N1-(2,4-difluoro-5-((6-(4-isopropyl- 4H-1,2,4-triazol-3-yl)pyridin-2- yl)carbamoyl)phenyl)-N4-(2-(2-(2-((2- (2,6-dioxopiperidin-3-yl)-1,3- dioxoisoindolin-4- yl)amino)ethoxy)ethoxy)ethyl) succinamide |
| 3 | 22 | 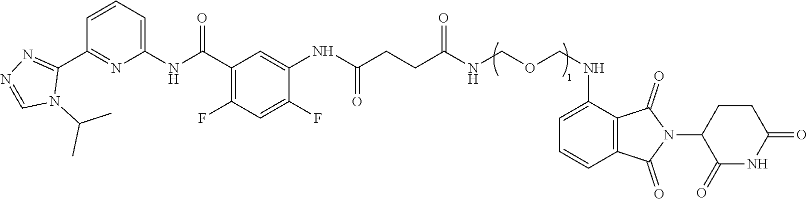 | N1-(2,4-difluoro-5-((6-(4-isopropyl- 4H-1,2,4-triazol-3-yl)pyridin-2- yl)carbamoyl)phenyl)-N4-(2-(2-(2-(2- ((2-(2,6-dioxopiperidin-3-yl)-1,3- dioxoisoindolin-4- yl)amino)ethoxy)ethoxy)ethoxy)ethyl) succinamide |
| 4 | 25 | 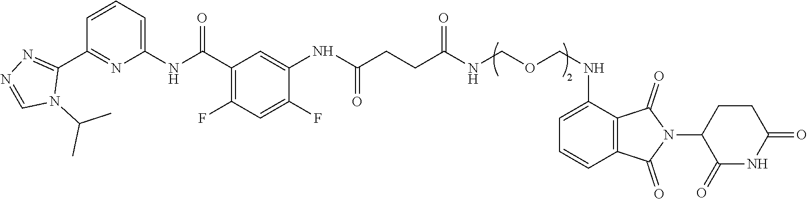 | N1-(2,4-difluoro-5-((6-(4-isopropyl- 4H-1,2,4-triazol-3-yl)pyridin-2- yl)carbamoyl)phenyl)-N4-(14-((2- (2,6-dioxopiperidin-3-yl)-1,3- dioxoisoindolin-4-yl)amino)-3,6,9,12- tetraoxatetradecyl)succinamide |
| 5 | 28 | 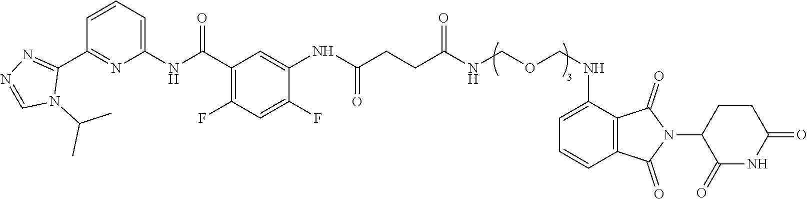 | N1-(2,4-difluoro-5-((6-(4-isopropyl- 4H-1,2,4-triazol-3-yl)pyridin-2- yl)carbamoyl)phenyl)-N4-(17-((2- (2,6-dioxopiperidin-3-yl)-1,3- dioxoisoindolin-4-yl)amino)- 3,6,9,12,15- pentaoxaheptadecyl)succinamide |
| 6 | 31 | 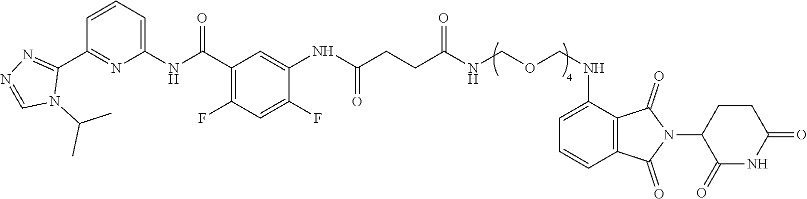 | N1-(2,4-difluoro-5-((6-(4-isopropyl- 4H-1,2,4-triazol-3-yl)pyridin-2- yl)carbamoyl)phenyl)-N4-(20-((2- (2,6-dioxopiperidin-3-yl)-1,3- dioxoisoindolin-4-yl)amino)- 3,6,9,12,15,18- hexaoxaicosyl)succinamide |
| 7 | 35 | 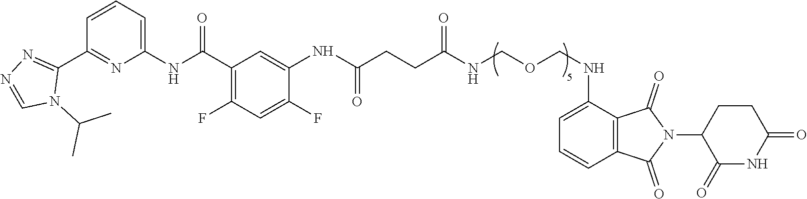 | N1-(2,4-difluoro-5-((6-(4-isopropyl- 4H-1,2,4-triazol-3-yl)pyridin-2- yl)carbamoyl)phenyl)-N4-(5-((2-(2,6- dioxopiperidin-3-yl)-1,3- dioxoisoindolin-4- yl)amino)pentyl)succinamide |
| 8 | 38 | 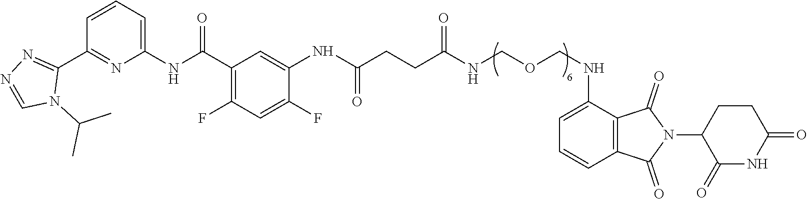 | 5-(4-((2-(2,6-dioxopiperidin-3-yl)-1,3- dioxoisoindolin-4- yl)amino)butanamido)-2,4-difluoro-N- (6-(4-isopropyl-4H-1,2,4-triazol-3- yl)pyridin-2-yl)benzamide |
| 9 | 41 | 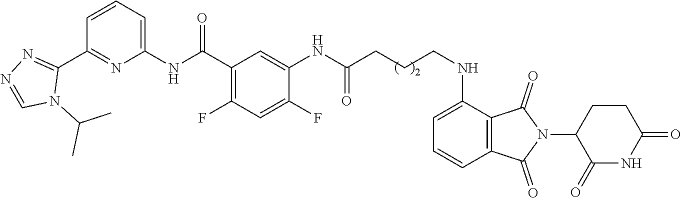 | 5-(5-((2-(2,6-dioxopiperidin-3-yl)-1,3- dioxoisoindolin-4- yl)amino)pentanamido)-2,4-difluoro- N-(6-(4-isopropyl-4H-1,2,4-triazol-3- yl)pyridin-2-yl)benzamide |
| 10 | 44 | 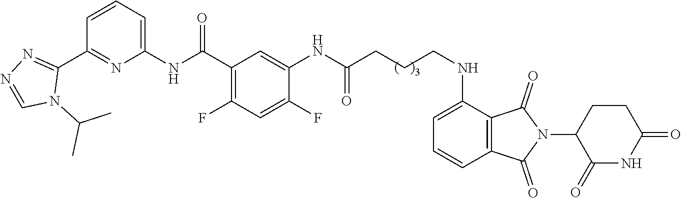 | 5-(6-((2-(2,6-dioxopiperidin-3-yl)-1,3- dioxoisoindolin-4- yl)amino)hexanamido)-2,4-difluoro- N-(6-(4-isopropyl-4H-1,2,4-triazol-3- yl)pyridin-2-yl)benzamide |
| 11 | 47 | 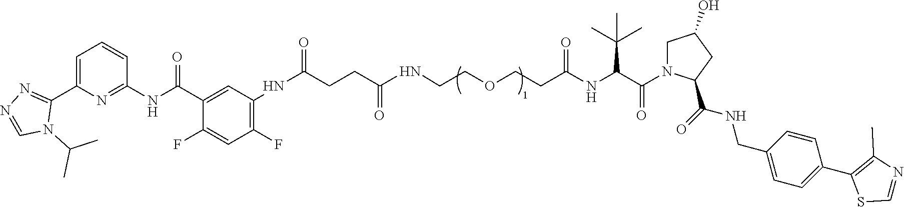 | 5-(7-((2-(2,6-dioxopiperidin-3-yl)-1,3- dioxoisoindolin-4- yl)amino)heptanamido)-2,4-difluoro- N-(6-(4-isopropyl-4H-1,2,4-triazol-3- yl)pyridin-2-yl)benzamide |
| 12 | 50 | 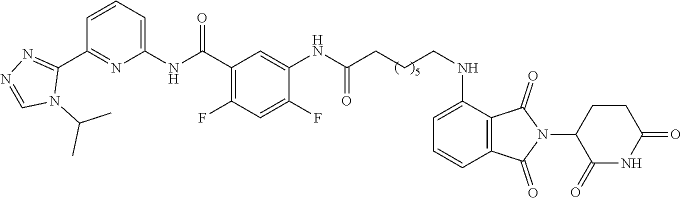 | 5-(8-((2-(2,6-dioxopiperidin-3-yl)-1,3- dioxoisoindolin-4- yl)amino)octanamido)-2,4-difluoro-N- (6-(4-isopropyl-4H-1,2,4-triazol-3- yl)pyridin-2-yl)benzamide |
| 13 | 53 | 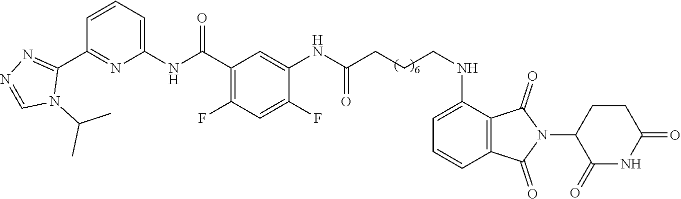 | 5-(9-((2-(2,6-dioxopiperidin-3-yl)-1,3- dioxoisoindolin-4- yl)amino)nonanamido)-2,4-difluoro- N-(6-(4-isopropyl-4H-1,2,4-triazol-3- yl)pyridin-2-yl)benzamide |
| 14 | 56 | 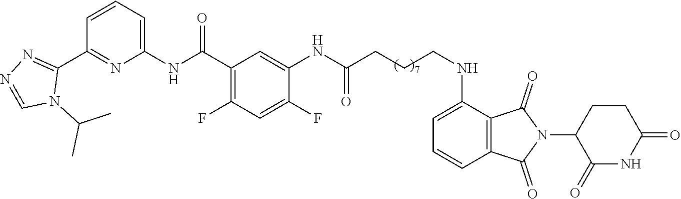 | 5-(9-((2-(2,6-dioxopiperidin-3-yl)-1,3- dioxoisoindolin-4- yl)amino)nonanamido)-2,4-difluoro- N-(6-(4-isopropyl-4H-1,2,4-triazol-3- yl)pyridin-2-yl)benzamide (56) |
| 15 | 61 | 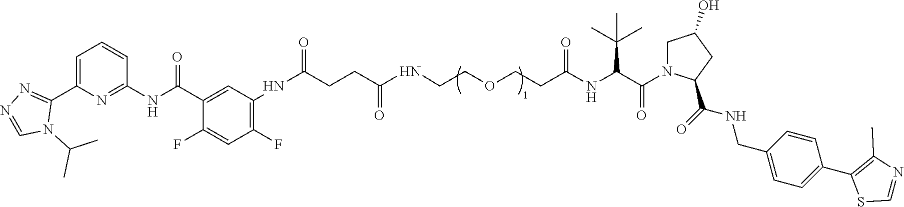 | N1-(2,4-difluoro-5-((6-(4-isopropyl- 4H-1,2,4-triazol-3-yl)pyridin-2- yl)carbamoyl)phenyl)-N4-(2-(3-(((R)- 1-((2S,4R)-4-hydroxy-2-((4-(4- methylthiazol-5-yl)benzyl)carbamoyl) pyrrolidin-1-yl)-3,3-dimethyl-1- oxobutan-2-yl)amino)-3- oxopropoxy)ethyl)succinamide (61) |
| 16 | 65 | 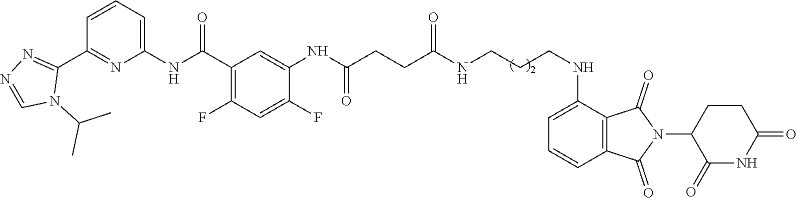 | N1-(2,4-difluoro-5-((6-(4-isopropyl- 4H-1,2,4-triazol-3-yl)pyridin-2- yl)carbamoyl)phenyl)-N4-(4-((2-(2,6- dioxopiperidin-3-yl)-1,3- dioxoisoindolin-4- yl)amino)butyl)succinamide |
| 17 | 68 | 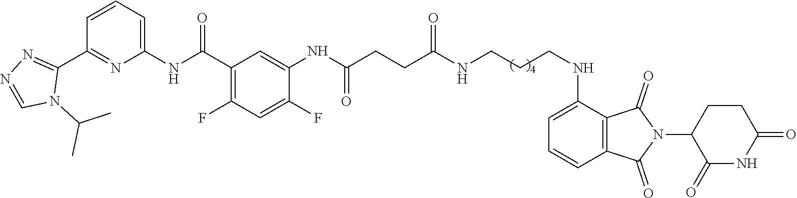 | N1-(2,4-difluoro-5-((6-(4-isopropyl- 4H-1,2,4-triazol-3-yl)pyridin-2- yl)carbamoyl)phenyl)-N4-(6-((2-(2,6- dioxopiperidin-3-yl)-1,3- dioxoisoindolin-4- yl)amino)hexyl)succinamide |
| 18 | 72 | 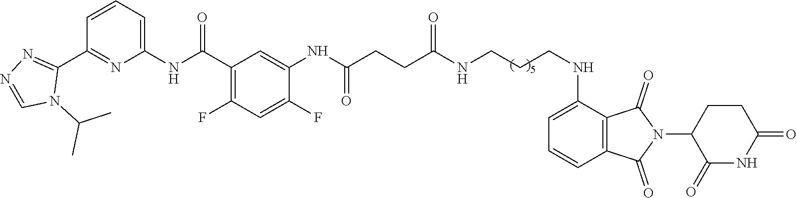 | N1-(2,4-difluoro-5-((6-(4-isopropyl- 4H-1,2,4-triazol-3-yl)pyridin-2- yl)carbamoyl)phenyl)-N4-(7-((2-(2,6- dioxopiperidin-3-yl)-1,3- dioxoisoindolin-4- yl)amino)heptyl)succinamide |
| 19 | 75 | 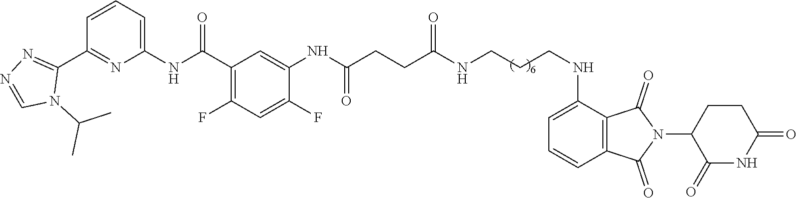 | N1-(2,4-difluoro-5-((6-(4-isopropyl- 4H-1,2,4-triazol-3-yl)pyridin-2- yl)carbamoyl)phenyl)-N4-(8-((2-(2,6- dioxopiperidin-3-yl)-1,3- dioxoisoindolin-4- yl)amino)octyl)succinamide |
| 20 | 79 | 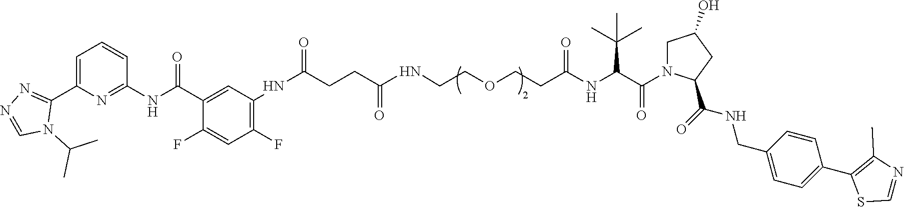 | N1-(2,4-difluoro-5-((6-(4-isopropyl- 4H-1,2,4-triazol-3-yl)pyridin-2- yl)carbamoyl)phenyl)-N4-(2-(2-(3- (((R)-1-((2S,4R)-4-hydroxy-2-((4-(4- methylthiazol-5- yl)benzyl)carbamoyl)pyrrolidin-1-yl)- 3,3-dimethyl-1-oxobutan-2-yl)amino)- 3-oxopropoxy)ethoxy) ethyl)succinamide |
| 21 | 83 | 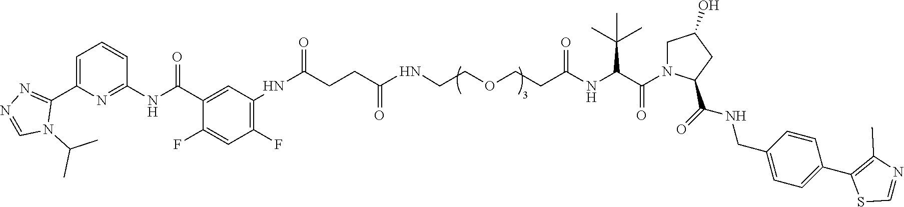 | N1-(2,4-difluoro-5-((6-(4-isopropyl- 4H-1,2,4-triazol-3-yl)pyridin-2- yl)carbamoyl)phenyl)-N4-((R)-14- ((2S,4R)-4-hydroxy-2-((4-(4- methylthiazol-5- yl)benzyl)carbamoyl)pyrrolidine-1- carbonyl)-15,15-dimethyl-12-oxo- 3,6,9-trioxa-13- azahexadecyl)succinamide |
| 22 | 87 | 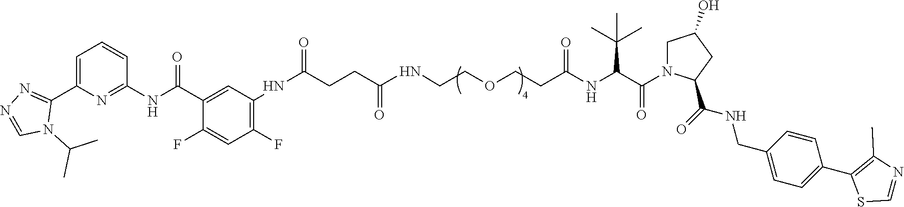 | N1-(2,4-difluoro-5-((6-(4-isopropyl- 4H-1,2,4-triazol-3-yl)pyridin-2- yl)carbamoyl)phenyl)-N4-((R)-17- ((2S,4R)-4-hydroxy-2-((4-(4- methylthiazol-5- yl)benzyl)carbamoyl)pyrrolidine-1- carbonyl)-18,18-dimethyl-15-oxo- 3,6,9,12-tetraoxa-16- azanonadecyl)succinamide |
| 23 | 91 | 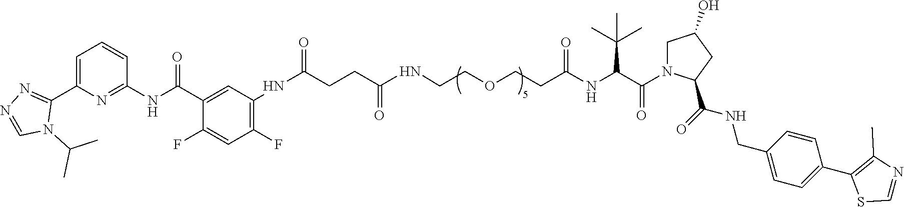 | N1-(2,4-difluoro-5-((6-(4-isopropyl- 4H-1,2,4-triazol-3-yl)pyridin-2- yl)carbamoyl)phenyl)-N4-((R)-20- ((2S,4R)-4-hydroxy-2-((4-(4- methylthiazol-5- yl)benzyl)carbamoyl)pyrrolidine-1- carbonyl)-21,21-dimethyl-18-oxo- 3,6,9,12,15-pentaoxa-19- azadocosyl)succinamide |
| 24 | 95 | 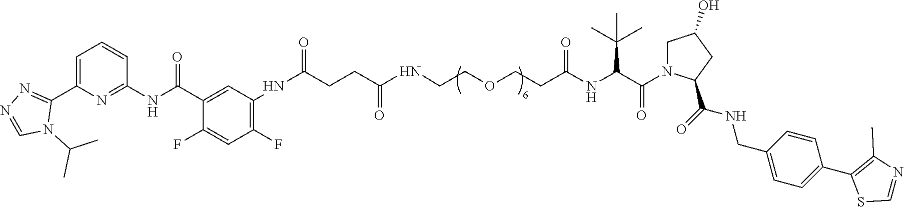 | N1-(2,4-difluoro-5-((6-(4-isopropyl- 4H-1,2,4-triazol-3-yl)pyridin-2- yl)carbamoyl)phenyl)-N4-((R)-23- ((2S,4R)-4-hydroxy-2-((4-(4- methylthiazol-5- yl)benzyl)carbamoyl)pyrrolidine-1- carbonyl)-24,24-dimethyl-21-oxo- 3,6,9,12,15,18-hexaoxa-22- azapentacosyl)succinamide |
| 25 | 99 | 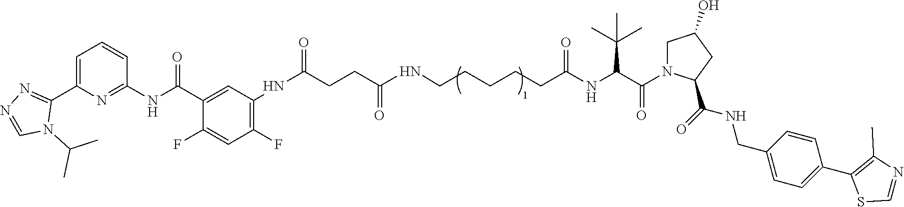 | N1-(2,4-difluoro-5-((6-(4-isopropyl- 4H-1,2,4-triazol-3-yl)pyridin-2- yl)carbamoyl)phenyl)-N4-(6-(((R)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)amino)- 6-oxohexyl)succinamide |
| 26 | 103 | 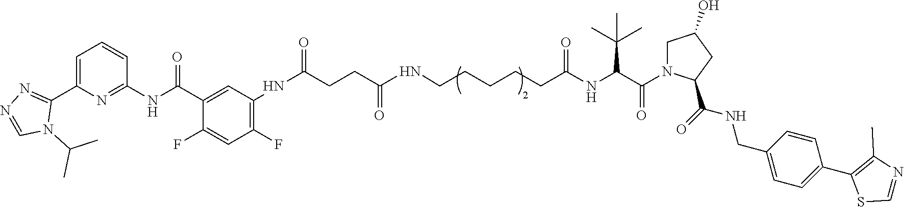 | N1-(2,4-difluoro-5-((6-(4-isopropyl- 4H-1,2,4-triazol-3-yl)pyridin-2- yl)carbamoyl)phenyl)-N4-(8-(((R)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)amino)- 8-oxooctyl)succinamide |
| 27 | 107 | 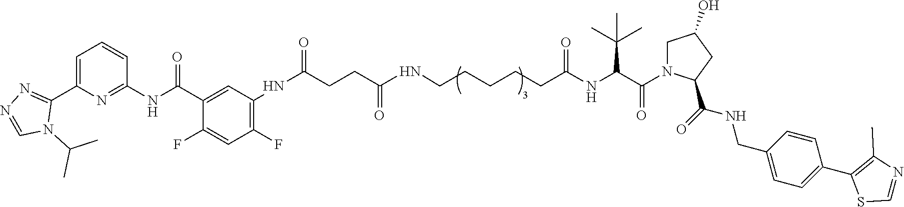 | N1-(2,4-difluoro-5-((6-(4-isopropyl- 4H-1,2,4-triazol-3-yl)pyridin-2- yl)carbamoyl)phenyl)-N4-(11-(((R)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)amino)- 11-oxoundecyl)succinamide |
| 28 | 111 | 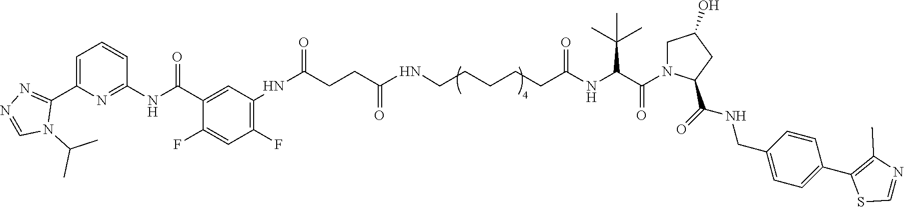 | N1-(2,4-difluoro-5-((6-(4-isopropyl- 4H-1,2,4-triazol-3-yl)pyridin-2- yl)carbamoyl)phenyl)-N4-(14-(((R)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)amino)- 14-oxotetradecyl)succinamide |
| 29 | 115 | 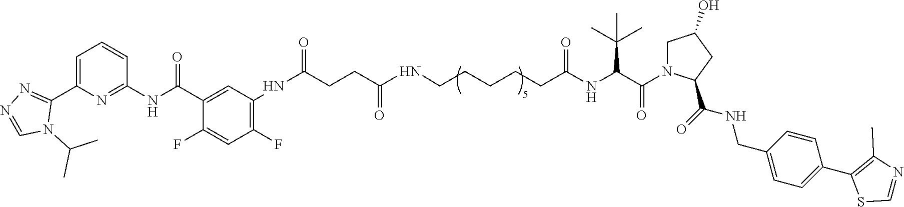 | N1-(2,4-difluoro-5-((6-(4-isopropyl- 4H-1,2,4-triazol-3-yl)pyridin-2- yl)carbamoyl)phenyl)-N4-(17-(((R)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)amino)- 17-oxoheptadecyl)succinamide |
| 30 | 119 | 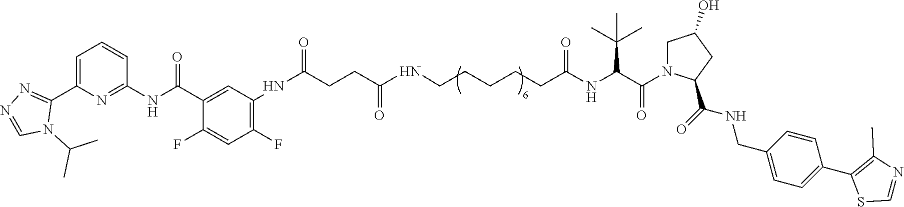 | N1-(2,4-difluoro-5-((6-(4-isopropyl- 4H-1,2,4-triazol-3-yl)pyridin-2- yl)carbamoyl)phenyl)-N4-(20-(((R)-1- ((2S,4R)-4-hydroxy-2-((4-(4- methylthiazol-5- yl)benzyl)carbamoyl)pyrrolidin-1-yl)- 3,3-dimethyl-1-oxobutan-2-yl)amino)- 20-oxoicosyl)succinamide |

### General Process of Preparation:

(i) Compound 1 was taken in dichloromethane and acetonitrile (1:1) & succinic anhydride and a catalytic amount of pyridine were added. The reaction mixture was then stirred at rt for 3.5 hours to produce compounds 2a, 2b, 2c, 2d, 2e, and 2f as a light yellowish liquid.
(ii) Compound 3 and compound 4 were taken in acetic acid. Sodium acetate was added to the reaction mixture. The reaction mixture was then set to reflux for 12 hours to produce compound 5 as a white solid.
(iii) Compound 6 was taken in methanol. Hydrazine hydrate was added to the reaction mixture and refluxed for 12 hours to produce Compound 7 as a yellowish solid.
(iv) Compound 7 was taken in 1,1-dimethoxy-N,N-dimethylmethanamine and was set to reflux for 12 hours to produce compound 8 as a deep brown liquid.
(v) Compound 8 was taken in a mixture of acetonitrile-acetic acid (4:1). Propan-2-imine was then added to the reaction mixture and heated under reflux at 100° c. for 12 hours to produce compound 9 as an off-white solid.
(vi) Compound 10 was taken in a dichloromethane. Oxalyl chloride was added under the N2 atmosphere and stirred at rt for 2 hours. The solvent was then evaporated to dryness followed by the addition of the mixture of compound 6 in pyridine. The final reaction mixture was then stirred at rt for another 2 hours to produce compound 12 as a yellow solid.
(vii) Compound 12 was taken in methanol. Stannous chloride was added to the reaction mixture with a catalytic amount of HCl. The reaction mixture was then refluxed for 3 hours to produce compound 13 as a white powder.
(viii) Compound 13 was taken in N,N-dimethylformamide. HATU and N,N-diisopropylethylamine were then added to the reaction mixture. The reaction mixture was then stirred at rt for overnight to produce compounds 14, 17, 20, 23, 26, 29, 36, 39, 42, 45, 48, 51 and 54 as a colourless liquid.
(ix) Compound 14 was taken in a mixture of 4M HCl in dioxane in dioxane (1:1). The reaction mixture was then stirred at rt for 2 hours to produce compounds 15, 18, 21, 24, 27, 30, 37, 40, 43, 46, 49, 52, and 55 as a yellowish powder.
(x) Compound 15 was taken in N,N-dimethylformamide. N,N-diisopropylethylamine was added to the reaction mixture and set to reflux for 12 hours to produce compounds 16, 19, 22, 25, 28, 31, 38, 41, 44, 47, 50, 53, and 56 as a yellow solid.
(xi) Compound 5 was taken in dimethyl sulfoxide. N,N-diisopropylethylamine was added to the reaction mixture and set to reflux for 12 hours to produce compound 32 as a yellow solid.
(xii) Compound 32 was taken in a mixture of 4M HCl in dioxane in dichloromethane (1:1). The reaction mixture was then stirred at rt for 2 hours to produce compound 33 as a yellowish powder.
(xiii) Compound 33 was taken in dichloromethane and acetonitrile (1:1) & succinic anhydride and a catalytic amount of pyridine were added. The reaction mixture was then stirred at rt for 3.5 hours to produce compound 34 as a light yellowish liquid.
(xiv) Compound 34 was taken in N,N-dimethylformamide. Compound 13, HATU and N,N-diisopropylethylamine were then added to the reaction mixture. The reaction mixture was then stirred at rt for overnight to produce compound 35 as a yellow solid.
(xv) Compound 57 was taken in N,N-dimethylformamide. Compound 3-(2-((tert-butoxycarbonyl)amino)ethoxy)propanoic acid, HATU and N,N-diisopropylethylamine were then added to the reaction mixture. The reaction mixture was then stirred at rt for overnight to produce compound 58 as a colourless liquid.
(xvi) Compound 58 was taken in a mixture of 4M HCl in dioxane in dioxane (1:1). The reaction mixture was then stirred at rt for 2 hours to produce compound 59 as a white powder.
(xvii) Compound 59 was taken in dichloromethane and acetonitrile (1:1) & succinic anhydride and a catalytic amount of pyridine were added. The reaction mixture was then stirred at rt for 3.5 hours to produce compound 60 as a white solid.
(xviii) Compound 60 was taken in N,N-dimethylformamide. Compound 13, HATU and N,N-diisopropylethylamine were then added to the reaction mixture. The reaction mixture was then stirred at rt for overnight to produce compound 61 as a white solid.

Abbreviations:

| | |
|---|---|
| DMF | N,N-dimethylformamide |
| ACETONITRILE | Acetonitrile |
| MeOH | Methanol |
| NaOAc | Sodium acetate |
| DIPEA | N,N-Diisopropylethylamine |
| THF | Tetrahydrofuran |
| SnCl₂ | Stannous chloride |
| Ar | Argon |
| AcOH | Acetic Acid |
| HATU | 1-[Bis(dimethylamino)methylene]-1H-1,2,3- |
| | triazolo[4,5-b]pyridinium 3-oxidehexafluorophosphate |

Provided below are the schemes for preparing the compounds disclosed in the present application.

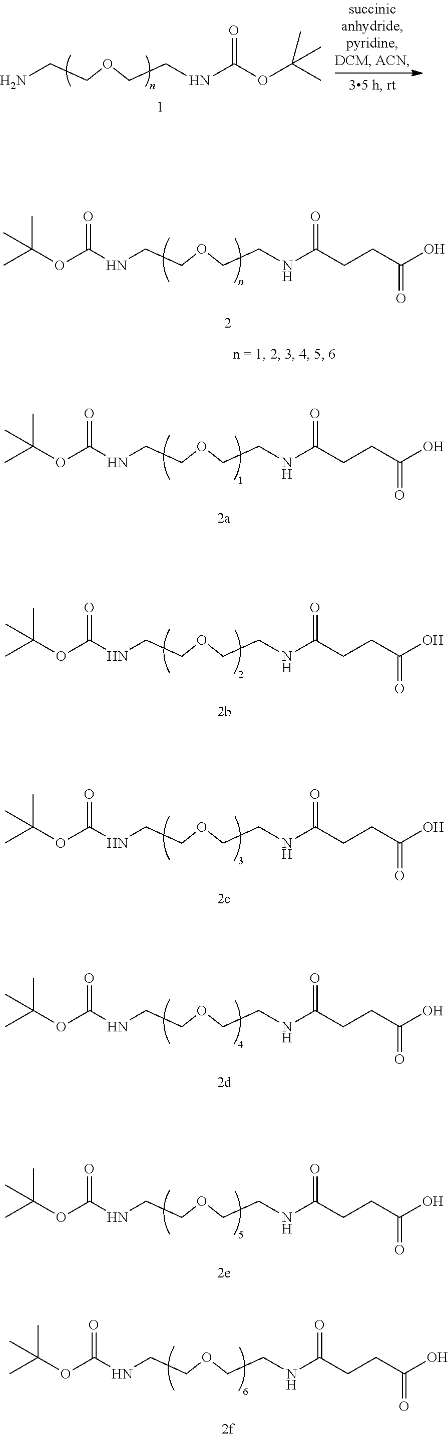

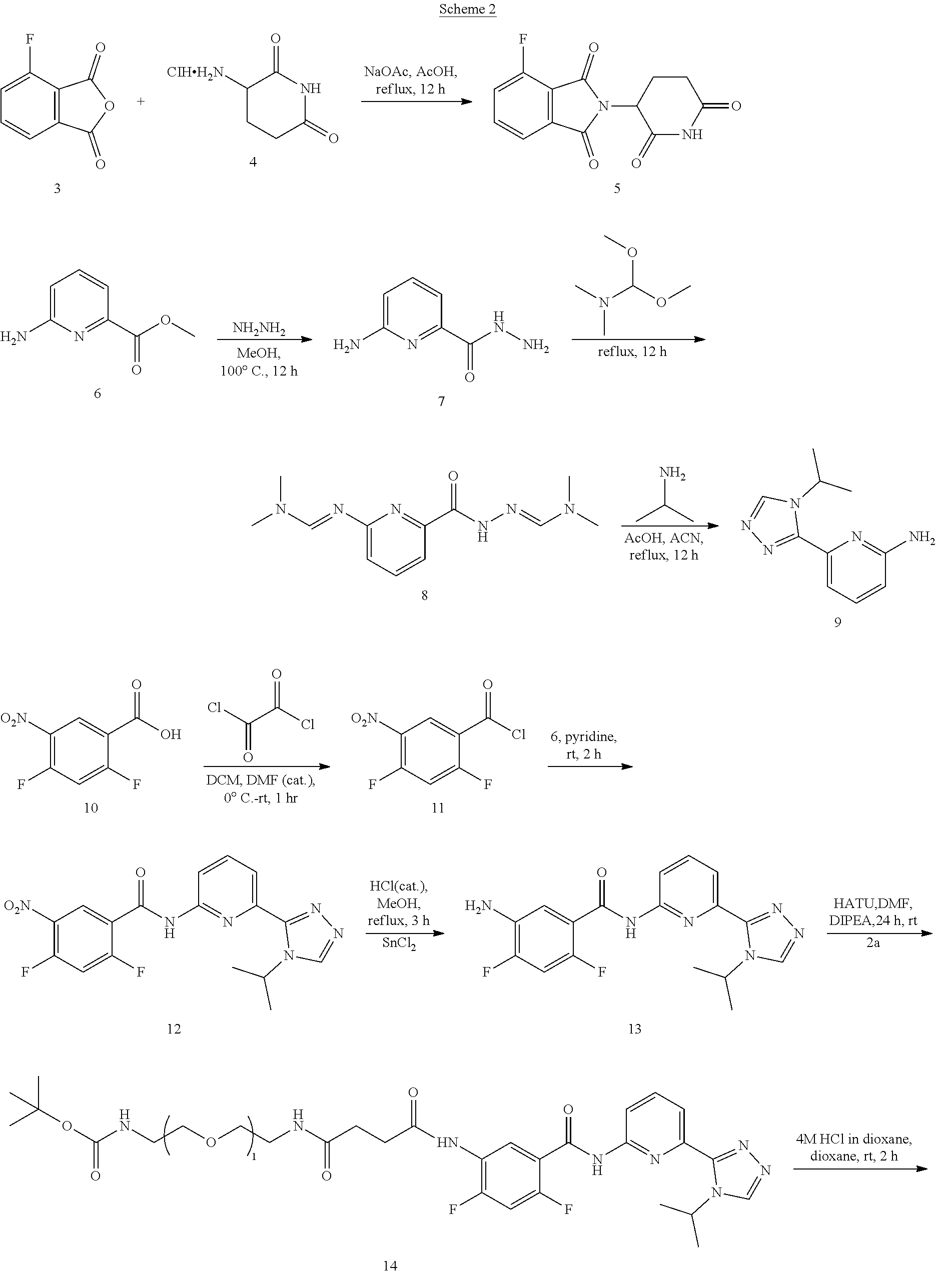

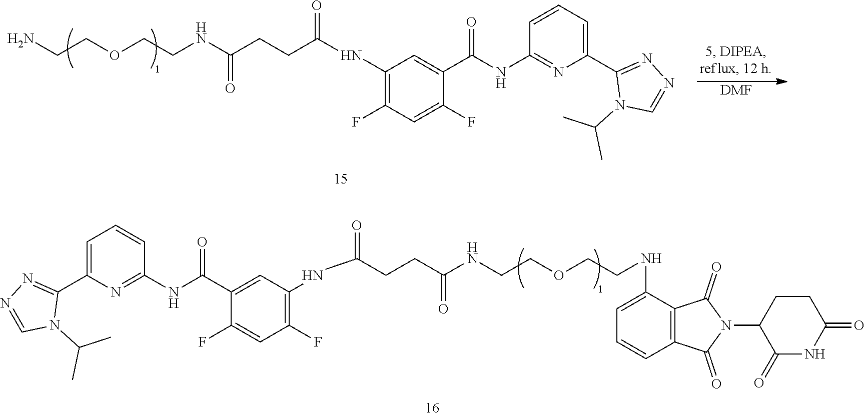

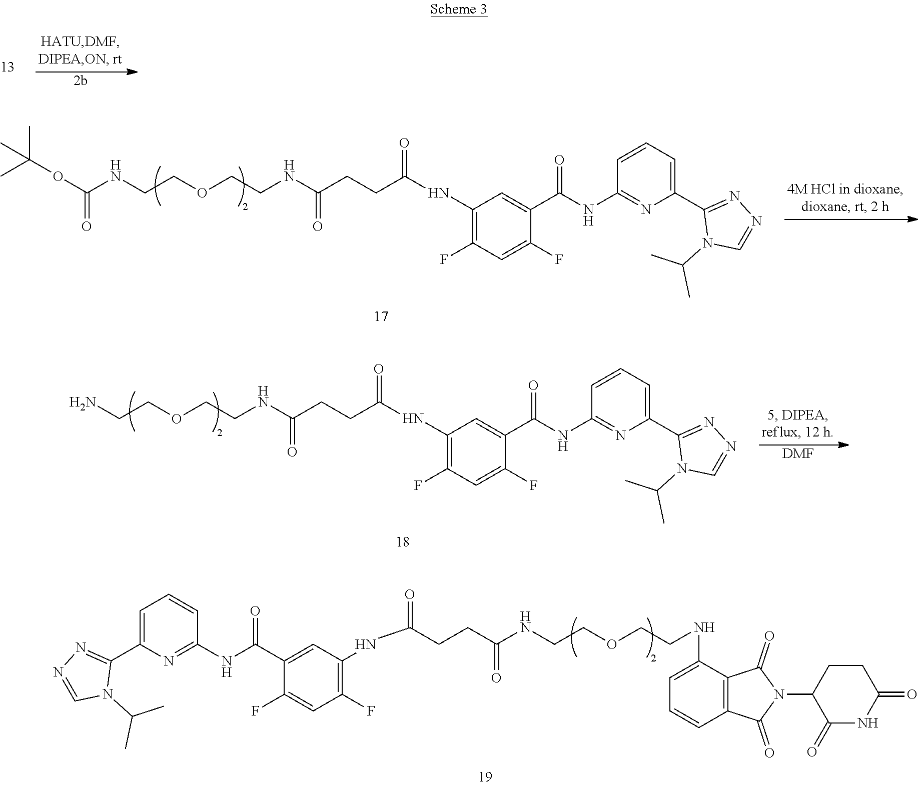

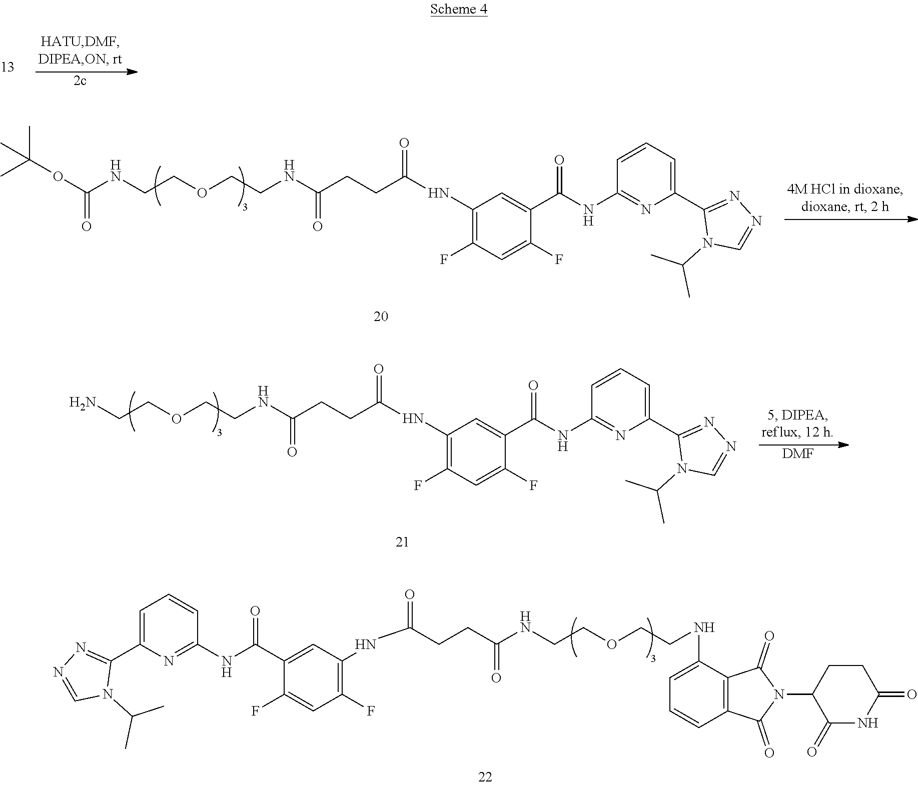

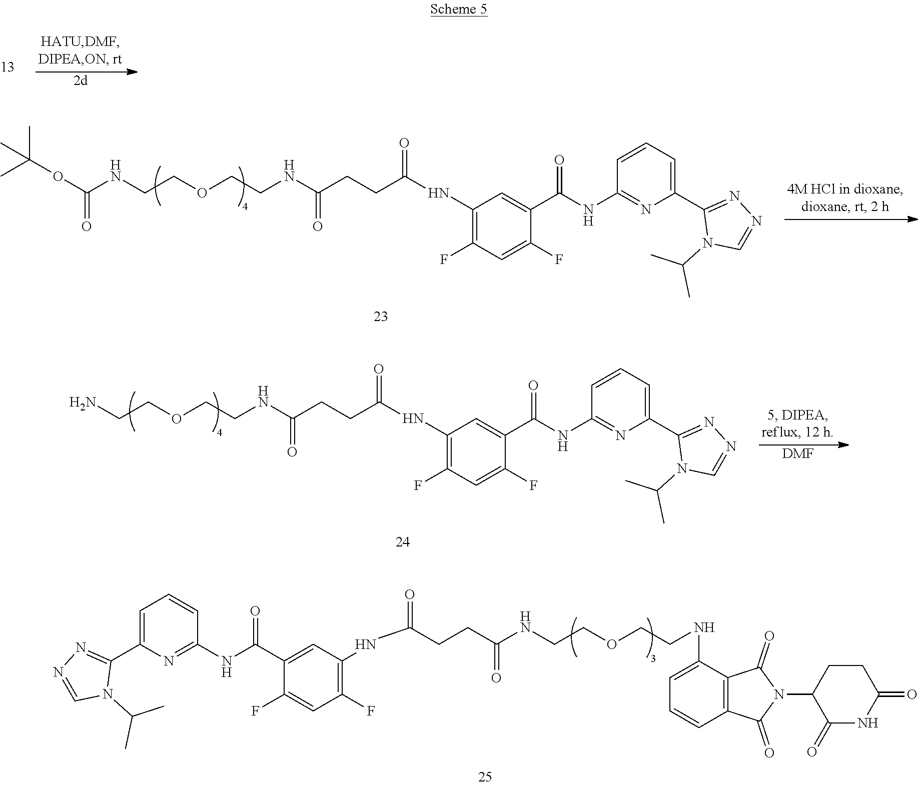

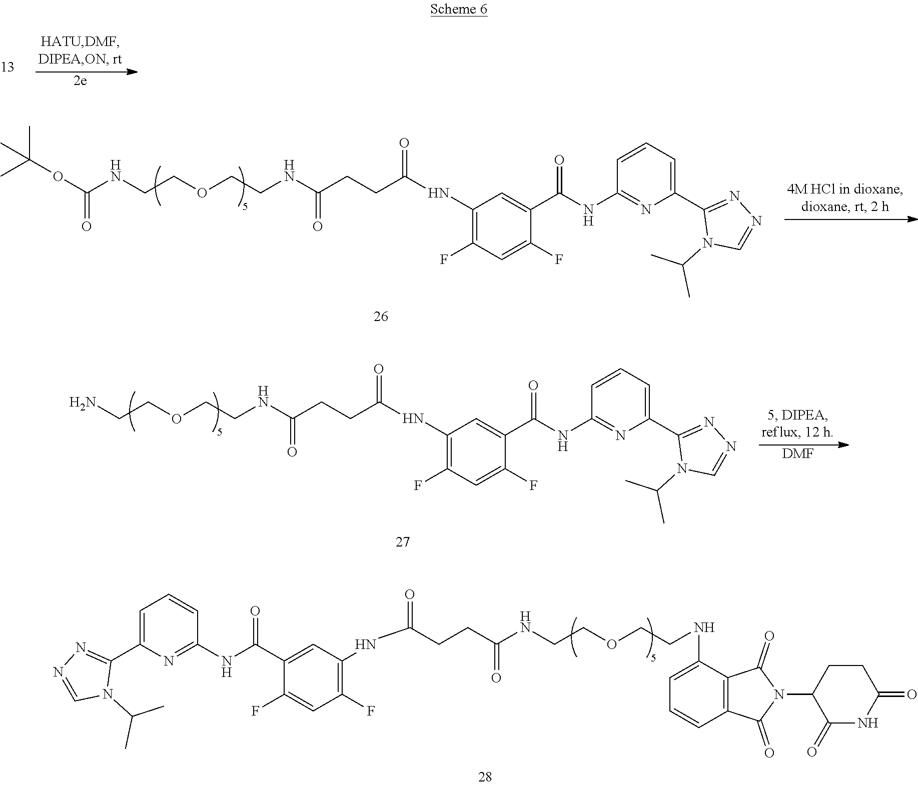

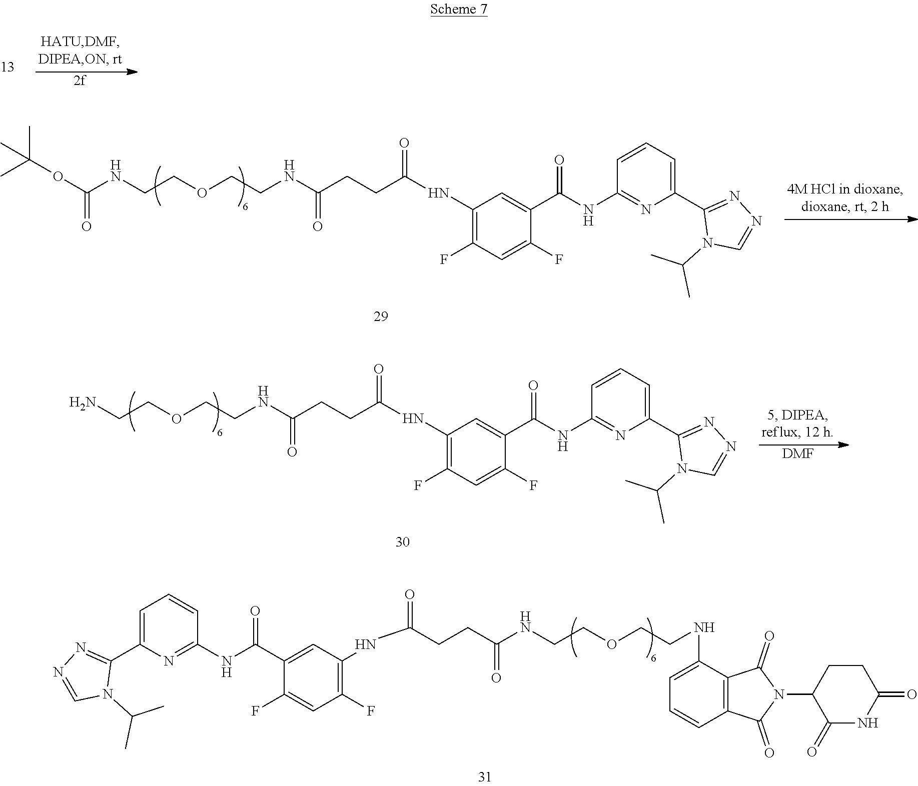

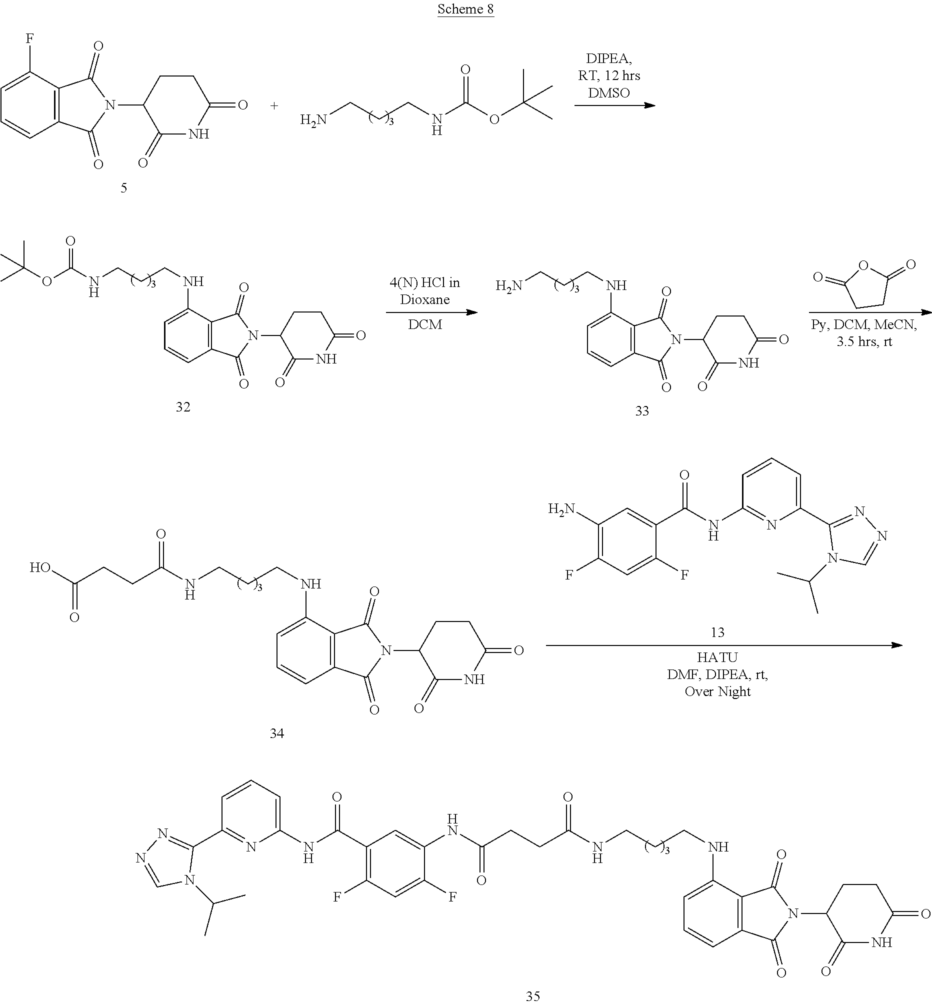

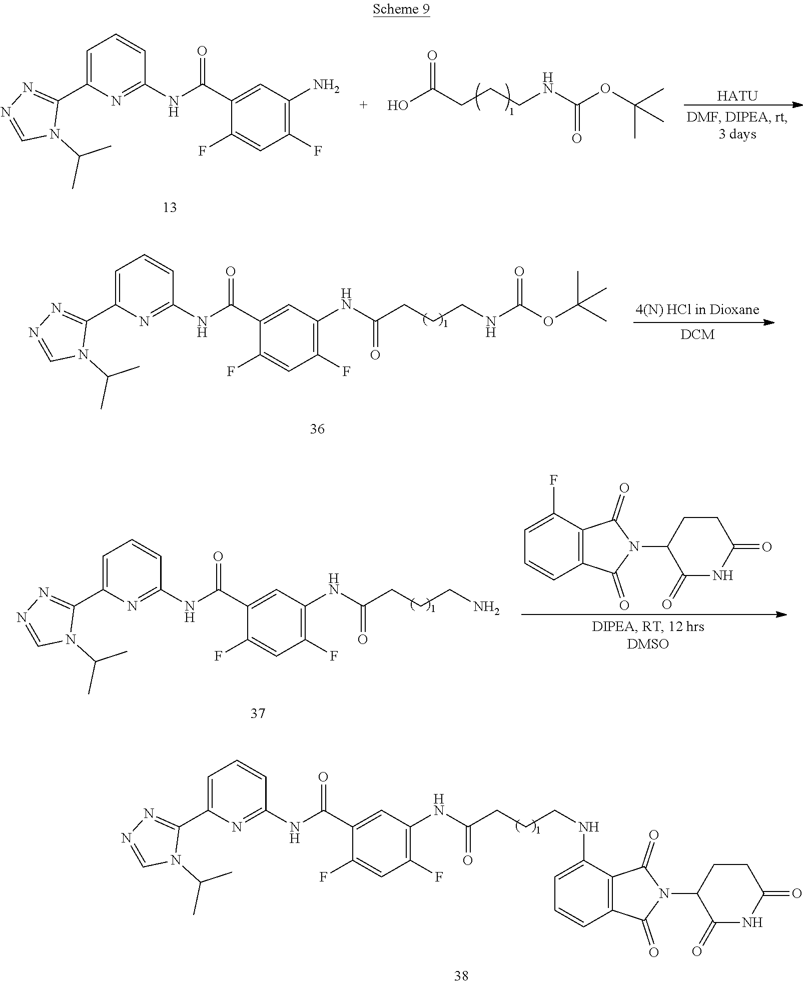

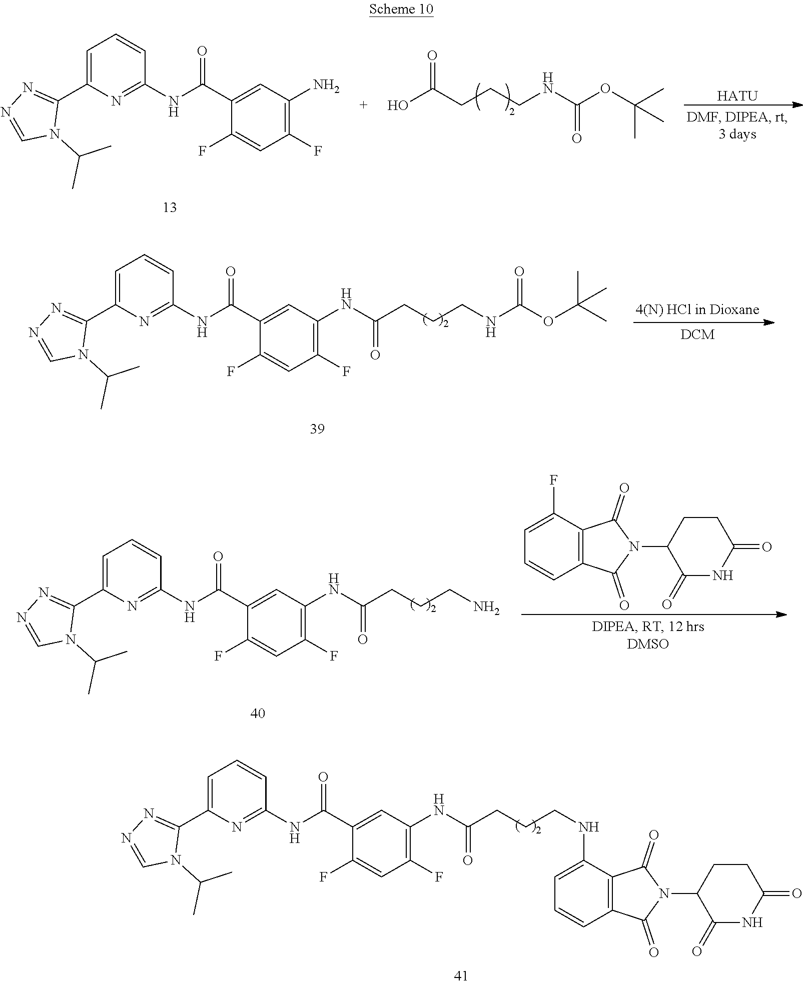

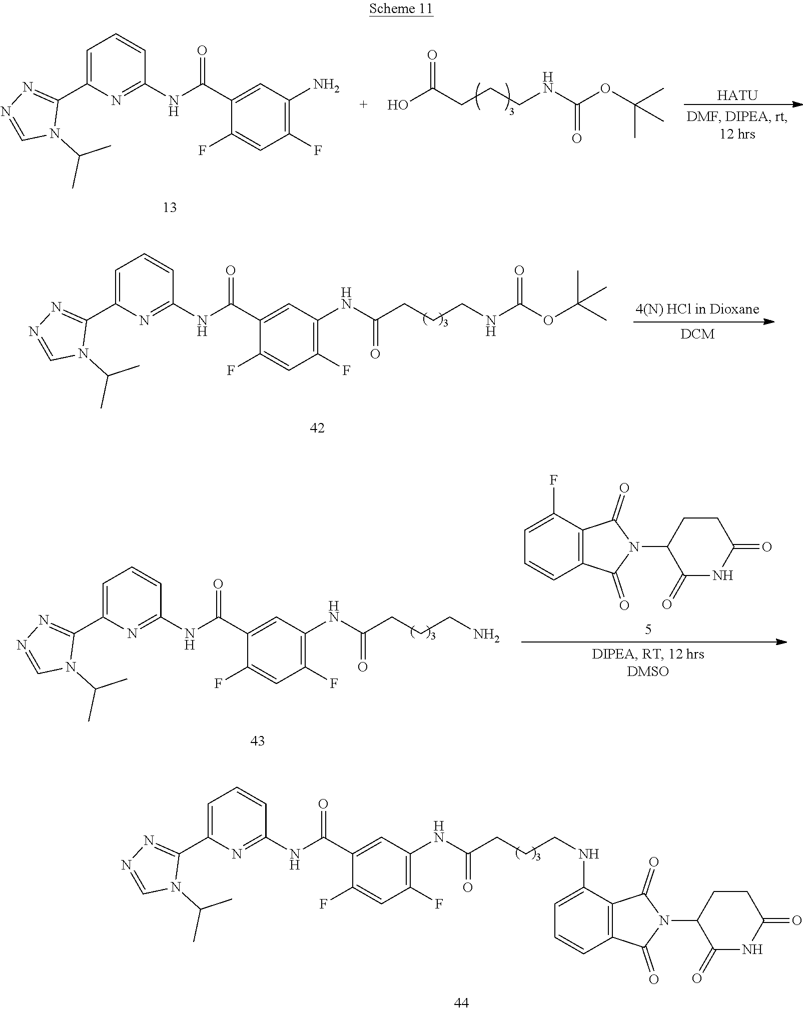

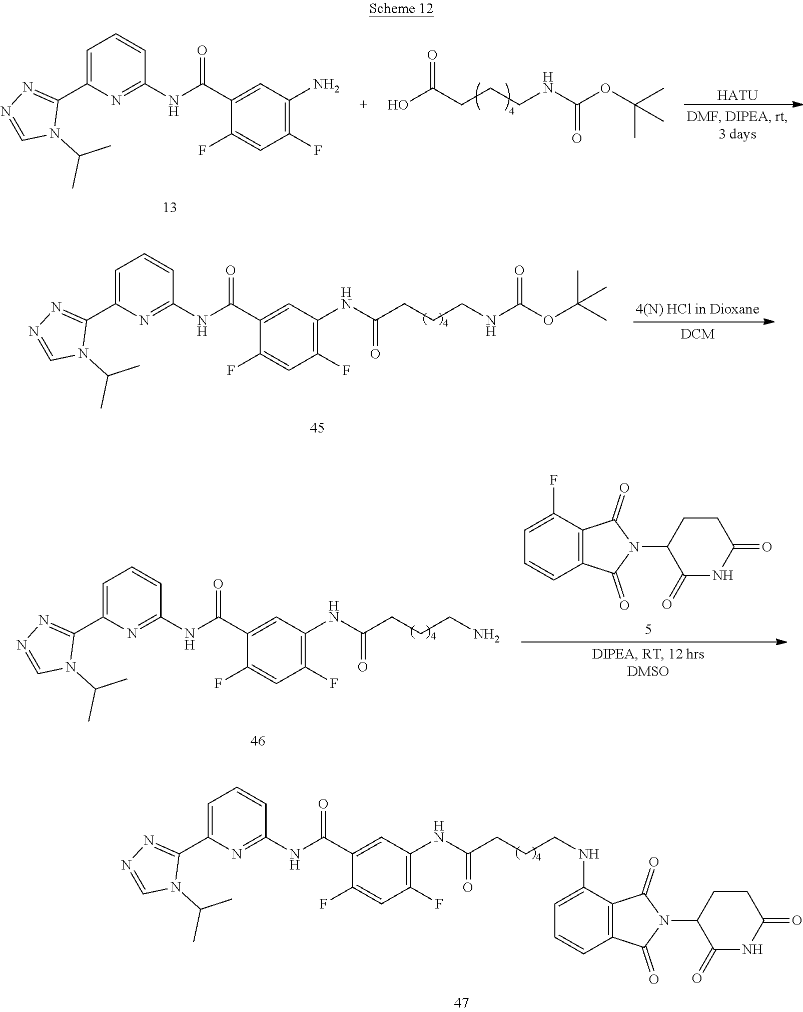

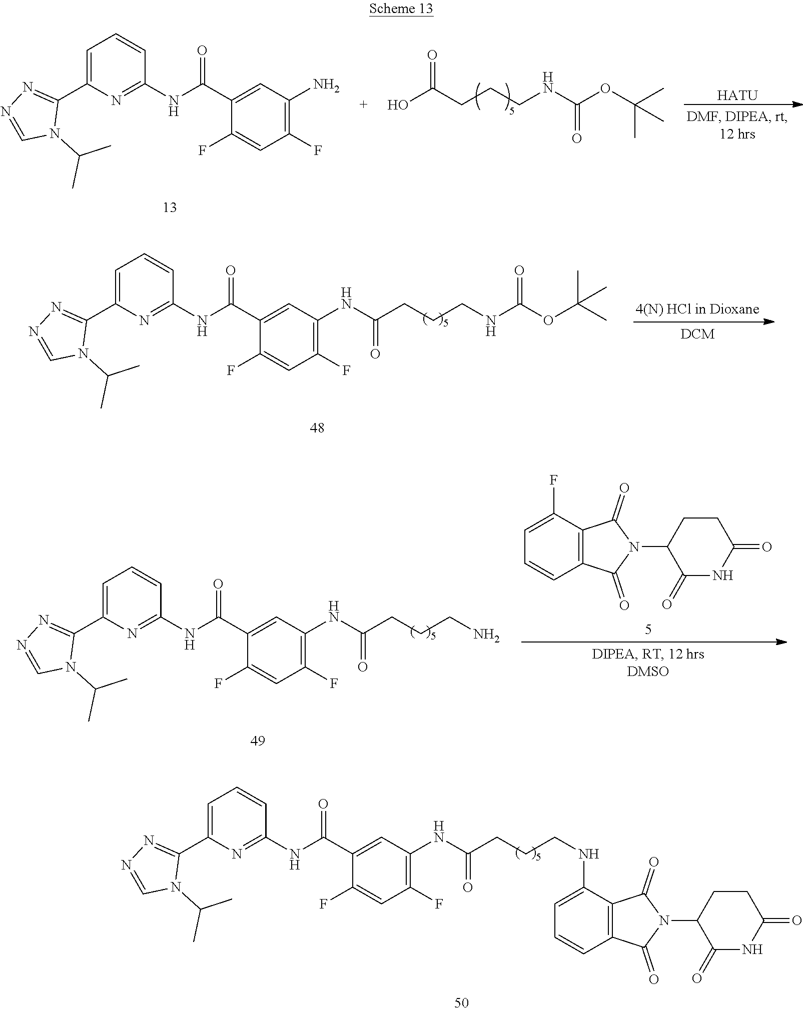

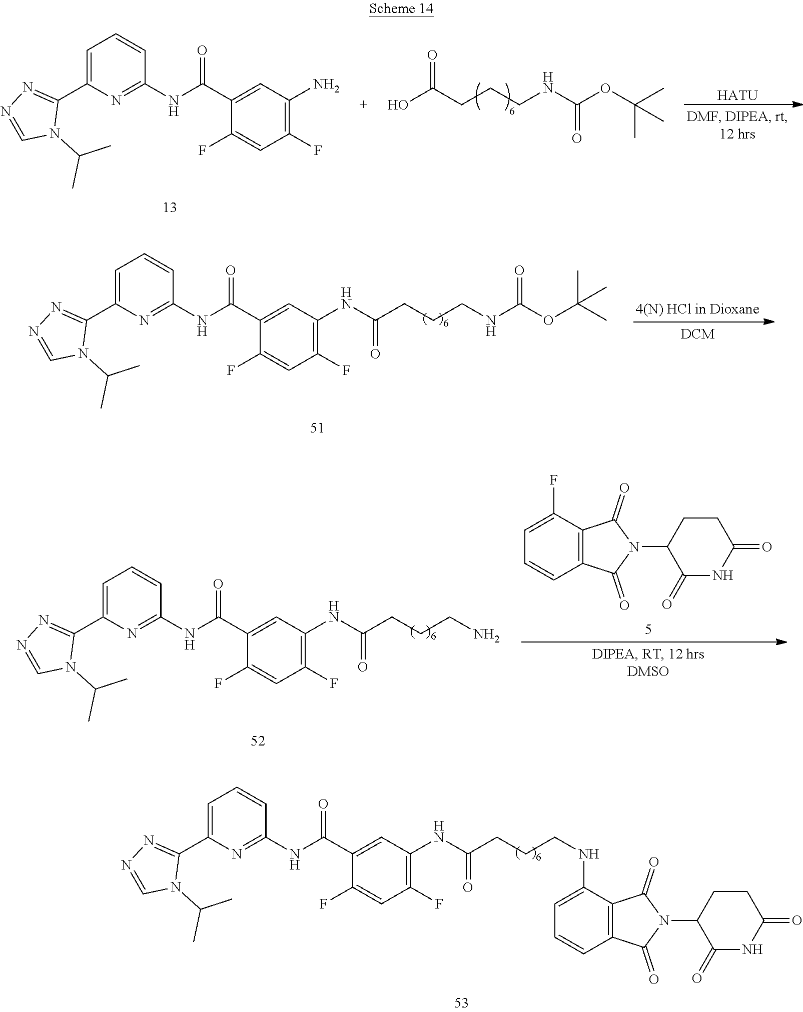

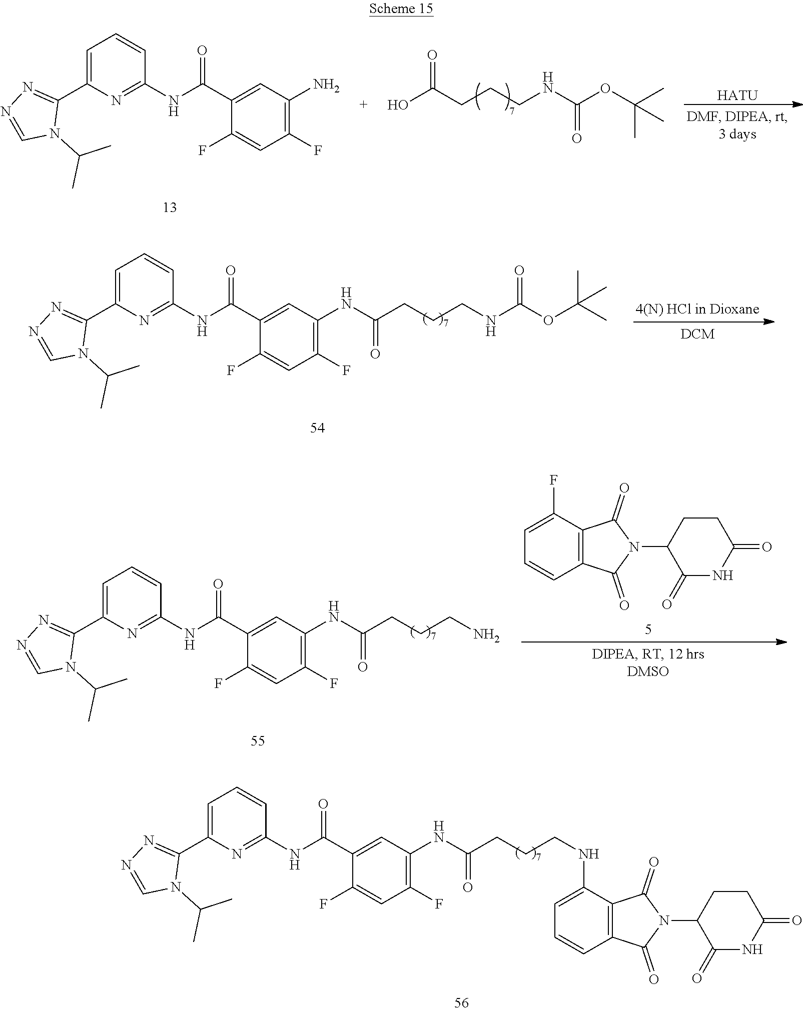

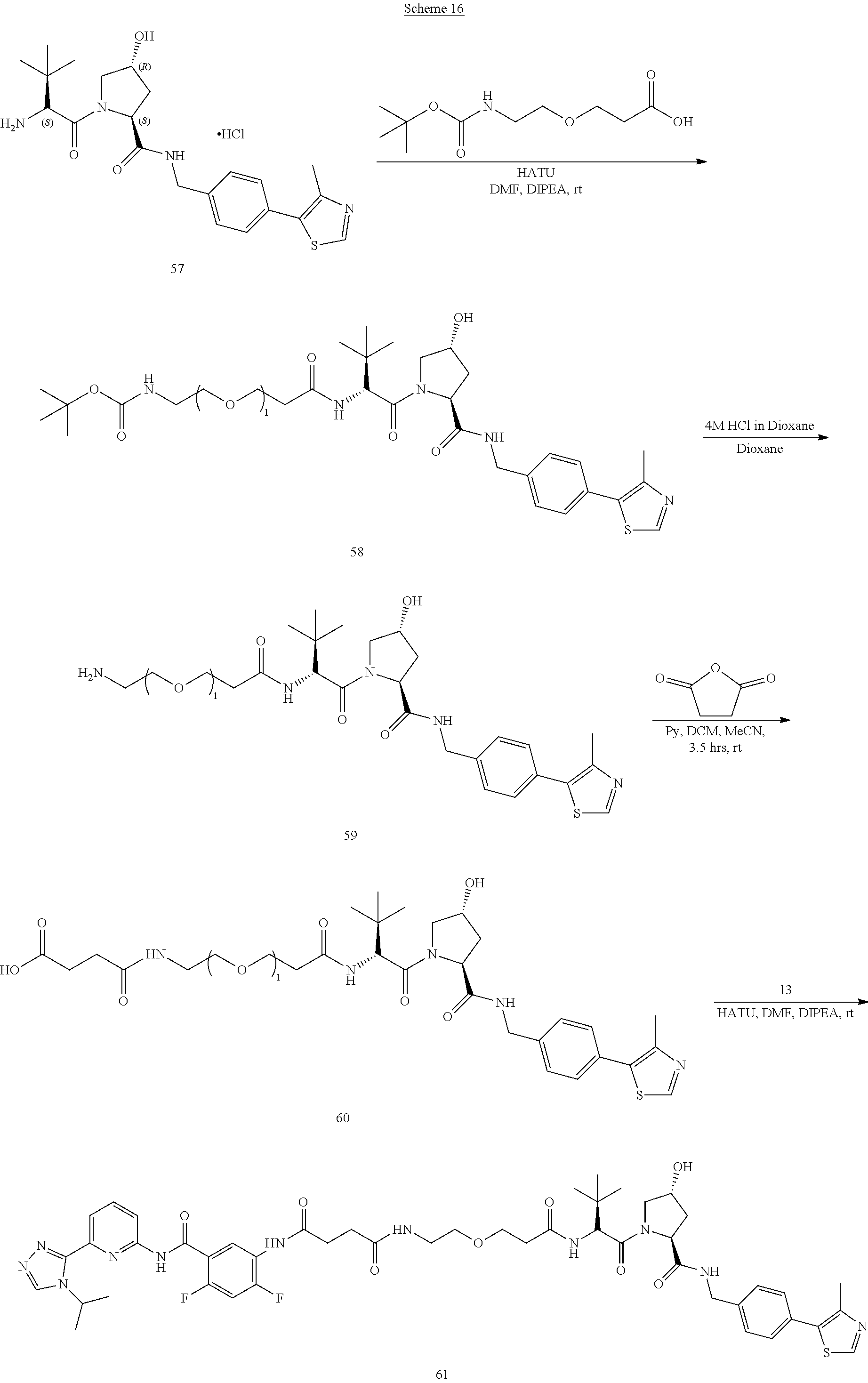

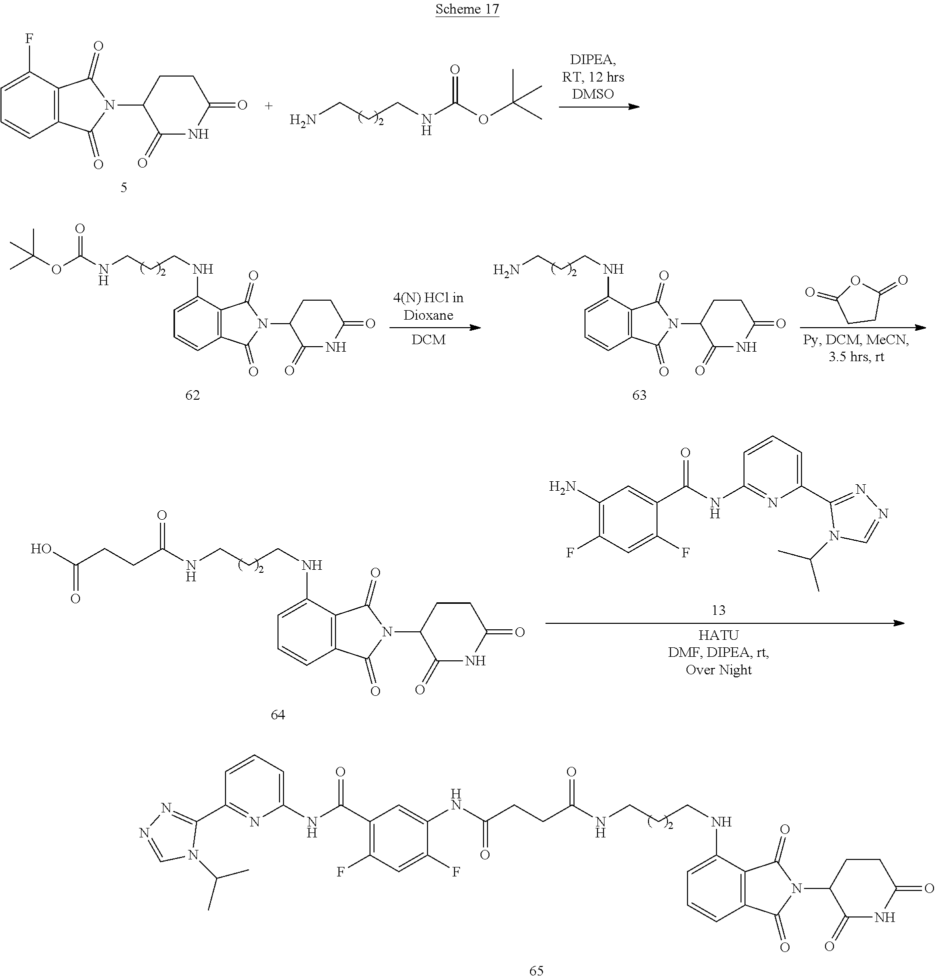

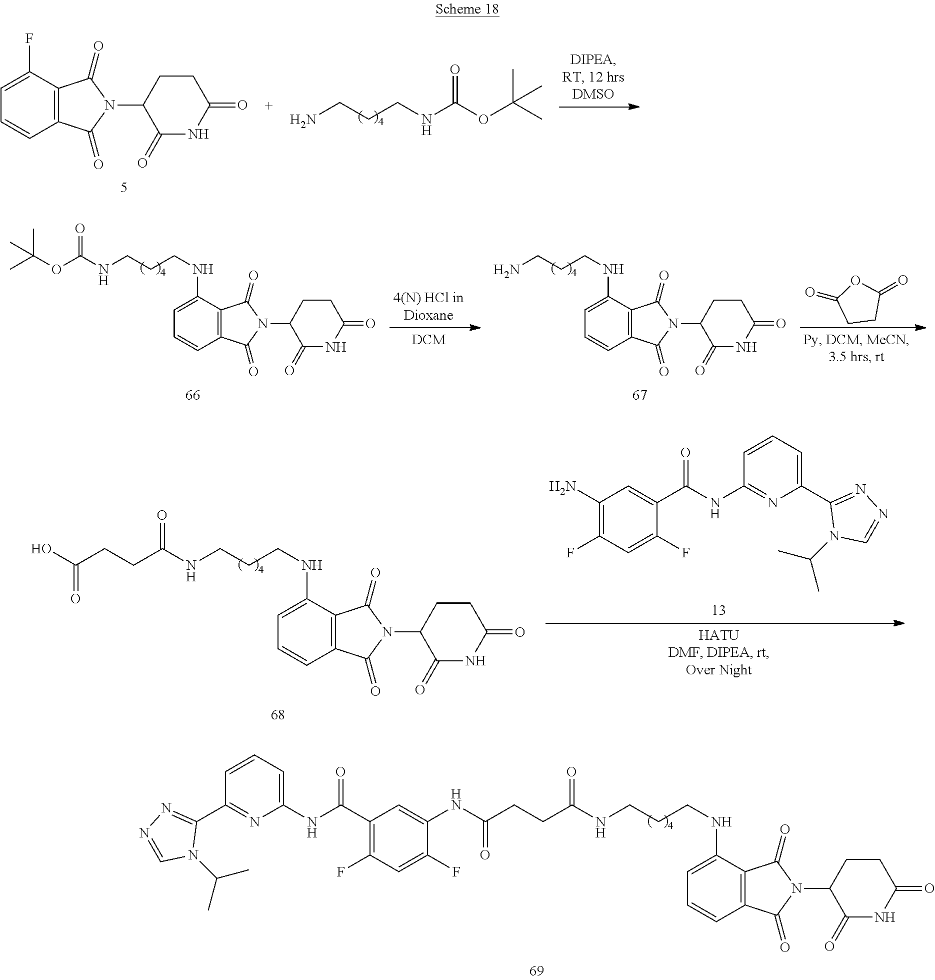

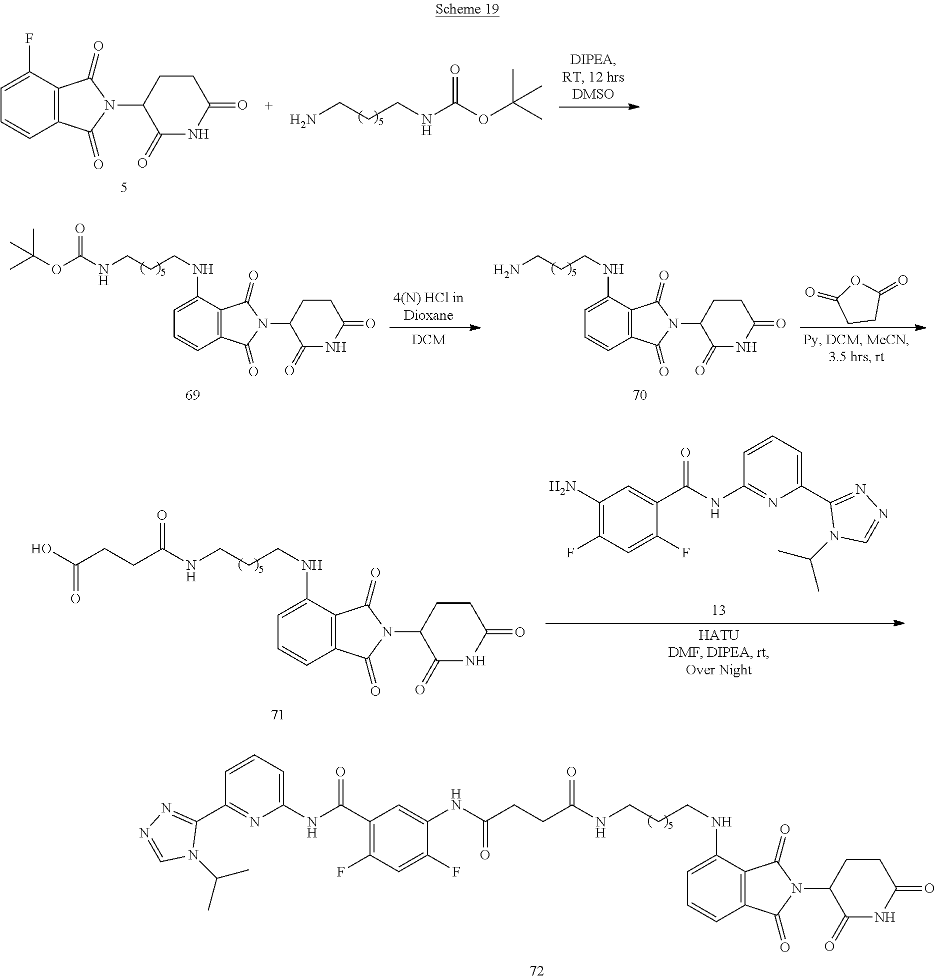

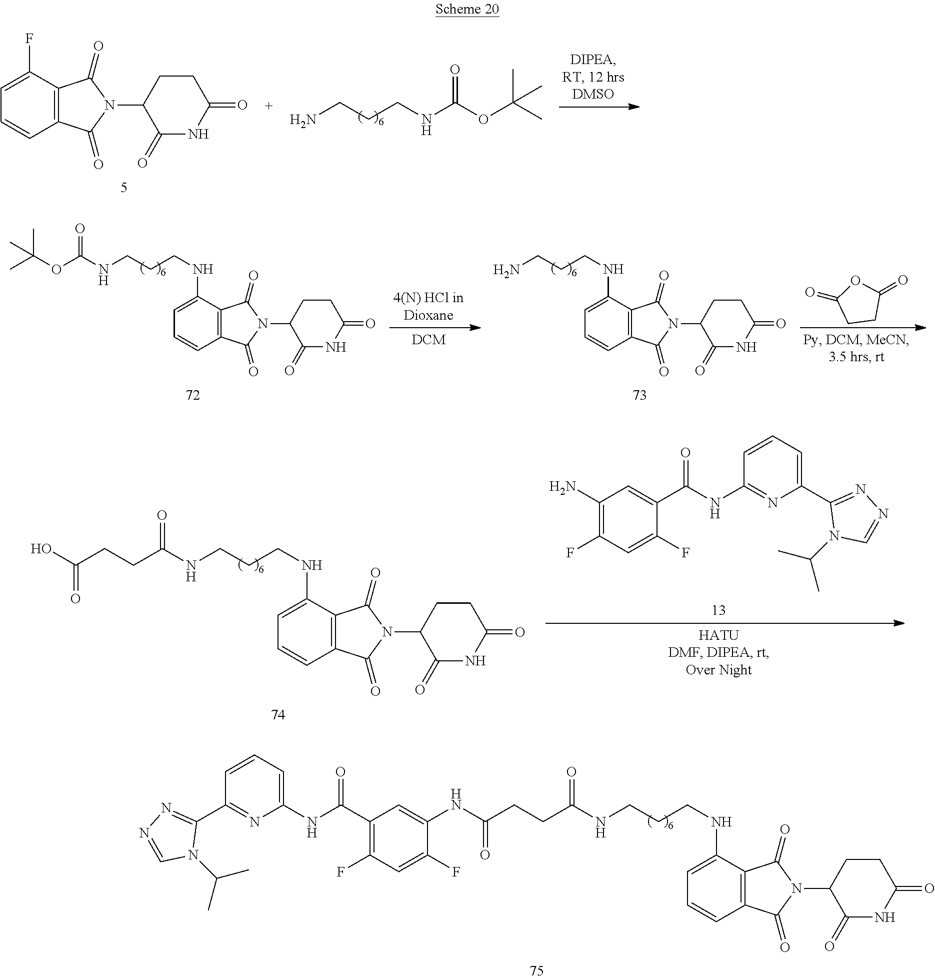

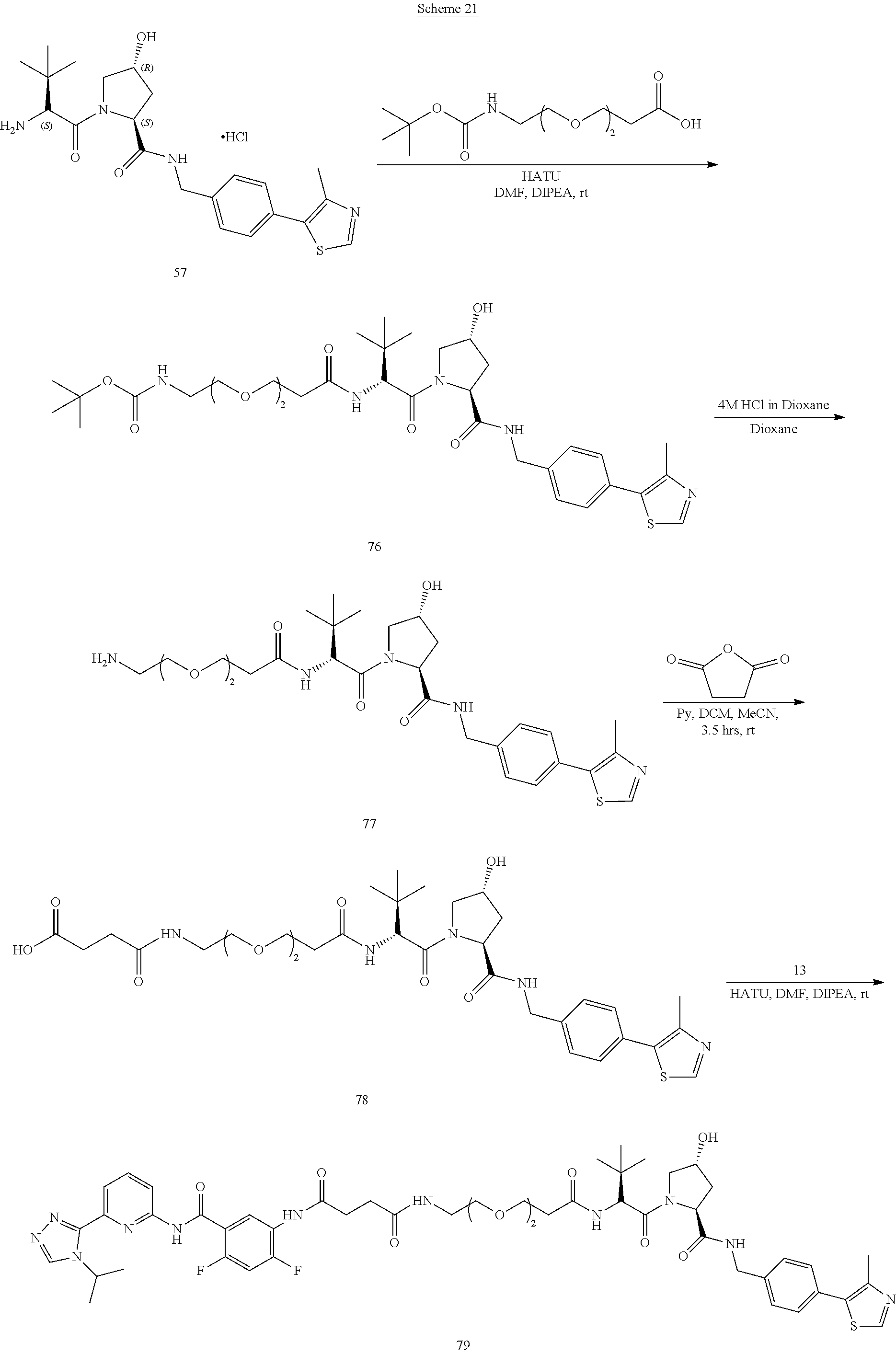

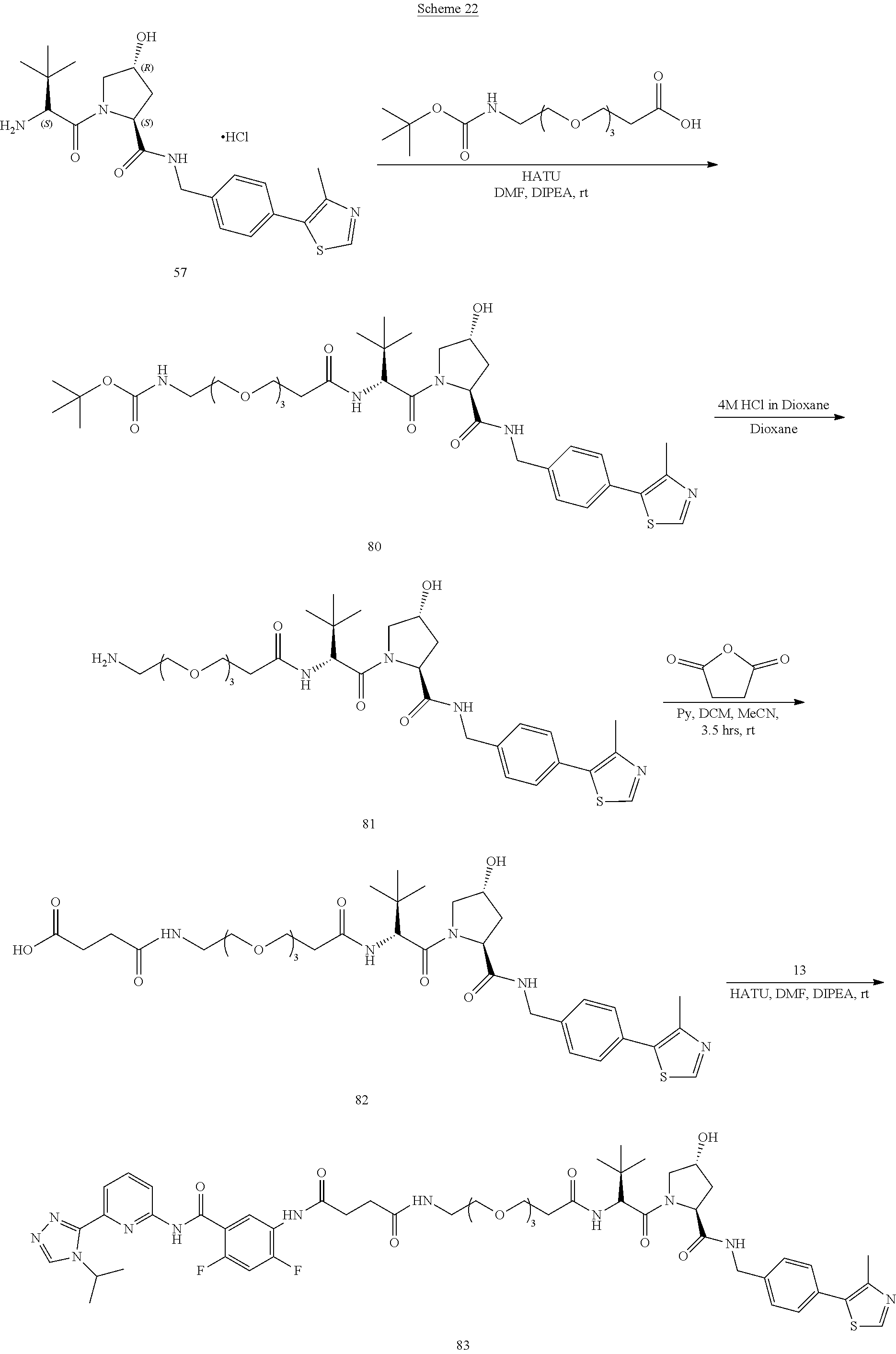

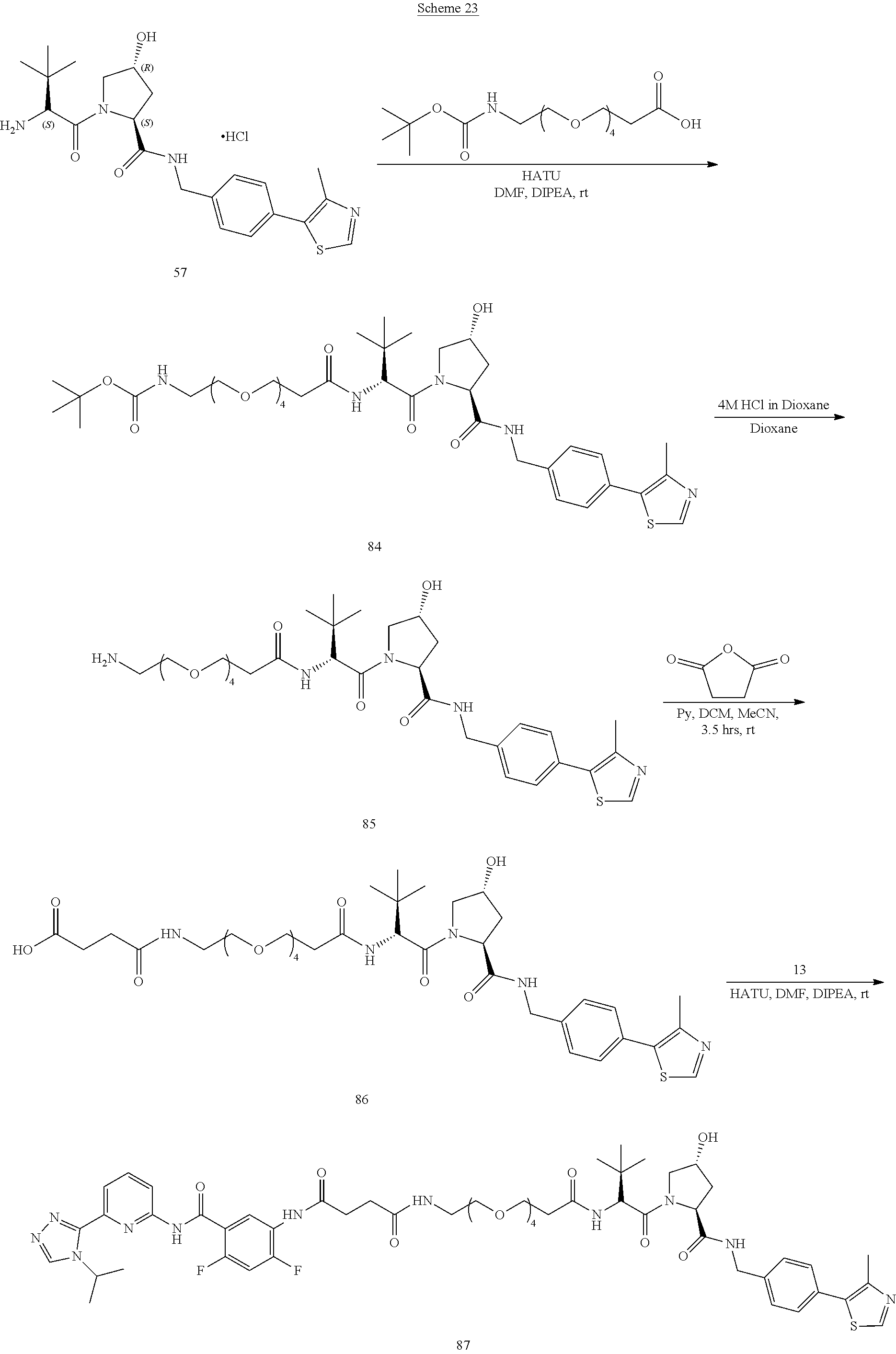

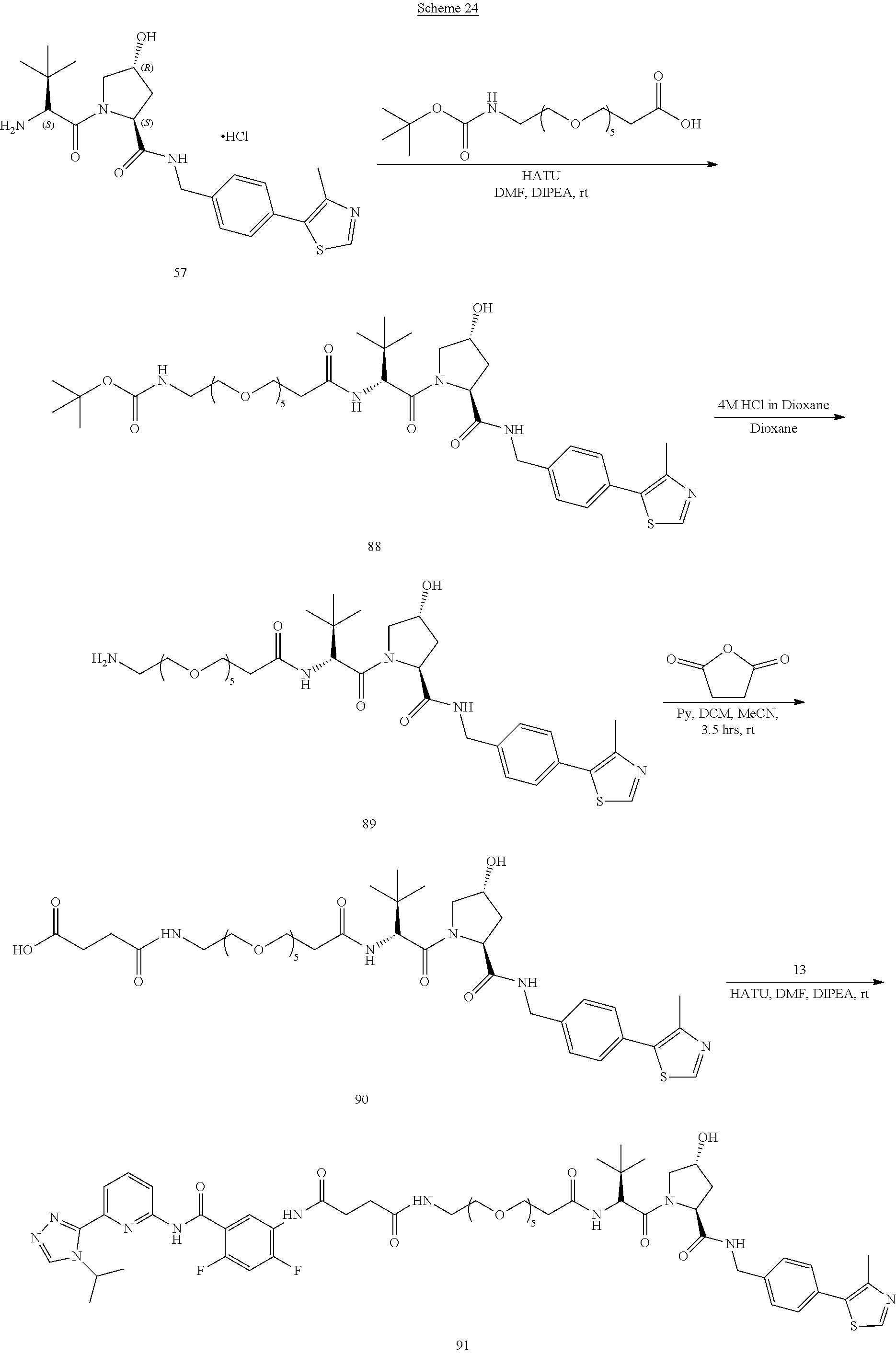

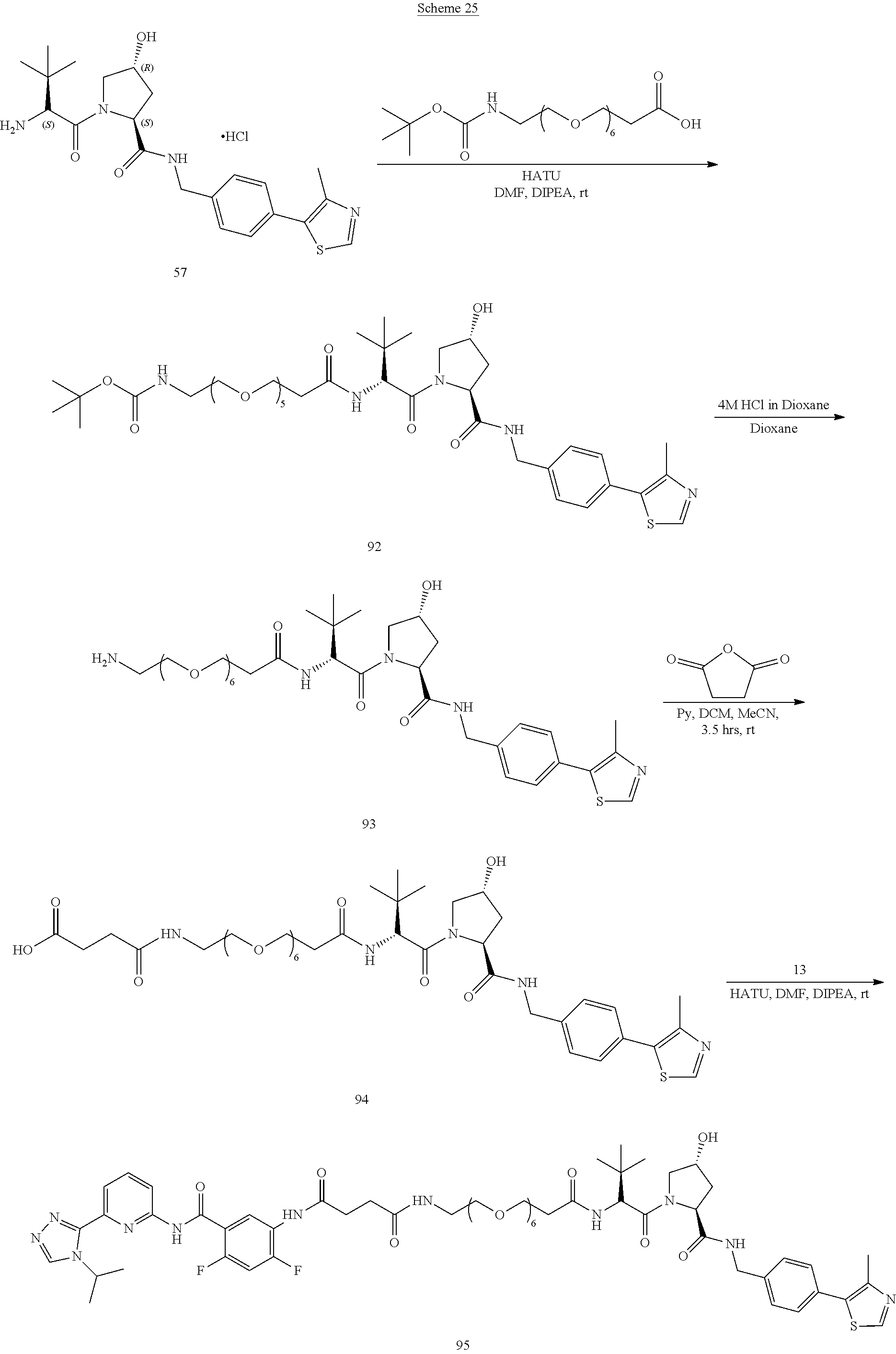

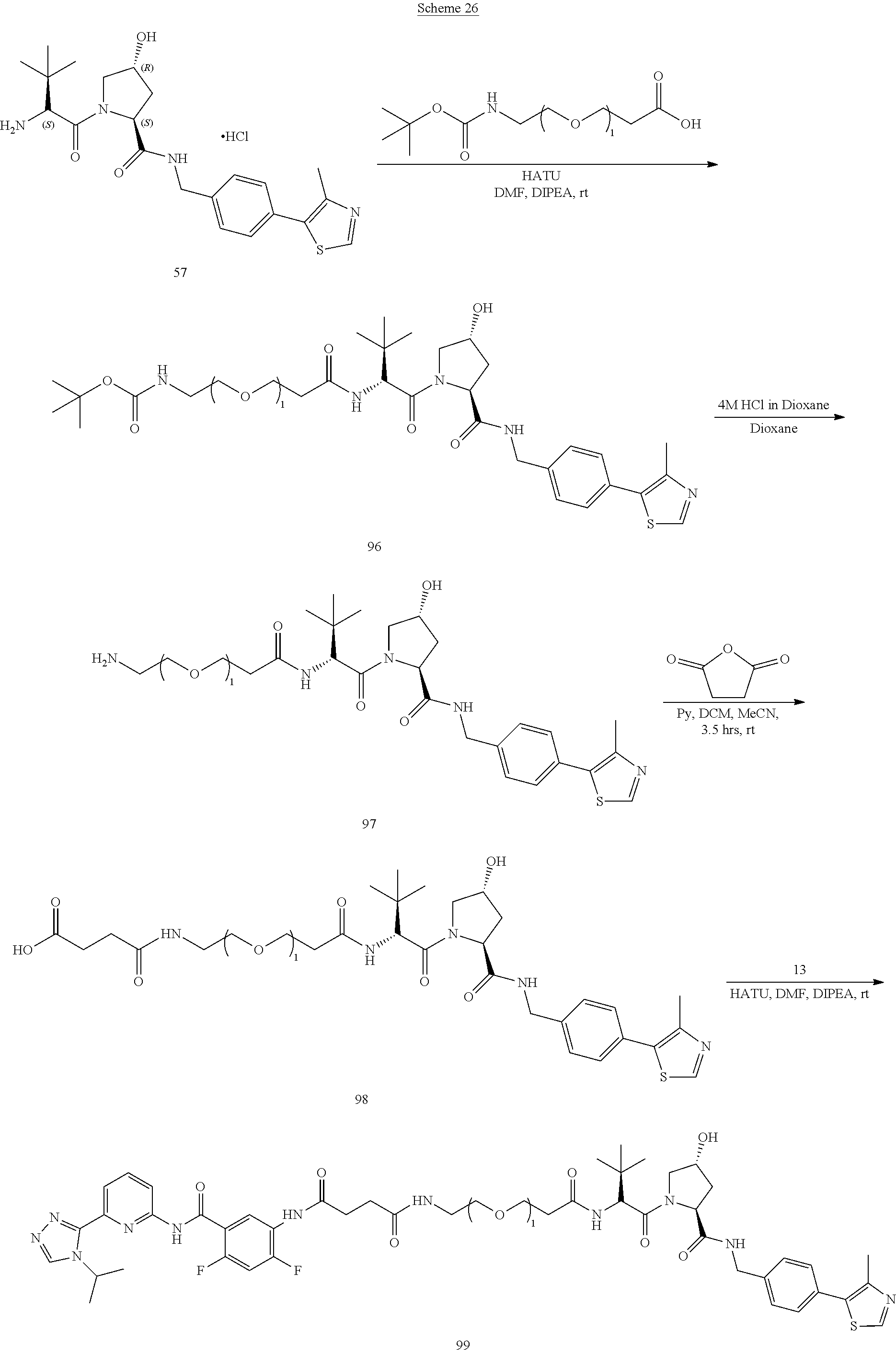

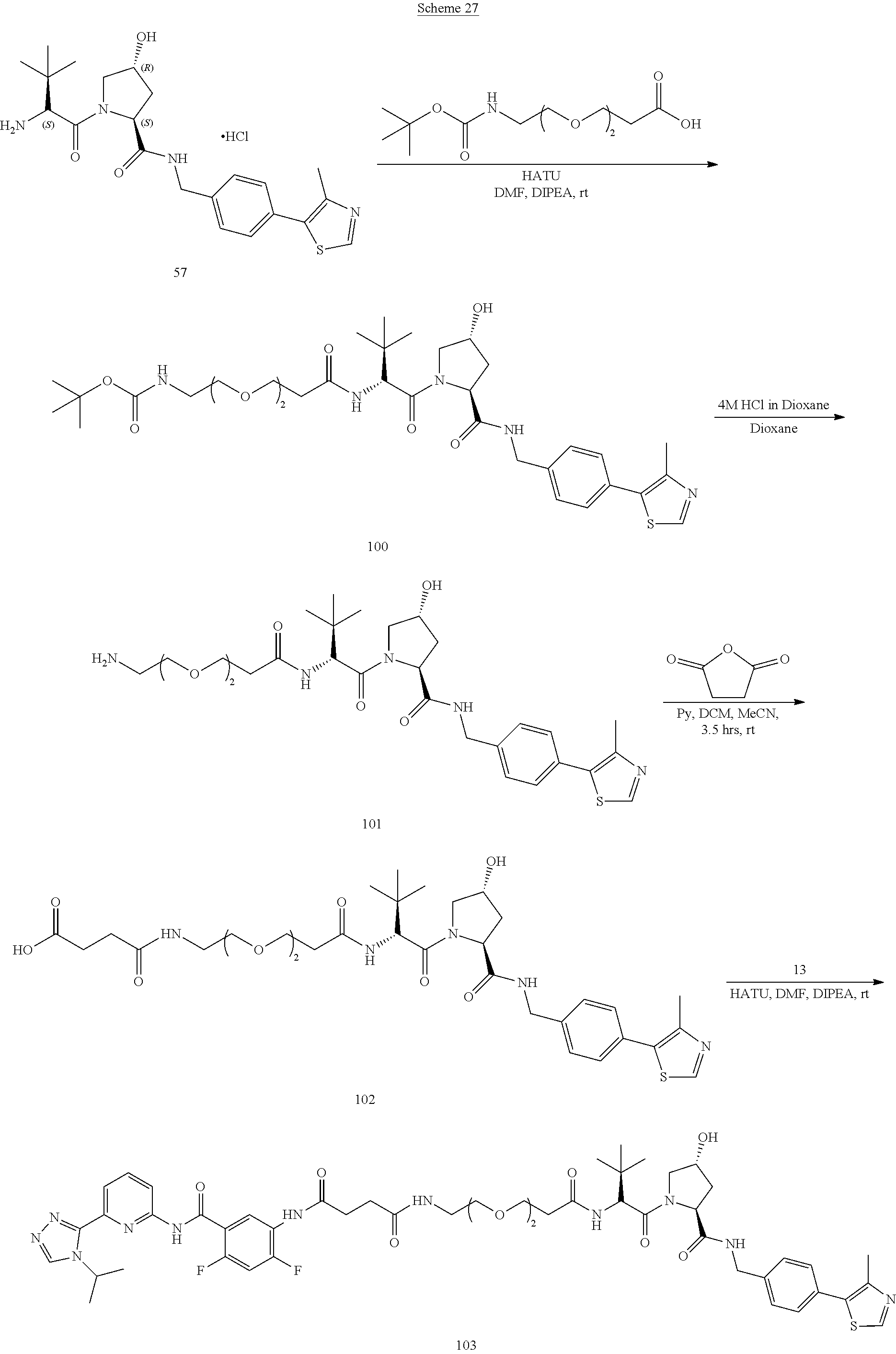

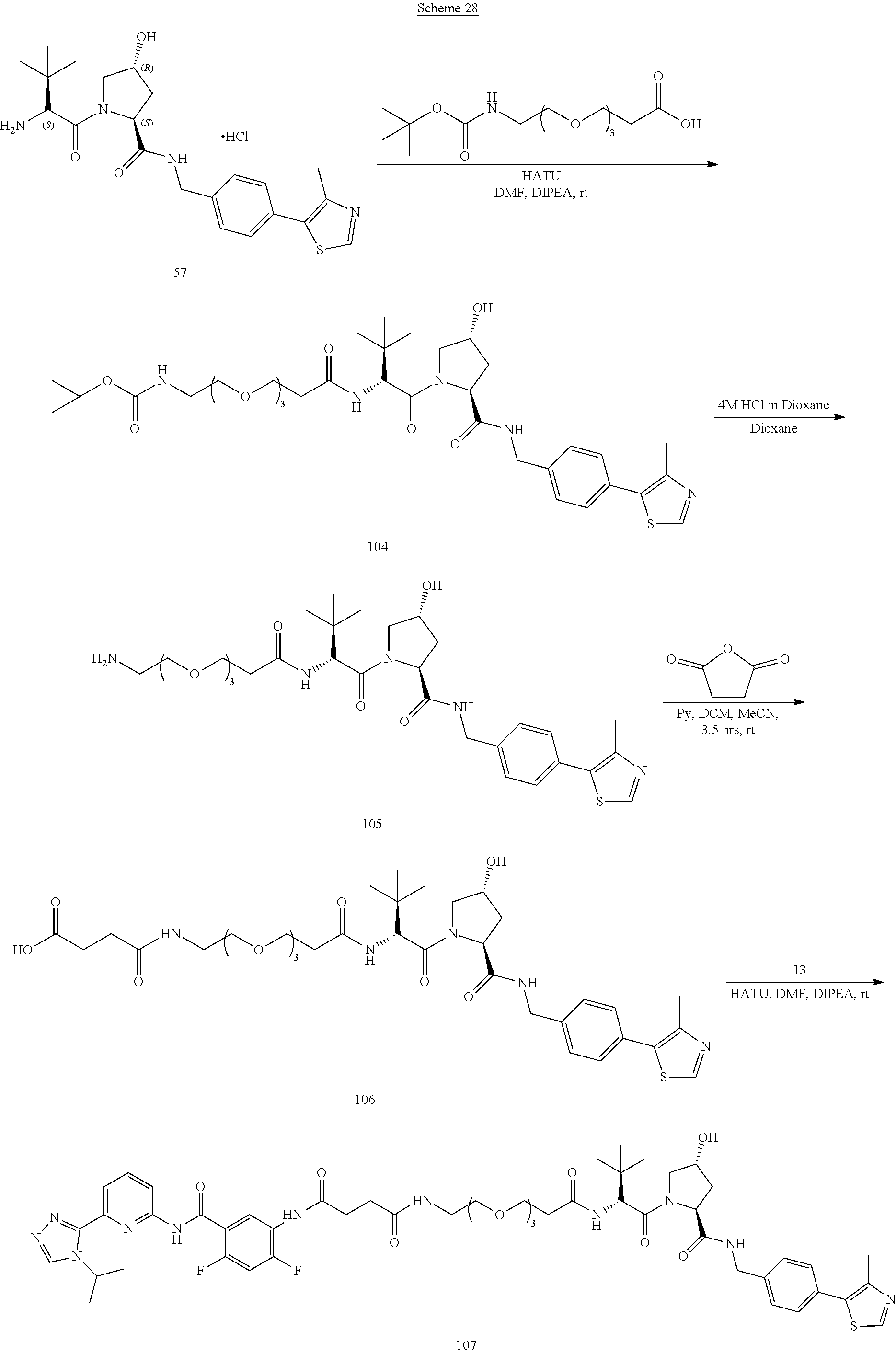

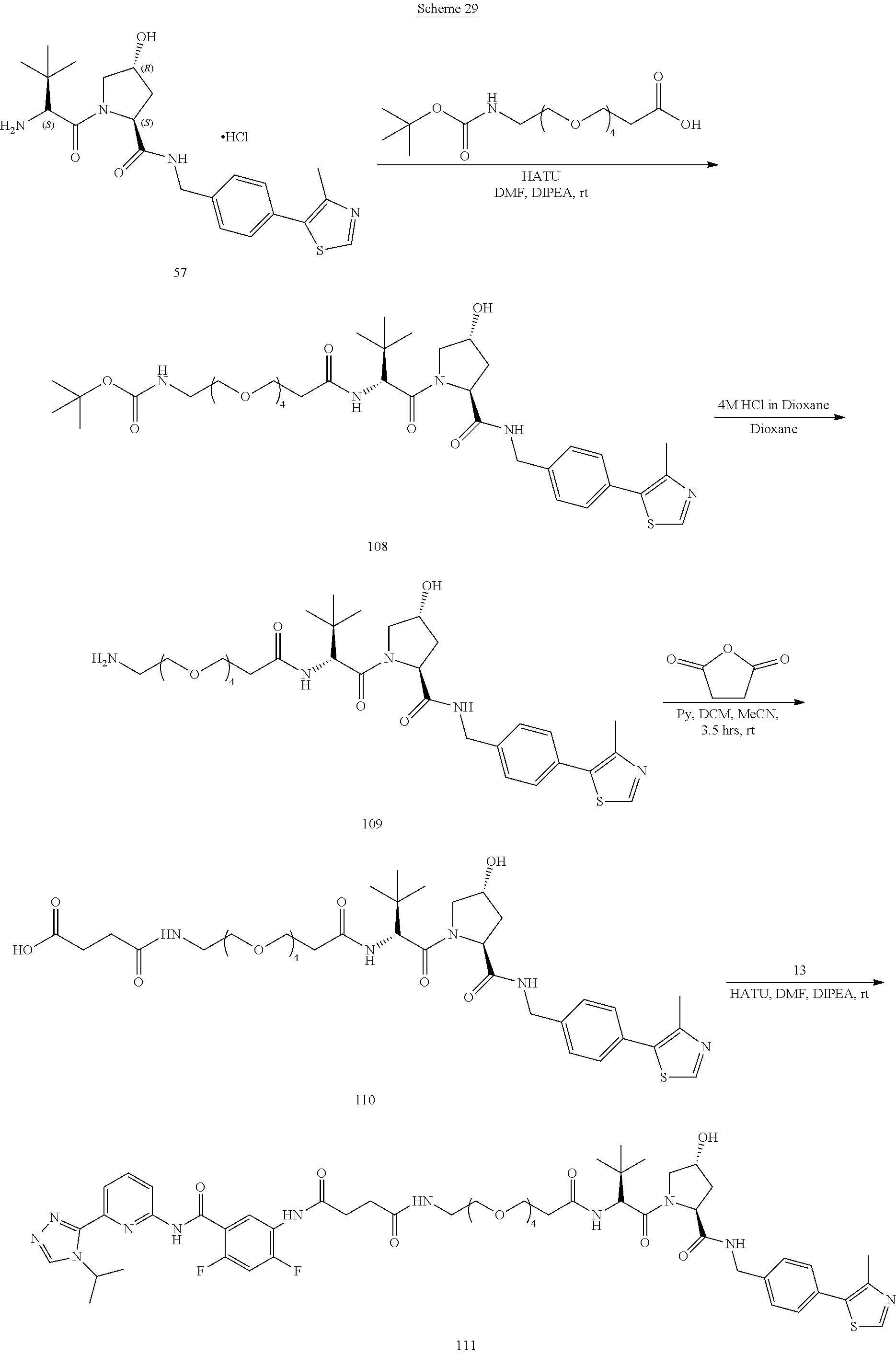

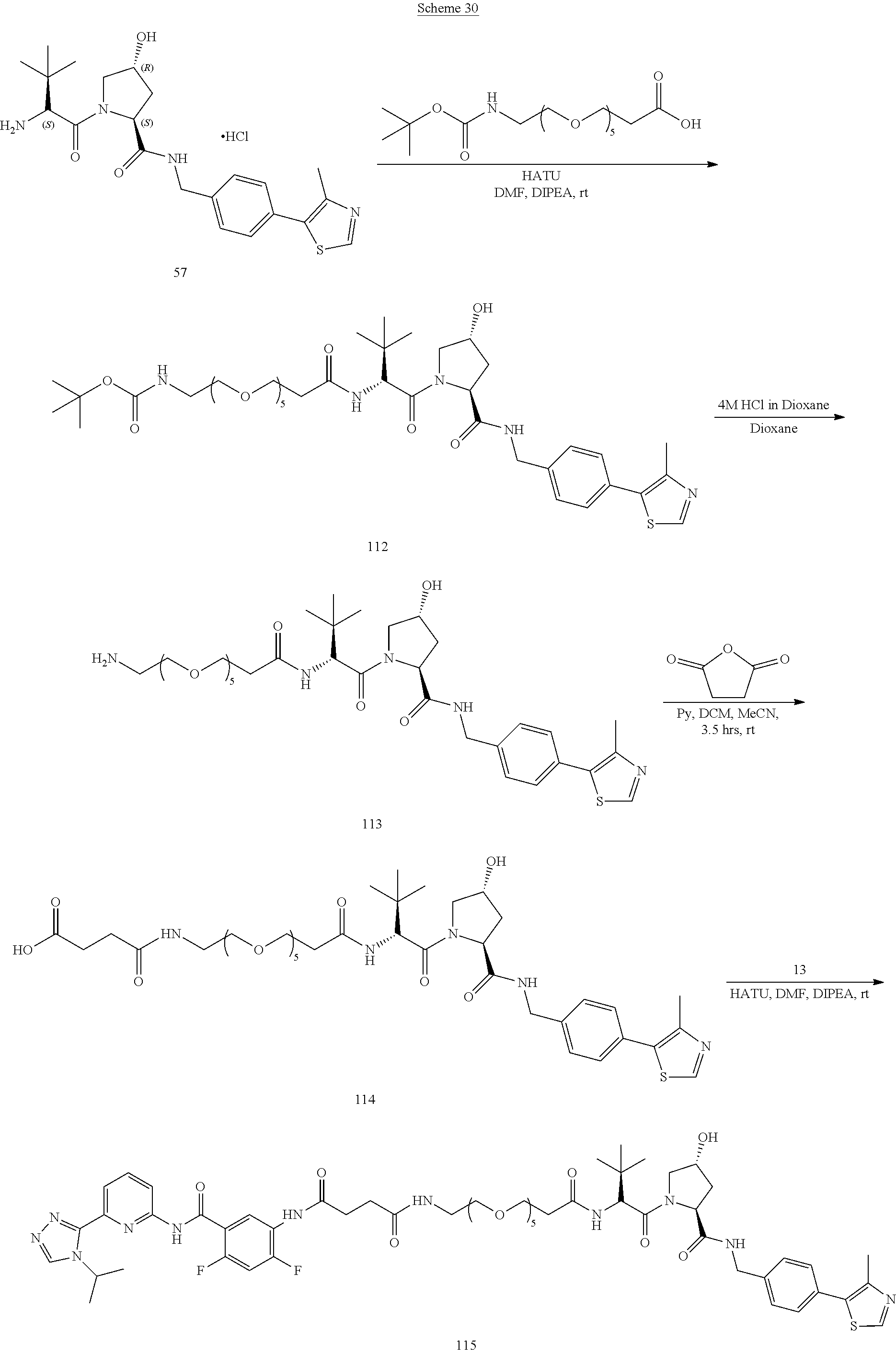

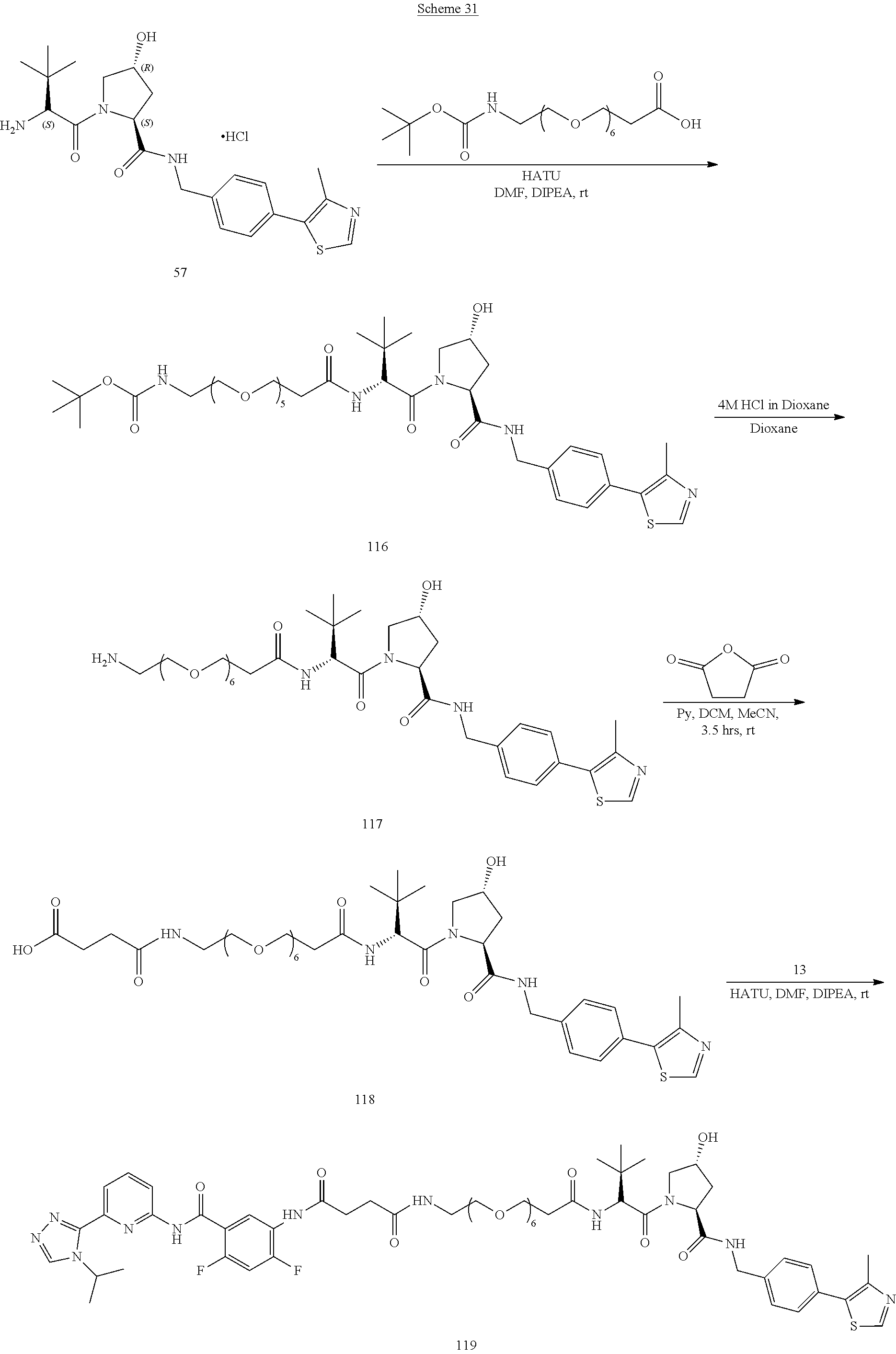

Another embodiment of the present invention is to provide a process for the design, synthesis, and optimization of a series of triazole-5-yl(pyridine-2-yl)benzamides compounds having structure I.

Still another embodiment of the present invention is to synthesize different compounds having the structure I with a spacer of different linker lengths.

Yet another embodiment of the present invention is to probe other E3 ligases ligands, such as CRBN and VHL to improve the scope of targeted protein degradation.

In yet another embodiment of the present invention, structure I are capable of hijacking E3 ligases and the ubiquitin-proteasome system (UPS) leading to selective degradation of the target protein apoptosis signal-regulating kinase 1 (ASK1).

In yet another embodiment of the present invention, compounds with structure I such as 16, 41 are heterobifunctional molecules made up of ASK1 target protein-binding ligand Selonsertib derivative and an E3 ligase (CRBN, VHL) recruiting ligand connected by a suitable intervening linker for ASK1 degradation.

Still another embodiment of the present invention is, wherein structure I such as 16, 41 are able to selectively degrade the target proteins ASK1 by changing different E3 ligase (CRBN, VHL) recruiting ligands.

Yet another embodiment of the present invention, is wherein structure I are able to selectively degrade the target proteins ASK1 by different type (such as PEG and alkyl) and different lengths of intervening linkers that connect target protein-binding ligand Selonsertib derivative and E3 ligase recruiting ligand(s).

Still another embodiment of the present invention is, wherein structure I ASK1 within 1-100 nM, leading to fast, selective, efficient, and prolonged degradation of ASK1 via cereblon (CRBN) E3-ligase in a proteasome-dependent manner in both HepG2 and HEK293 cells.

Yet another embodiment of the present invention is, wherein structure I provide a hypothesis for the structural basis of ASK1 degradation using cereblon (CRBN) by specific PROTAC through computational studies.

Still another embodiment of the present invention is, wherein structure I are capable of targeted degradation of ASK1 have potential in patients with hepatic fibrosis and in nonalcoholic fatty liver disease (NAFLD)/nonalcoholic steatohepatitis (NASH) research where modulation of therapeutic target ASK1 is important.

Still another embodiment of the present invention is to visualize the selective protein degradation by western blotting, fluorescence microscopy imaging, and proteomics.

Yet another embodiment of the present invention is to analyse and validate the structural basis of ASK1 degradation using CRBN by specific PROTAC through computational studies.

Still another embodiment of the present invention is examining the ternary complex formation in cells by fluorescence microscopy imaging.

Yet another embodiment of the present invention is to apply the active PROTAC compounds in both proteasomal stress-induced in vitro assay and preclinical NASH models.

Still another embodiment of the present invention is to provide a composition comprising compounds of structure I for use in several clinical applications, including pharmaceutical agents and methods for treating disease conditions like NAFLD/NASH.

### Computational Analysis

To understand the binding pattern of the PROTAC-16 and to explore its sustainability in keeping the proteins CRBN and ASK1 in close proximity, computational approaches were exploited using protein structures of E3-ligase ternary complex [PDB: 5FQD; containing Cereblon (CRBN), DNA Damage Binding Protein 1 (DDB1), Caesin Kinase 1 (CK1)] and ASK1 [PDB: 6OYT; containing ASK1 Kinase Domain].

The thalidomide moiety of the compound 16 was docked into CRBN of PDB: 5FQD and the docked pose was found to be similar to that of the cocrystallized ligand Lenalidomide, involving the H-bond interactions with His 378, Trp 380, Asn 351, Ser 379 and Trp 386. Similarly, the selonsertib-derived moiety was separately docked into the protein structure of ASK1 (PDB: 6OYT), where H-bond interactions were observed with residues Lys 709, Val 757, Gln 756 and Asp 822. The solvent-exposed part of the selonsertib moiety was strategically modified for the incorporation of the linker chain as the modification of other positions of selonsertib may alter the ASK1 receptor recognition. Thus, the solvent-exposed cyclopropyl-imidazole substructure was removed in the PROTAC design to connect with the E3 ligase ligand thalidomide via a suitable linker. Next, anticipating the functional and geometric similarity of the kinase domain, the casein-kinase 1 of the ternary complex of PDB: 5FQD was replaced by the ASK1 kinase domain (containing the docked Selonsertib-derived moiety). It revealed that the two docked ligands thalidomide moiety onto CRBN and Selonsertib-derived moiety onto ASK1) point towards each other, through their corresponding terminal primary amines at solvent-exposed interphases of each protein, indicating that it might be the convenient position for linker growth. The complex was optimized with varied distance constraints followed by manual modelling of the linker, following the protein-surface topology. To optimize the structure of the linker and the manually modelled complex (DDB1, CRBN-docked ligand, and ASK1-docked ligand), it was further energy minimized and the binding free-energy was estimated by MMGBSA (Molecular mechanics with generalised Born and surface area solvation). The PROTAC conformation with an intermediate distance of 12 Å between ASK1 and CRB showed the least MMGBSA score, thus considered the most stable conformation. The PROTAC compound 16 retained the H-bond interactions with Trp380, His 378, Asn 351, and Trp 386 with CRBN through its thalidomide moiety and with Lys 709, Gln 756 and Asp 822 of ASK1 through selonsertib-derived moiety of compound 16. Additionally, the ethereal oxygen atom of the linker is hooked to Arg 705 of ASK1 protein.

### EXAMPLES

The following examples are given by way of illustration and therefore should not be construed to limit the scope of the present invention.

Temperatures are given in degree Celsius. The structures of final products, intermediates and starting materials are confirmed by standard analytical methods, spectroscopic characterization e.g., MS, NMR. Abbreviations used are those conventional in the art.

All starting materials, reagents, catalysts, building blocks, acids, bases, dehydrating agents and solvents utilized to synthesize the compounds of the present invention are either commercially available or can be produced by known organic synthesis methods in the art.

### Example 1

### General Procedure A:

Commercially available Boc-amino-PEG-amine (1 equivalent) was taken in 3 mL of pyridine-DICHLOROMETHANE-ACETONITRILE mixture (1:1:1). Succinic anhydride (1.1 equivalent) was then taken in the reaction mixture followed by stir at rt for 3.5 hours. The reaction was monitored by checking TLC and upon completion, the reaction mixture was diluted with CCl₄ and evaporated to dryness multiple times for the complete removal of pyridine to give the product. The product was carried forward for the next step without further purification. Using this procedure, we have synthesized compounds 2a, 2b, 2c, 2d, 2e, and 2f.

1H NMR of 2a: (400 MHz, Chloroform-d) δ 7.03 (s, 1H), 6.67 (s, 1H), 3.43 (dq, J=9.8, 5.7, 5.0 Hz, 6H), 3.32-3.24 (m, 2H), 2.73-2.66 (m, 2H), 2.55-2.41 (m, 2H), 1.45 (s, 9H).

1H NMR of 2b: (300 MHz, DMSO-d6) δ 3.72-3.24 (m, 14H), 2.78-2.39 (m, 2H), 1.42 (s, 9H).

1H NMR of 2d: (400 MHz, Chloroform-d) δ 5.27 (s, 1H), 4.72 (s, 1H), 3.64 (dd, J=12.0, 4.9 Hz, 18H), 3.53 (dt, J=9.0, 4.9 Hz, 4H), 3.44 (q, J=5.0 Hz, 2H), 3.30 (s, 2H), 2.66 (s, 2H), 2.54 (s, 2H), 1.43 (s, 9H).

1H NMR of 2e: 1H NMR (400 MHz, Chloroform-d) δ 7.04 (s, 1H), 5.16 (s, 1H), 3.64 (d, J=4.4 Hz, 10H), 3.63-3.58 (m, 9H), 3.56-3.49 (m, 4H), 3.42 (q, J=5.1 Hz, 2H), 3.28 (s, 2H), 2.99 (s, 1H), 2.65-2.60 (m, 2H), 2.55-2.49 (m, 2H), 1.42 (s, 9H).

### Example 2

Commercially available 3-fluoro phthalic anhydride (1 equivalent) and 3-aminopiperidine-2,6-dione hydrochloride (1.1 equivalent) were taken in AcOH. NaOAc (2 equivalent) was added to the reaction mixture and set to reflux for 12 hours. The reaction was monitored by checking TLC and upon completion, the reaction mixture was evaporated to dryness. The reaction mixture was diluted with 2 mL of milli-Q water and neutralized with Liq. NH₃ to pH 8 and extracted with EtOAc and finally washed with brine solution to give the product as an off-white solid. 1H NMR (400 MHz, DMSO-d6) δ 11.10 (s, 1H), 7.93-7.88 (m, 1H), 7.76-7.67 (m, 2H), 5.12 (dd, J=12.9, 5.4 Hz, 1H), 2.85 (ddd, J=17.1, 13.9, 5.5 Hz, 1H), 2.63-2.47 (m, 2H), 2.03 (d, J=12.4 Hz, 1H).

### Example 3

Commercially available compound methyl 6-amino picolinate (1 equivalent) was dissolved in methanol to give a light yellow solution, and then hydrazine hydrate (3 equivalent) was added slowly. The reaction system was heated to reflux for 12 hours. After the reaction was completed, the reaction solution was cooled to room temperature and filtered. The filter cake was washed with ethyl acetate and dried on a rotary evaporator under reduced pressure to produce the compound 7 (100% yield) as white solid and carried forward for the next step of synthesis. 1H NMR (600 MHz, DMSO-d6) δ 9.16 (s, 1H), 7.50 (t, J=7.7 Hz, 1H), 7.10 (d, J=7.1 Hz, 1H), 6.60 (d, J=8.2 Hz, 1H), 6.08 (s, 2H), 4.48 (s, 2H).

### Example 4

Compound 7 (1 equivalent) was added to dimethylformamide dimethyl acetal (500 mL). The system was refluxed with stirring at 110° C. for 12 hours. After the reaction was completed, the reaction solution was dried on a rotary evaporator under reduced pressure to obtain a crude product. To the crude product was added ethyl acetate (500 mL), stirred for 20 min at room temperature and filtered. The filter cake was dried to give compound 8 (82% yield) was obtained as a yellow solid. 1H NMR (400 MHz, CHLOROFORM-d) δ ppm 2.95 (s, 6H) 3.11 (d, J=6.27 Hz, 6H) 7.03 (dd, J=7.91, 0.88 Hz, 1H) 7.67 (t, J=7.78 Hz, 1H) 7.78 (dd, J=7.40, 0.88 Hz, 1H) 8.15 (s, 1H) 8.34 (s, 1H) 9.95 (s, 1H).

### Example 5

Compound 8 (1 equivalent) was dissolved in a mixture of acetonitrile, acetic acid and isopropyl amine (1.2 equivalent) was added. The system was stirred at 80° C. for 12 hours. After the reaction was completed, the reaction solution was allowed to stand, and filtered. The filtrate was dried on a rotary evaporator under reduced pressure, added with water, adjusted to pH 9-10 with dropwise addition of sodium hydroxide, and extracted with ethyl acetate. The organic phases were combined and evaporated on a rotary evaporator under reduced pressure until a large amount of yellow solid precipitated out, allowed to stand and filtered. The filter cake was washed with cold ethyl acetate and dried to obtain product compound 9 (98% purity) as a light yellow crystal. 1H NMR (400 MHz, DMSO-d6) δ 8.73 (s, 1H), 7.48 (dd, J=8.3, 7.4 Hz, 1H), 7.13 (dd, J=7.4, 0.8 Hz, 1H), 6.49 (dd, J=8.3, 0.8 Hz, 1H), 6.11 (s, 2H), 5.48 (hept, J=6.7 Hz, 1H), 1.39 (d, J=6.7 Hz, 6H).

### Example 6

Commercially available compound 2,4-difluoro-5-nitrobenzoic acid was taken in MeOH. NH₂NH₂·H₂O (2.5 eq.), oxalyl chloride (3 eq.) and a catalytic amount of DMF were added to the reaction mixture at ice-cold followed by stirring at rt for 1 hour. The solvent was then evaporated to dryness followed by the addition of the mixture of compound 6 in pyridine. The final reaction mixture was then stirred at rt for another 2 hours to produce a yellow mass. The yellow mass was then purified by column chromatography using 10% MeOH in CHCl₃ to give compound 12 as yellow solid (70% yield). 1H NMR (400 MHz, DMSO-d6) δ 11.08 (s, 1H), 8.84 (s, 1H), 8.56 (t, J=7.7 Hz, 1H), 8.15 (d, J=8.2 Hz, 1H), 8.02 (t, J=7.9 Hz, 1H), 7.94-7.85 (m, 2H), 5.57 (p, J=6.6 Hz, 1H), 1.40 (d, J=6.7 Hz, 6H).

### Example 7

Compound 12 was taken in MeOH. SnCl₂ was added to the reaction mixture with a catalytic amount of HCl. The reaction mixture was then refluxed for 3 hours. After the completion of the reaction, the reaction mixture was neutralised by aqueous NaOH and was then purified by column chromatography using 10% MeOH in CHCl₃ to give compound 13 as a yellow solid (70% yield). 1H NMR (400 MHz, Methanol-d4) δ 8.81 (s, 1H), 8.30 (d, J=8.1 Hz, 1H), 7.97 (t, J=7.8 Hz, 1H), 7.87-7.84 (m, 1H), 7.34-7.22 (m, 1H), 6.98 (t, J=10.7 Hz, 1H), 5.77-5.54 (m, 1H), 1.52 (d, J=6.6 Hz, 6H).

### Example 8

### General Procedure B:

Compound 2a (1.2 equivalent) was taken in DMF and HATU (1.2 equivalent) was added and the reaction mixture was allowed to stir at room temperature for 15 min to 30 min. Suitable substituted aliphatic or aromatic amine was added drop wise (1.2 equivalent) to the reaction mixture followed by DIPEA (3 equivalent) and the reaction mixture was stirred for 24 hours. The reaction was monitored by checking TLC. Upon completion, the reaction mixture was washed thoroughly with ice-cold water to remove DMF and extracted with EtOAc. Column chromatography (10% MeOH in CHCl₃) was performed to get compound 14 (78% yield) as colourless liquid. 1H NMR (400 MHz, Chloroform-d) δ 8.93 (d, J=17.7 Hz, 3H), 8.40-8.34 (m, 2H), 8.02 (d, J=7.6 Hz, 1H), 7.88 (t, J=8.0 Hz, 1H), 7.03-6.93 (m, 1H), 6.55 (s, 1H), 5.48 (hept, J=6.7 Hz, 1H), 4.98 (s, 1H), 3.55-3.43 (m, 6H), 3.29 (q, J=4.8 Hz, 2H), 2.76 (dd, J=7.9, 3.8 Hz, 2H), 2.66 (dd, J=7.7, 4.1 Hz, 2H), 1.57 (s, 3H), 1.56 (s, 3H), 1.42 (s, 9H).

### Example 9

### General Procedure C:

Compound 14 was taken in a mixture of 4M HCl in dioxane in dioxane (1:1). The reaction mixture was then stirred at rt for 2 hours. After checking the completion of the reaction, the reaction mixture was purified by column chromatography using 10% MeOH in CHCl₃ to give compound 15 as yellow solid (94% yield). 1H NMR (400 MHz, DMSO-d6) δ 9.92 (s, 1H), 8.82 (s, 1H), 8.16 (t, J=8.9 Hz, 2H), 8.00 (d, J=8.0 Hz, 1H), 7.93 (s, 1H), 7.86 (s, 1H), 7.48 (d, J=10.1 Hz, 1H), 5.61 (t, J=9.0 Hz, 1H), 3.47 (s, 1H), 3.34 (s, 4H), 3.30-3.28 (m, 3H), 3.16 (s, 3H), 2.59 (s, 3H), 2.38 (s, 2H), 1.95 (s, 1H), 1.39 (s, 6H).

### Example 10

### General Procedure D:

Compound 5 was added to the mixture of compound 15 in DMF. DIPEA was added to the reaction mixture and set to reflux for 12 hours. After completion of the reaction, the reaction mass was then purified by column chromatography using 10% MeOH in CHCl₃ to give compound 16 as yellow powdered solid (72% yield). 1H NMR (400 MHz, Chloroform-d) δ 9.35 (s, 1H), 9.04 (d, J=13.7 Hz, 1H), 8.84 (t, J=8.5 Hz, 1H), 8.43-8.35 (m, 3H), 8.03 (d, J=7.6 Hz, 1H), 7.90 (t, J=8.0 Hz, 1H), 7.51-7.45 (m, 1H), 7.09 (d, J=7.1 Hz, 1H), 6.95 (t, J=10.5 Hz, 1H), 6.86 (d, J=8.5 Hz, 1H), 6.72 (d, J=5.2 Hz, 2H), 5.49 (p, J=6.7 Hz, 1H), 5.17-5.08 (m, 1H), 3.73 (t, J=4.6 Hz, 2H), 3.62 (t, J=4.5 Hz, 2H), 3.57-3.51 (m, 2H), 3.43 (q, J=5.0 Hz, 2H), 2.84-2.74 (m, 7H), 2.17-2.09 (m, 1H), 1.56 (d, J=6.7 Hz, 6H).

### Example 11

Compound 2b (1.2 equivalent) was taken in DMF and HATU (1.2 equivalent) and allowed to stir at room temperature for 30 min. Compound 13 was then added drop wise (1 equivalent) to the reaction mixture followed by DIPEA (3 equivalent) and the reaction mixture was stirred for 24 hours. The reaction was monitored by checking TLC. Upon completion, the reaction mixture was washed thoroughly with ice-cold water to remove DMF and extracted with EtOAc. Column chromatography (10% MeOH in CHCl3) was performed to get compound 17 (74% yield) as colourless liquid. 1H NMR (600 MHz, Chloroform-d) δ 9.03 (s, 1H), 8.97 (d, J=13.5 Hz, 1H), 8.89 (t, J=8.4 Hz, 1H), 8.53 (s, 1H), 8.41 (d, J=8.3 Hz, 1H), 8.03-7.97 (m, 1H), 7.91 (t, J=8.0 Hz, 1H), 7.87 (s, 1H), 7.00 (t, J=10.4 Hz, 1H), 6.65 (s, 1H), 5.50 (p, J=6.7 Hz, 1H), 5.07 (s, 1H), 3.77-3.69 (m, 2H), 3.68-3.54 (m, 11H), 3.51-3.47 (m, 2H), 3.33 (s, 3H), 3.19 (qd, J=7.4, 4.2 Hz, 2H), 2.77 (d, J=6.3 Hz, 2H), 2.68 (s, 3H), 1.59 (d, J=6.7 Hz, 6H), 1.50-1.45 (m, 10H), 1.44 (s, 9H), 1.42 (s, 4H).

### Example 12

Compound 17 was taken in a mixture of 4M HCl in dioxane and dioxane (1:1). The reaction mixture was then stirred at rt for 2 hours. After checking the completion of the reaction the reaction mixture was purified by column chromatography using 10% MeOH in CHCl₃ to give compound 18 as yellow solid (94% yield).

### Example 13

Compound 18 was added to the mixture of compound 5 in DMF. DIPEA was added to the reaction mixture and set to reflux for 12 hours. After completion of the reaction, the reaction mass was then purified by column chromatography using 10% MeOH in CHCl₃ to give compound 19 as yellow powdered solid (71% yield).

### Example 14

Compound 2c (1.2 equivalent) was taken in DMF and HATU (1.2 equivalent) and allowed to stir at room temperature for 30 min. Compound 13 was then added drop wise (1 equivalent) to the reaction mixture followed by DIPEA (3 equivalent) and the reaction mixture was stirred for 24 hours. The reaction was monitored by checking TLC. Upon completion, the reaction mixture was washed thoroughly with ice-cold water to remove DMF and extracted with EtOAc. Column chromatography (10% MeOH in CHCl3) was performed to get compound 20 (72% yield) as a colourless liquid.

### Example 15

Compound 20 was taken in a mixture of 4M HCl in dioxane and dioxane (1:1). The reaction mixture was then stirred at rt for 2 hours. After checking the completion of the reaction the reaction mixture was purified by column chromatography using 10% MeOH in CHCl₃ to give compound 21 as yellow solid (92% yield).

### Example 16

Compound 21 was added to the mixture of compound 5 in DMF. DIPEA was added to the reaction mixture and set to reflux for 12 hours. After completion of the reaction, the reaction mass was then purified by column chromatography using 10% MeOH in CHCl₃ to give compound 22 as yellow powdered solid (70% yield).

### Example 17

Compound 2d (1.2 equivalent) was taken in DMF and HATU (1.2 equivalent) and allowed to stir at room temperature for 30 min. Compound 13 was then added drop wise (1 equivalent) to the reaction mixture followed by DIPEA (3 equivalent) and the reaction mixture was stirred for 24 hours. The reaction was monitored by checking TLC. Upon completion, the reaction mixture was washed thoroughly with ice-cold water to remove DMF and extracted with EtOAc. Column chromatography (10% MeOH in CHCl₃) was performed to get compound 23 (70% yield) as a colourless liquid.

### Example 18

Compound 23 was taken in a mixture of 4M HCl in dioxane and dioxane (1:1). The reaction mixture was then stirred at rt for 2 hours. After checking the completion of the reaction the reaction mixture was purified by column chromatography using 10% MeOH in CHCl₃ to give compound 24 as yellow solid (90% yield).

### Example 19

Compound 24 was added to the mixture of compound 5 in DMF. DIPEA was added to the reaction mixture and set to reflux for 12 hours. After completion of the reaction, the reaction mass was then purified by column chromatography using 10% MeOH in CHCl₃ to give compound 25 as yellow powdered solid (70% yield).

### Example 20

Compound 2e (1.2 equivalent) was taken in DMF and HATU (1.2 equivalent) and allowed to stir at room temperature for 30 min. Compound 13 was then added drop wise (1 equivalent) to the reaction mixture followed by DIPEA (3 equivalent) and the reaction mixture was stirred for 24 hours. The reaction was monitored by checking TLC. Upon completion, the reaction mixture was washed thoroughly with ice-cold water to remove DMF and extracted with EtOAc. Column chromatography (10% MeOH in CHCl₃) was performed to get compound 26 (70% yield) as colourless liquid. 1H NMR (400 MHz, Chloroform-d) δ 9.40 (s, 1H), 8.96 (d, J=13.4 Hz, 1H), 8.87 (t, J=8.5 Hz, 1H), 8.38 (d, J=9.0 Hz, 1H), 8.35 (s, 1H), 8.01 (d, J=7.7 Hz, 1H), 7.88 (t, J=8.0 Hz, 1H), 7.49 (s, 1H), 7.03-6.92 (m, 1H), 5.48 (p, J=6.7 Hz, 1H), 5.32 (t, J=4.7 Hz, 1H), 3.63 (s, 8H), 3.60 (dt, J=4.8, 2.2 Hz, 8H), 3.57-3.52 (m, 2H), 3.50 (t, J=5.1 Hz, 2H), 3.45 (q, J=5.0 Hz, 2H), 3.33-3.23 (m, 2H), 2.73 (dd, J=7.8, 3.8 Hz, 2H), 2.66 (dd, J=7.9, 3.8 Hz, 2H), 1.55 (d, J=6.7 Hz, 6H), 1.40 (s, 9H).

### Example 21

Compound 26 was taken in a mixture of 4M HCl in dioxane and dioxane (1:1). The reaction mixture was then stirred at rt for 2 hours. After checking the completion of the reaction the reaction mixture was purified by column chromatography using 10% MeOH in CHCl₃ to give compound 27 as a yellow solid (91% yield).

### Example 22

Compound 27 was added to the mixture of compound 5 in DMF. DIPEA was added to the reaction mixture and set to reflux for 12 hours. After completion of the reaction, the reaction mass was then purified by column chromatography using 10% MeOH in CHCl₃ to give compound 28 as yellow powdered solid (70% yield). 1H NMR (400 MHz, CDCl3) δ 9.33 (s, 1H), 8.86-8.95 (m, 2H), 8.38 (d, J=8.28 Hz, 1H), 8.35 (s, 1H), 8.02 (d, J=7.64 Hz, 1H), 7.88 (t, J=8.08 Hz, 1H), 7.12 (s, 1H), 6.97 (t, J=10.48 Hz, 1H), 5.44-5.54 (m, 1H), 5.12 (s, 1H), 3.59-3.63 (m, 18H), 3.55 (t, J=5.08 Hz, 2H), 3.51 (t, J=5.16 Hz, 2H), 3.46 (t, J=5.12 Hz, 1H), 3.27-3.28 (m, 2H), 2.74 (m, 2H), 2.65 (m, 2H), 2.46 (s, 2H), 1.57 (s, 3H), 1.55 (s, 3H), 1.41 (s, 9H).

### Example 23

Compound 2f (1.2 equivalent) was taken in DMF and HATU (1.2 equivalent) and allowed to stir at room temperature for 30 min. Compound 13 was then added drop wise (1 equivalent) to the reaction mixture followed by DIPEA (3 equivalent) and the reaction mixture was stirred for 24 hours. The reaction was monitored by checking TLC. Upon completion, the reaction mixture was washed thoroughly with ice-cold water to remove DMF and extracted with EtOAc. Column chromatography (10% MeOH in CHCl3) was performed to get compound 29 (70% yield) as colourless liquid. 1H NMR (400 MHz, Chloroform-d) δ 9.33 (s, 1H), 9.00-8.83 (m, 2H), 8.38 (d, J=8.3 Hz, 1H), 8.35 (s, 1H), 8.02 (d, J=7.6 Hz, 1H), 7.89 (t, J=8.0 Hz, 1H), 7.13 (s, 1H), 7.02-6.93 (m, 1H), 5.49 (p, J=6.7 Hz, 1H), 5.14 (s, 1H), 3.64 (d, J=2.9 Hz, 10H), 3.62-3.59 (m, 8H), 3.57-3.54 (m, 2H), 3.51 (t, J=5.1 Hz, 2H), 3.46 (q, J=5.0 Hz, 2H), 3.28 (d, J=5.0 Hz, 2H), 2.75 (dd, J=8.0, 3.9 Hz, 2H), 2.66 (dd, J=7.9, 4.0 Hz, 2H), 2.46 (s, 2H), 1.57 (s, 3H), 1.42 (s, 9H).

### Example 24

Compound 29 was taken in a mixture of 4M HCl in dioxane and dioxane (1:1). The reaction mixture was then stirred at rt for 2 hours. After checking the completion of the reaction the reaction mixture was purified by column chromatography using 10% MeOH in CHCl₃ to give compound 30 as a yellow solid (91% yield).

### Example 25

Compound 30 was added to the mixture of compound 5 in DMF. DIPEA was added to the reaction mixture and set to reflux for 12 hours. After completion of the reaction, the reaction mass was then purified by column chromatography using 10% MeOH in CHCl₃ to give compound 31 as a yellow powdered solid (70% yield). 1H NMR (400 MHz, CDCl3) δ 9.29 (s, 1H), 8.97 (s, 1H), 8.93 (s, 1H), 8.88 (t, J=7.96 Hz, 1H), 8.38 (d, J=8.36 Hz, 1H), 8.37 (s, 1H), 8.02 (d, J=7.64 Hz, 1H), 7.89 (t, J=8.04 Hz, 1H), 7.45 (t, J=8.08 Hz, 1H), 7.31 (bs, 1H), 7.06 (d, J=7.08 Hz, 1H), 6.95 (t, J=10.48 Hz, 1H), 6.87 (d, J=8.52 Hz, 1H), 6.49 (t, J=5.40 Hz, 1H), 5.44-5.54 (m, 1H), 4.89-4.93 (m, 1H), 3.70 (t, J=5.20 Hz, 2H), 3.63 (t, J=10.04 Hz, 20H), 3.55 (t, J=5.00 Hz, 2H), 3.46 (t, J=4.96 Hz, 2H), 3.43 (t, J=5.16 Hz, 2H), 2.71-2.84 (m, 4H), 2.64-2.67 (m, 2H), 2.09-2.14 (m, 2H), 1.57 (s, 3H), 1.56 (s, 3H).

### Example 26

Compound 5 (1 equivalent) which was previously synthesized by general procedure B in example 2 and Boc-amino-alkylamine (1.5 equivalent) was taken in dimethyl sulfoxide under Ar atmosphere. DIPEA (3.5 equivalent) was added to the reaction mixture and set to reflux for 12 hours. The reaction was monitored by checking TLC. Upon completion, the reaction mixture was diluted with cold water and EtOAc. Then EtOAc part was washed thoroughly with ice-cold water to remove dimethyl sulfoxide and the organic part was concentrated in rota vapour under reduced pressure to get the crude product. Then Column chromatography was performed to get the pure product to produce compound 32 (70% yield) as a yellowish solid. This pure product was carried forward to the next step.

### Example 27

Compound 32 (1 equivalent) was taken in DICHLOROMETHANE and 4 M HCl in dioxane was added to this reaction mixture. The reaction mixture was stirred for 2 hours at room temperature. The reaction was monitored by checking TLC. Upon completion, excess 4M HCl in dioxane was evaporated in rota vapour. The reaction mixture was 10% MeOH—CHCl₃ and water. Then the organic part was separated and concentrated in rota vapour to get the crude product. This crude product was purified by silica get column purification to get the pure product compound 33 as an off-white solid (96% yield).

### Example 28

Compound 34 (80% yield) was synthesized by general procedure A in example 1.

### Example 29

Acid compound 34 (1.2 equivalent) was taken in DMF (1-2 mL) and HATU (1.2 equivalent) was added and the reaction mixture was allowed to stir at room temperature for 15 min to 30 min. Suitable substituted compound 13 (1 equivalent) was added to the reaction mixture followed by DIPEA (2.5-3 equivalent) and the reaction mixture was stirred for another 2-3 days. The reaction was monitored by checking TLC. Upon completion, the reaction mixture was washed thoroughly with ice-cold water to remove DMF and extracted with EtOAc. Column chromatography was performed to get the pure product as a yellowish solid with 85% yields. ¹H NMR (400 MHz, Chloroform-d) δ 8.96 (d, J=13.7 Hz, 1H), 8.86 (s, 1H), 8.80 (dd, J=18.5, 10.0 Hz, 2H), 8.35 (d, J=4.4 Hz, 2H), 8.00 (d, J=7.4 Hz, 1H), 7.87 (t, J=7.9 Hz, 1H), 7.45-7.39 (m, 1H), 7.01 (d, J=7.1 Hz, 1H), 6.93 (t, J=10.5 Hz, 1H), 6.81 (d, J=8.5 Hz, 1H), 6.29 (d, J=5.1 Hz, 1H), 6.18 (t, J=5.4 Hz, 1H), 5.48-5.44 (m, 1H), 4.91 (td, J=8.0, 6.8, 3.7 Hz, 1H), 3.28 (q, J=6.3 Hz, 2H), 3.25-3.19 (m, 2H), 2.78-2.71 (m, 4H), 2.61 (dd, J=7.3, 4.1 Hz, 2H), 1.68-1.62 (m, 2H), 1.57 (s, 2H), 1.54 (s, 6H), 1.50 (d, J=6.8 Hz, 2H), 1.44 (d, J=6.7 Hz, 2H).

### Example 30

N-boc amino butanoic acid (1.2 equivalent) was taken in DMF (1-2 mL) and HATU (1.2 equivalent) was added and the reaction mixture was allowed to stir at room temperature for 30 min. Then compound 13 (1 equivalent) was added to the reaction mixture followed by DIPEA (2.5-3 equivalent) and the reaction mixture was stirred for another 12 hours. The reaction was monitored by checking TLC. Upon completion, the reaction mixture was washed thoroughly with ice-cold water to remove DMF and extracted with EtOAc. Column chromatography was performed to get the pure product as a colourless liquid compound 36 with 85% yield.

### Example 31

Compound 36 (1 equivalent) was taken in dichloromethane and 4 M HCl in dioxane was added to this reaction mixture. The reaction mixture was stirred for 2 hours at room temperature. The reaction was monitored by checking TLC. Upon completion, excess 4M HCl in dioxane was evaporated in rota vapour. The reaction mixture was 10% MeOH—CHCl₃ and water. Then the organic part was separated and concentrated in rota vapour to get the crude product. This crude product was purified by silica get column purification to get the pure product compound 37 as an off-white solid with 95% yield.

### Example 32

Compound 37 (1 equivalent) was taken in dimethyl sulfoxide under Ar atmosphere then DIPEA (3.5 equivalent) was added to the reaction mixture at room temperature and stirred the reaction mixture for 30 minutes. Then compound 5 was added to this reaction mixture and stirred this reaction for 12 hours at room temperature. The reaction was monitored by checking TLC. Upon completion, the reaction mixture was diluted with cold water and EtOAc. Then EtOAc part was washed thoroughly with ice-cold water to remove dimethyl sulfoxide and the organic part was concentrated in rotavapour under reduced pressure to get the crude product. Then Column chromatography was performed to get the pure product to produce compound 38 with 80% yield as a yellowish solid. ¹H NMR (400 MHz, Chloroform-d) δ 8.99 (d, J=13.3 Hz, 1H), 8.78-8.69 (m, 1H), 8.36 (s, 1H), 8.33 (d, J=8.3 Hz, 1H), 7.96 (t, J=8.3 Hz, 1H), 7.84 (t, J=7.9 Hz, 1H), 7.44 (t, J=7.5 Hz, 1H), 7.03 (t, J=8.7 Hz, 1H), 6.92 (q, J=7.8, 5.5 Hz, 2H), 6.31 (t, J=5.1 Hz, 1H), 5.49-5.38 (m, 1H), 4.93 (dd, J=12.2, 5.5 Hz, 1H), 3.44-3.29 (m, 2H), 2.74 (t, J=10.8 Hz, 2H), 2.58 (t, J=6.6 Hz, 2H), 2.06 (dt, J=10.5, 5.2 Hz, 4H), 1.54 (d, J=6.2 Hz, 6H).

### Example 33

N-Boc amino pentatonic acid (1.2 equivalent) was taken in DMF (1-2 mL) and HATU (1.2 equivalent) was added and the reaction mixture was allowed to stir at room temperature for 30 min. Then compound 13 (1 equivalent) was added to the reaction mixture followed by DIPEA (3.5 equivalent) and the reaction mixture was stirred for another 12 hours. The reaction was monitored by checking TLC. Upon completion, the reaction mixture was washed thoroughly with ice-cold water to remove DMF and extracted with EtOAc. Column chromatography was performed to get the pure product as a colourless liquid compound 39 with 85% yield.

### Example 34

Compound 39 (1 equivalent) was taken in dichloromethane and 4 M HCl in dioxane was added to this reaction mixture. The reaction mixture was stirred for 2 hours at room temperature. The reaction was monitored by checking TLC. Upon completion, excess 4M HCl in dioxane was evaporated in rota vapour. The reaction mixture was 10% MeOH—CHCl₃ and water. Then the organic part was separated and concentrated in rotavapour to get the crude product. This crude product was purified by silica get column purification to get the pure product compound 40 as an off-white solid 95% yield.

### Example 35

Compound 40 (1 equivalent) was taken in DMSO under Ar atmosphere then DIPEA (3.5 equivalent) was added to the reaction mixture at room temperature and stirred the reaction mixture for 30 minutes. Then compound 5 was added to this reaction mixture and stirred this reaction for 12 hours at room temperature. The reaction was monitored by checking TLC. Upon completion, the reaction mixture was diluted with cold water and EtOAc. Then EtOAc part was washed thoroughly with ice-cold water to remove DMSO and the organic part was concentrated in rotavapour under reduced pressure to get the crude product. Then column chromatography was performed to get the pure product to produce compound 41 as a yellowish solid 70% yield. ¹H NMR (400 MHz, Chloroform-d) δ 8.48 (q, J=7.7, 7.3 Hz, 1H), 8.35 (s, 1H), 8.33-8.27 (m, 1H), 7.87 (s, 2H), 7.42 (t, J=7.8 Hz, 1H), 7.25 (s, 2H), 6.97 (dd, J=23.4, 8.7 Hz, 2H), 6.85 (s, 1H), 5.45 (ddt, J=13.0, 9.8, 5.0 Hz, 1H), 4.89-4.74 (m, 1H), 3.30-3.28 (m, 4H), 2.78 (d, J=13.8 Hz, 1H), 2.69 (q, J=10.9, 10.2 Hz, 2H), 2.42 (t, J=6.9 Hz, 2H), 2.04 (ddt, J=10.1, 5.7, 3.1 Hz, 1H), 1.84-1.74 (m, 2H), 1.74-1.64 (m, 2H), 1.50 (dd, J=6.7, 3.0 Hz, 6H).

### Example 36

N-Boc amino hexanoic acid (1.2 equivalent) was taken in DMF (1 mL) and HATU (1.2 equivalent) was added and the reaction mixture was allowed to stir at room temperature for 30 min. Then compound 13 (1 equivalent) was added to the reaction mixture followed by DIPEA (3.5 equivalent) and the reaction mixture was stirred for another 12 hours. The reaction was monitored by checking TLC. Upon completion, the reaction mixture was washed thoroughly with ice-cold water to remove DMF and extracted with EtOAc. Column chromatography was performed to get the pure product as a colourless liquid compound 42 with 85% yield. ¹H NMR (400 MHz, Chloroform-d) δ 8.92 (d, J=13.5 Hz, 1H), 8.84 (t, J=8.5 Hz, 1H), 8.38 (d, J=8.5 Hz, 2H), 8.00 (d, J=7.7 Hz, 1H), 7.88 (t, J=8.0 Hz, 1H), 7.76 (s, 1H), 7.03-6.95 (m, 1H), 5.48 (p, J=6.7 Hz, 1H), 4.62 (s, 1H), 3.12 (q, J=6.2 Hz, 2H), 2.44 (t, J=7.4 Hz, 2H), 1.91 (s, 2H), 1.76 (p, J=7.5 Hz, 2H), 1.58 (s, 3H), 1.56 (s, 3H), 1.51 (q, J=7.0 Hz, 2H), 1.42 (s, 9H).

### Example 37

Compound 42 (1 equivalent) was taken in dichloromethane and 4 M HCl in dioxane was added to this reaction mixture. The reaction mixture was stirred for 2 hours at room temperature. The reaction was monitored by checking TLC. Upon completion, excess 4M HCl in dioxane was evaporated in rota vapour. The reaction mixture was 10% MeOH—CHCl₃ and water. Then the organic part was separated and concentrated in rota vapour to get the crude product. This crude product was purified by silica get column purification to get the pure product compound 43 as an off-white solid with 96% yield.

### Example 38

Compound 43 (1 equivalent) was taken in DMSO under Ar atmosphere then DIPEA (3.5 equivalent) was added to the reaction mixture at room temperature and stirred the reaction mixture for 30 minutes. Then compound 5 was added to this reaction mixture and stirred this reaction for 12 hours at room temperature. The reaction was monitored by checking TLC. Upon completion, the reaction mixture was diluted with cold water and EtOAc. Then EtOAc part was washed thoroughly with ice-cold water to remove DMSO and the organic part was concentrated in rotavapour under reduced pressure to get the crude product. Then Column chromatography was performed to get the pure product to produce compound 44 as a light yellowish solid with 75% yields. ¹H NMR (400 MHz, Chloroform-d) δ 8.97-8.88 (m, 1H), 8.87-8.77 (m, 1H), 8.37 (d, J=8.6 Hz, 3H), 8.00 (d, J=7.6 Hz, 1H), 7.87 (t, J=7.9 Hz, 1H), 7.71 (d, J=11.7 Hz, 1H), 7.46 (t, J=7.8 Hz, 1H), 7.05 (d, J=7.1 Hz, 1H), 6.97 (t, J=10.4 Hz, 1H), 6.86 (d, J=8.5 Hz, 1H), 6.21 (s, 1H), 5.46 (dt, J=12.3, 6.0 Hz, 1H), 4.90 (dd, J=11.9, 5.4 Hz, 1H), 3.27 (s, 1H), 2.89-2.80 (m, 1H), 2.80-2.67 (m, 1H), 2.45 (t, J=7.2 Hz, 2H), 2.23 (s, 1H), 2.15-2.06 (m, 1H), 1.89 (s, 2H), 1.82-1.76 (m, 2H), 1.73-1.65 (m, 2H), 1.56 (d, J=6.6 Hz, 6H), 1.54-1.46 (m, 2H).

### Example 39

N-Boc amino heptanoic acid (1.2 equivalent) was taken in DMF (1 mL) and HATU (1.2 equivalent) was added and the reaction mixture was allowed to stir at room temperature for 30 min. Then compound 13 (1 equivalent) was added to the reaction mixture followed by DIPEA (3.5 equivalent) and the reaction mixture was stirred for another 12 hours. The reaction was monitored by checking TLC. Upon completion, the reaction mixture was washed thoroughly with ice-cold water to remove DMF and extracted with EtOAc. Column chromatography was performed to get the pure product as a colourless liquid compound 45 with 85% yield.

### Example 40

Compound 45 (1 equivalent) was taken in dichloromethane and 4 M HCl in dioxane was added to this reaction mixture. The reaction mixture was stirred for 2 hours at room temperature. The reaction was monitored by checking TLC. Upon completion, excess 4M HCl in dioxane was evaporated in rota vapour. The reaction mixture was 10% MeOH—CHCl₃ and water. Then the organic part was separated and concentrated in rota vapour to get the crude product. This crude product was purified by silica get column purification to get the pure product compound 46 as an off-white solid with 96% yield.

### Example 41

Compound 46 (1 equivalent) was taken in DMSO under Ar atmosphere then DIPEA (3.5 equivalent) was added to the reaction mixture at room temperature and stirred the reaction mixture for 30 minutes. Then compound 5 was added to this reaction mixture and stirred this reaction for 12 hours at room temperature. The reaction was monitored by checking TLC. Upon completion, the reaction mixture was diluted with cold water and EtOAc. Then EtOAc part was washed thoroughly with ice-cold water to remove DMSO and the organic part was concentrated in rotavapour under reduced pressure to get the crude product. Then Column chromatography was performed to get the pure product to produce compound 47 as a light yellowish solid with 80% yield. ¹H NMR (400 MHz, Chloroform-d) δ 8.99-8.84 (m, 2H), 8.39 (d, J=8.2 Hz, 1H), 8.35 (s, 1H), 8.23 (s, 1H), 8.02 (d, J=7.6 Hz, 1H), 7.89 (t, J=8.0 Hz, 1H), 7.56-7.44 (m, 2H), 7.06 (d, J=7.1 Hz, 1H), 6.99 (t, J=10.5 Hz, 1H), 6.87 (d, J=8.5 Hz, 1H), 6.21 (t, J=4.5 Hz, 1H), 5.51-5.43 (m, 1H), 4.91 (t, J=6.1 Hz, 1H), 3.62 (s, 2H), 3.27 (q, J=6.5 Hz, 2H), 2.87-2.73 (m, 2H), 2.42 (t, J=7.3 Hz, 2H), 1.73 (d, J=7.2 Hz, 4H), 1.56 (d, J=6.7 Hz, 6H), 1.48-1.42 (m, 4H).

### Example 42

N-boc amino octanoic acid (1.2 equivalent) was taken in DMF (1 mL) and HATU (1.2 equivalent) was added and the reaction mixture was allowed to stir at room temperature for 30 min. Then compound 13 (1 equivalent) was added to the reaction mixture followed by DIPEA (3.5 equivalent) and the reaction mixture was stirred for another 12 hours. The reaction was monitored by checking TLC. Upon completion, the reaction mixture was washed thoroughly with ice-cold water to remove DMF and extracted with EtOAc. Column chromatography was performed to get the pure product as a colourless liquid compound 48 with 90% yield.

### Example 43

Compound 48 (1 equivalent) was taken in dichloromethane and 4 M HCl in dioxane was added to this reaction mixture. The reaction mixture was stirred for 2 hours at room temperature. The reaction was monitored by checking TLC. Upon completion, excess 4M HCl in dioxane was evaporated in rota vapour. The reaction mixture was 10% MeOH—CHCl₃ and water. Then the organic part was separated and concentrated in rota vapour to get the crude product. This crude product was purified by silica get column purification to get the pure product compound 49 as an off-white solid with an 85% yield.

### Example 44

Compound 49 (1 equivalent) was taken in DMSO under Ar atmosphere then DIPEA (3.5 equivalent) was added to the reaction mixture at room temperature and stirred the reaction mixture for 30 minutes. Then compound 5 was added to this reaction mixture and stirred this reaction for 12 hours at room temperature. The reaction was monitored by checking TLC. Upon completion, the reaction mixture was diluted with cold water and EtOAc. Then EtOAc part was washed thoroughly with ice-cold water to remove DMSO and the organic part was concentrated in rotavapour under reduced pressure to get the crude product. Then Column chromatography was performed to get the pure product to produce Compound 50 as a light yellowish solid with a 70% yield. ¹H NMR (400 MHz, Chloroform-d) δ 8.96-8.85 (m, 2H), 8.40-8.37 (m, 1H), 8.37 (s, 1H), 8.30 (s, 1H), 8.05-8.00 (m, 1H), 7.90 (t, J=8.0 Hz, 1H), 7.54 (s, 1H), 7.51-7.45 (m, 1H), 7.07 (d, J=6.8 Hz, 1H), 7.03-6.96 (m, 1H), 6.87 (d, J=8.5 Hz, 1H), 6.22 (t, J=5.5 Hz, 1H), 5.48 (p, J=6.7 Hz, 1H), 4.91 (dd, J=12.1, 5.4 Hz, 1H), 3.26 (q, J=6.8 Hz, 2H), 2.91-2.67 (m, 3H), 2.42 (t, J=7.4 Hz, 2H), 2.16-2.09 (m, 1H), 1.76-1.72 (m, 2H), 1.69-1.64 (m, 2H), 1.57 (d, J=6.7 Hz, 6H), 1.41 (s, 6H).

### Example 45

N-Boc amino nonanoic acid (1.2 equivalent) was taken in DMF (1 mL) and HATU (1.2 equivalent) was added and the reaction mixture was allowed to stir at room temperature for 30 min. Then compound 13 (1 equivalent) was added to the reaction mixture followed by DIPEA (3.5 equivalent) and the reaction mixture was stirred for another 12 hours. The reaction was monitored by checking TLC. Upon completion, the reaction mixture was washed thoroughly with ice-cold water to remove DMF and extracted with EtOAc. Column chromatography was performed to get the pure product as a colourless liquid compound 51 with 84% yield.

### Example 46

Compound 51 (1 equivalent) was taken in dichloromethane and 4 M HCl in dioxane was added to this reaction mixture. The reaction mixture was stirred for 2 hours at room temperature. The reaction was monitored by checking TLC. Upon completion, excess 4M HCl in dioxane was evaporated in rota vapour. The reaction mixture was 10% MeOH—CHCl₃ and water. Then the organic part was separated and concentrated in rotavapour to get the crude product. This crude product was purified by silica get column purification to get the pure product compound 52 as an off-white solid with 96% yield.

### Example 47

Compound 52 (1 equivalent) was taken in DMSO under Ar atmosphere then DIPEA (3.5 equivalent) was added to the reaction mixture at room temperature and stirred the reaction mixture for 30 minutes. Then compound 5 was added to this reaction mixture and stirred this reaction for 12 hours at room temperature. The reaction was monitored by checking TLC. Upon completion, the reaction mixture was diluted with cold water and EtOAc. Then EtOAc part was washed thoroughly with ice-cold water to remove DMSO and the organic part was concentrated in rotavapour under reduced pressure to get the crude product. Then Column chromatography was performed to get the pure product to produce compound 53 as a light yellowish solid with 70% yields. ¹H NMR (400 MHz, Chloroform-d) δ 8.90 (d, J=13.1 Hz, 2H), 8.37 (d, J=15.6 Hz, 2H), 8.25 (s, 1H), 8.03 (s, 1H), 7.89 (d, J=12.1 Hz, 1H), 7.46 (d, J=9.7 Hz, 2H), 7.06 (s, 1H), 7.03-6.94 (m, 1H), 6.90-6.83 (m, 1H), 6.21 (s, 1H), 5.52-5.42 (m, 1H), 4.94-4.86 (m, 1H), 3.25 (s, 2H), 2.78 (td, J=34.5, 33.0, 15.8 Hz, 3H), 2.60 (s, 2H), 2.40 (s, 2H), 2.12 (d, J=12.3 Hz, 1H), 1.72 (t, J=27.9 Hz, 7H), 1.56 (s, 6H), 1.38 (d, J=17.7 Hz, 8H).

### Example 48

N-Boc amino decanoic acid (1.2 equivalent) was taken in DMF (1 mL) and HATU (1.2 equivalent) was added and the reaction mixture was allowed to stir at room temperature for 30 min. Then compound 13 (1 equivalent) was added to the reaction mixture followed by DIPEA (3.5 equivalent) and the reaction mixture was stirred for another 12 hours. The reaction was monitored by checking TLC. Upon completion, the reaction mixture was washed thoroughly with ice-cold water to remove DMF and extracted with EtOAc. Column chromatography was performed to get the pure compound 54 as a colourless liquid.

### Example 49

Compound 54 (1 equivalent) was taken in dichloromethane and 4 M HCl in dioxane was added to this reaction mixture. The reaction mixture was stirred for 2 hours at room temperature. The reaction was monitored by checking TLC. Upon completion, excess 4M HCl in dioxane was evaporated in rota vapour. The reaction mixture was 10% MeOH—CHCl₃ and water. Then the organic part was separated and concentrated in rota vapour to get the crude product. This crude product was purified by silica get column purification to get the pure product compound 55 as an off-white solid with 94% yield.

### Example 50

Compound 55 (1 equivalent) was taken in DMSO under Ar atmosphere then DIPEA (3.5 equivalent) was added to the reaction mixture at room temperature and stirred the reaction mixture for 30 minutes. Then compound 5 was added to this reaction mixture and stirred this reaction for 12 hours at room temperature. The reaction was monitored by checking TLC. Upon completion, the reaction mixture was diluted with cold water and EtOAc. Then EtOAc part was washed thoroughly with ice-cold water to remove DMSO and the organic part was concentrated in rotavapour under reduced pressure to get the crude product. Then Column chromatography was performed to get the pure product to produce Compound 56 as a light yellowish solid with a 73% yield. ¹H NMR (400 MHz, Chloroform-d) δ 8.90 (d, J=13.6 Hz, 2H), 8.38 (d, J=8.3 Hz, 1H), 8.35 (s, 1H), 8.01 (d, J=7.6 Hz, 1H), 7.88 (t, J=8.0 Hz, 1H), 7.53-7.43 (m, 2H), 7.05 (d, J=7.1 Hz, 1H), 6.98 (t, J=10.5 Hz, 1H), 6.86 (d, J=8.5 Hz, 1H), 6.21 (t, J=5.4 Hz, 1H), 5.47 (p, J=6.7 Hz, 1H), 4.91 (t, J=6.1 Hz, 1H), 3.24 (q, J=6.7 Hz, 2H), 2.89-2.66 (m, 3H), 2.60 (s, 1H), 2.41 (t, J=7.5 Hz, 2H), 2.11 (dd, J=7.9, 5.2 Hz, 1H), 1.76-1.68 (m, 2H), 1.67-1.60 (m, 2H), 1.56 (d, J=6.7 Hz, 6H), 1.43-1.28 (m, 10H).

### Example 51

3-(2-((tert-butoxycarbonyl)amino)ethoxy)propanoic acid (1.2 equivalent) was taken in DMF (1 mL) and HATU (1.2 equivalent) was added and the reaction mixture was allowed to stir at room temperature for 30 min. Then commercially available compound (2S,4R)-1-((R)-2-amino-3,3-dimethylbutanoyl)-4-hydroxy-N-(4-(4-methylthiazol-5-yl)benzyl)pyrrolidine-2-carboxamide hydrochloride (1 equivalent) was added to the reaction mixture followed by DIPEA (3.5 equivalent) and the reaction mixture were stirred for another 12 hours. The reaction was monitored by checking TLC. Upon completion, the reaction mixture was washed thoroughly with ice-cold water to remove DMF and extracted with EtOAc. Column chromatography was performed to get compound 58 as a colourless semi-solid (72% yield).

### Example 52

Compound 58 was taken in a mixture of 4M HCl in dioxane and dioxane (1:1). The reaction mixture was then stirred at rt for 2 hours. After checking the completion of the reaction the reaction mixture was purified by column chromatography using 10% MeOH in CHCl₃ to give compound 59 a white solid (98% yield). 1H NMR (600 MHz, Chloroform-d) δ 8.67 (s, 1H), 8.55-8.46 (m, 1H), 7.37-7.32 (m, 4H), 7.04 (d, J=8.8 Hz, 1H), 4.79-4.74 (m, 1H), 4.53 (d, J=9.0 Hz, 1H), 4.51 (s, 1H), 4.44 (td, J=15.8, 15.3, 5.5 Hz, 2H), 4.01 (d, J=10.8 Hz, 1H), 3.81-3.77 (m, 1H), 3.75-3.60 (m, 6H), 3.13 (s, 3H), 2.65-2.60 (m, 1H), 2.50 (s, 3H), 2.44 (d, J=13.7 Hz, 1H), 2.37-2.31 (m, 1H), 2.15-2.09 (m, 1H), 1.03 (s, 9H).

### Example 53

Compound 59 was used for the next step of synthesis following Example 1, General Procedure 1 to produce compound 60 as a white solid (78% yield).

The present invention relates to the compound of structure I or a salt thereof:

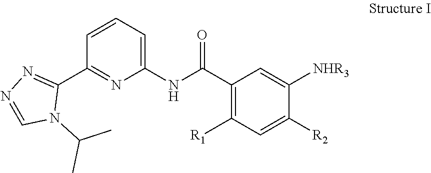

The compound of the present invention has the structure of Structure I as depicted in the table below:

Structure-Based Analogues (Table 1)

| Entry | R₁ | R₂ | R₃ | Structure |
|---|---|---|---|---|
| 1 | F | F | 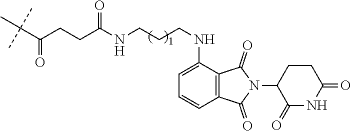 | 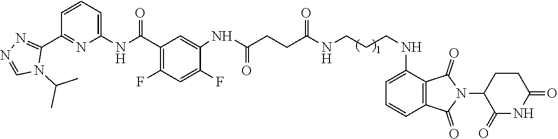 |
| 2 | F | F | 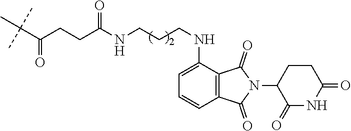 | 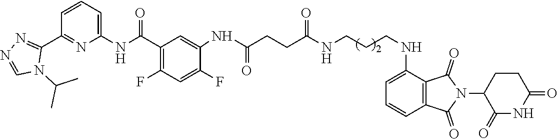 |
| 3 | F | F | 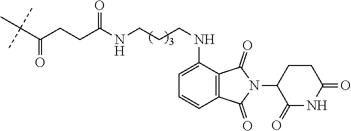 | 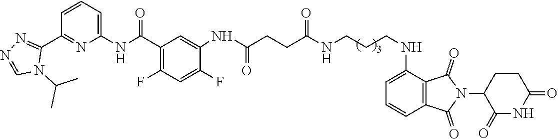 |
| 4 | F | F | 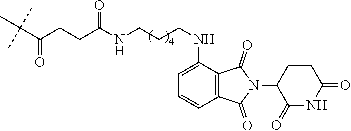 | 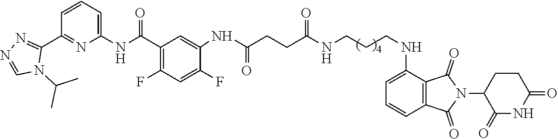 |
| 5 | F | F | 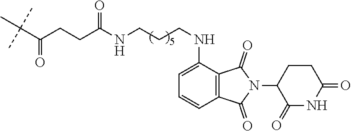 | 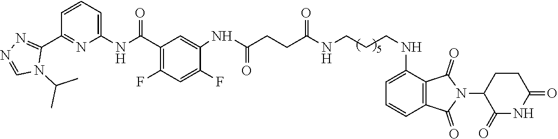 |
| 6 | F | F | 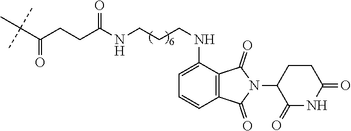 | 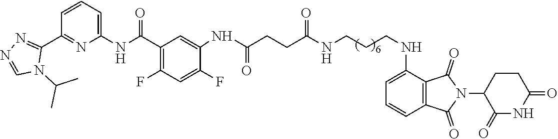 |
| 7 | F | F | 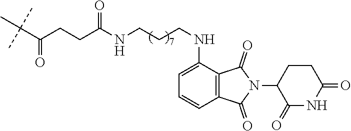 | 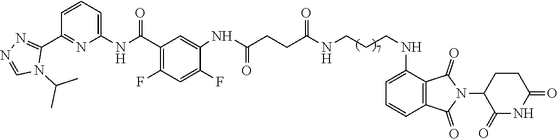 |
| 8 | F | F | 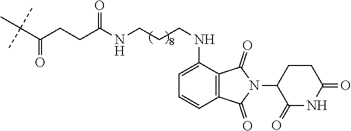 | 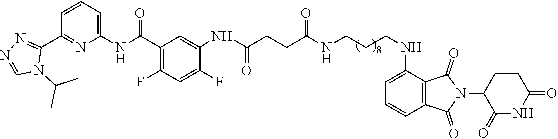 |
| 9 | F | F | 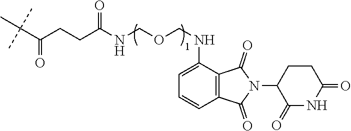 | 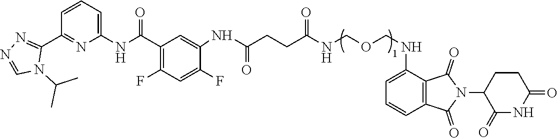 |
| 10 | F | F | 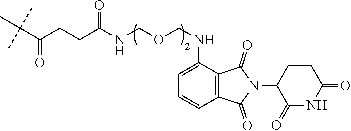 | 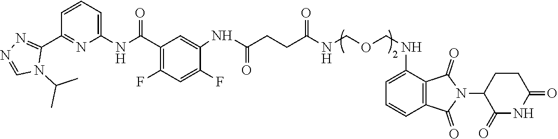 |
| 11 | F | F | 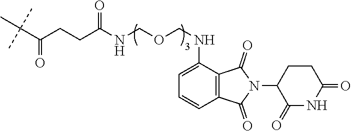 | 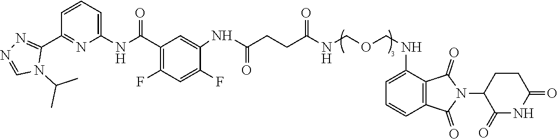 |
| 12 | F | F | 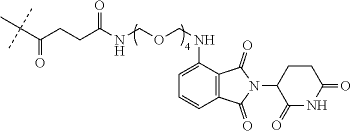 | 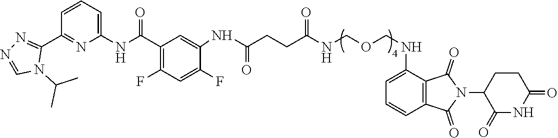 |
| 13 | F | F | 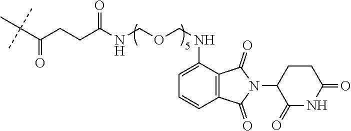 | 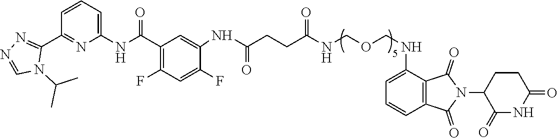 |
| 14 | F | F | 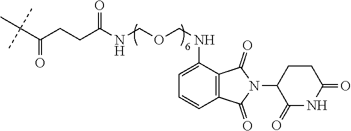 | 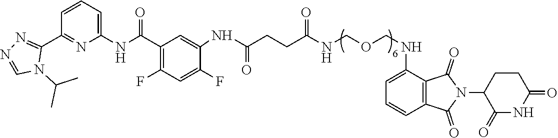 |
| 15 | F | F | 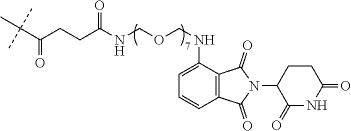 | 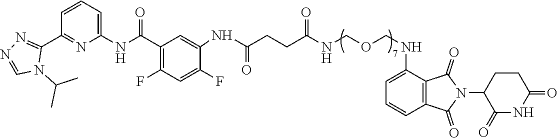 |
| 16 | F | F | 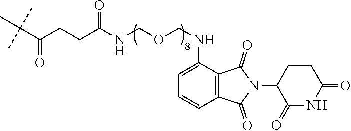 | 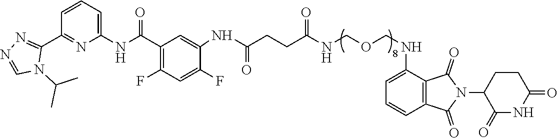 |
| 17 | F | F | 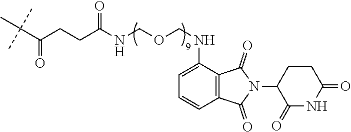 | 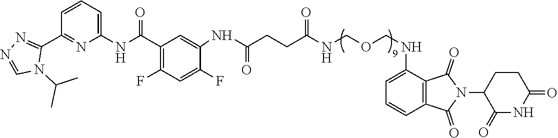 |
| 18 | F | F | 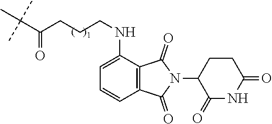 | 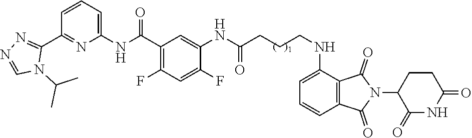 |
| 19 | F | F | 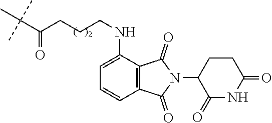 | 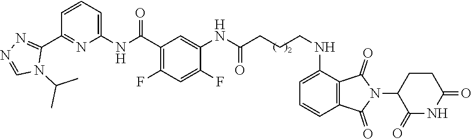 |
| 20 | F | F | 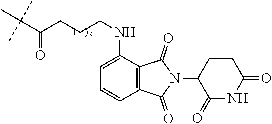 | 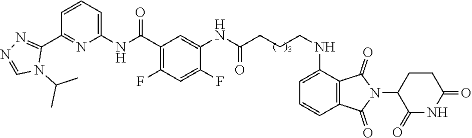 |
| 21 | F | F | 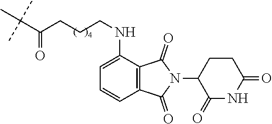 | 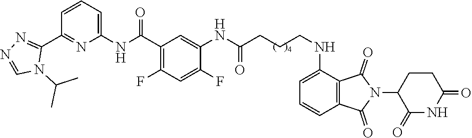 |
| 22 | F | F | 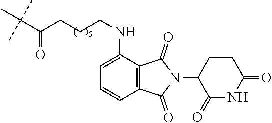 | 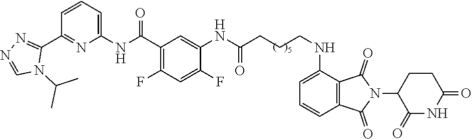 |
| 23 | F | F | 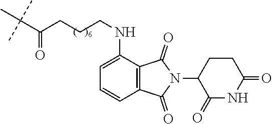 | 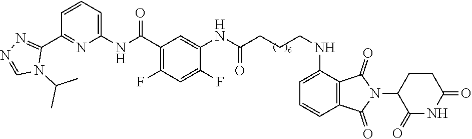 |
| 24 | F | F | 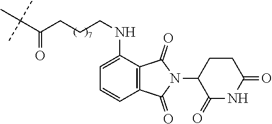 | 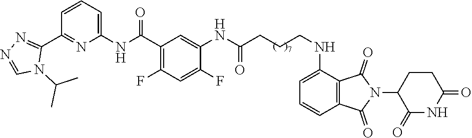 |
| 25 | F | F | 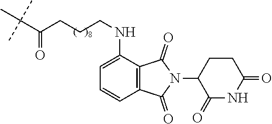 | 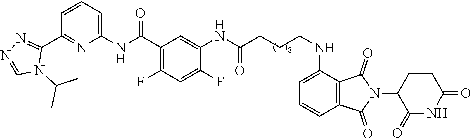 |
| 26 | F | F | 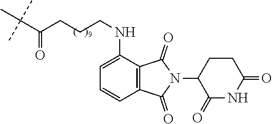 | 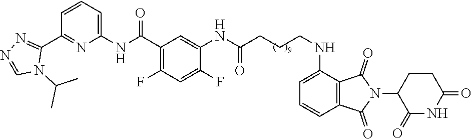 |
| 27 | F | F | 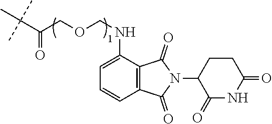 | 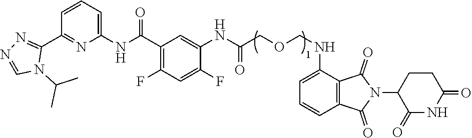 |
| 28 | F | F | 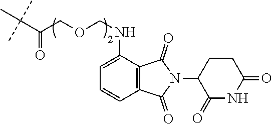 | 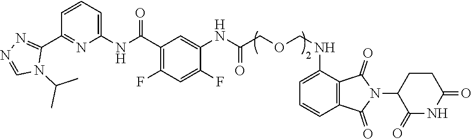 |
| 29 | F | F | 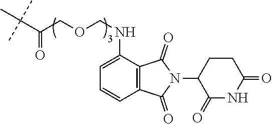 | 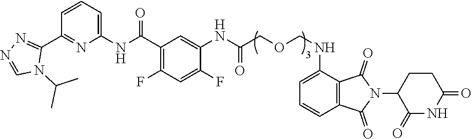 |
| 30 | F | F | 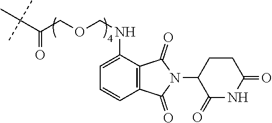 | 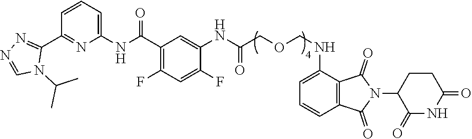 |
| 31 | F | F | 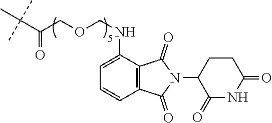 | 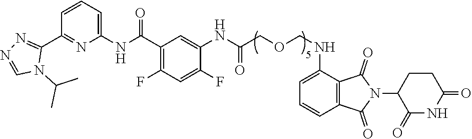 |
| 32 | F | F | 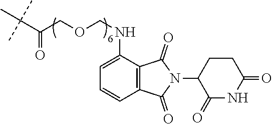 | 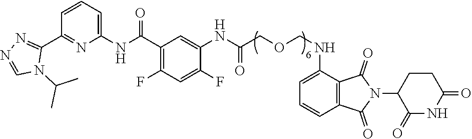 |
| 33 | F | F | 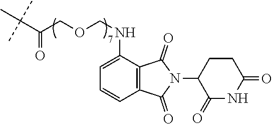 | 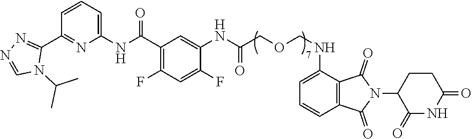 |
| 34 | F | F | 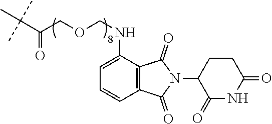 | 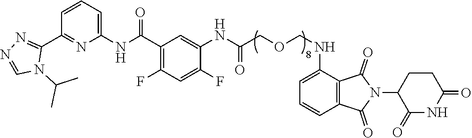 |
| 35 | F | F | 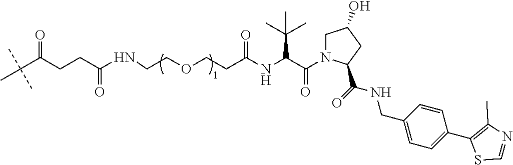 | 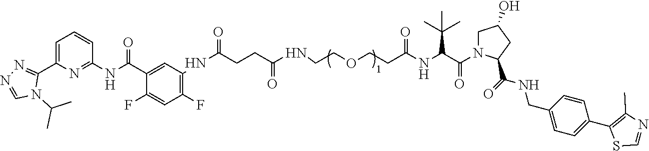 |
| 36 | F | F | 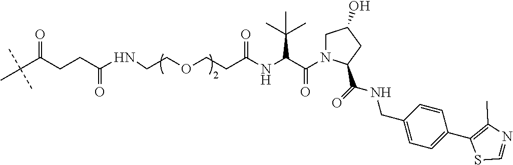 | 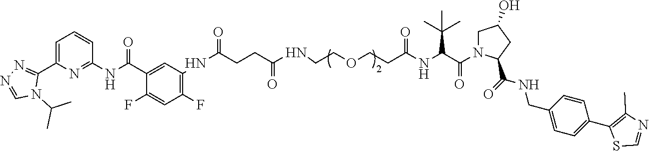 |
| 37 | F | F | 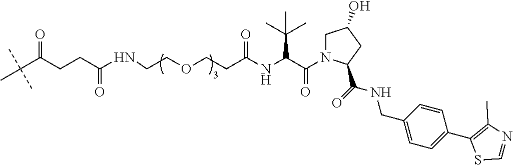 | 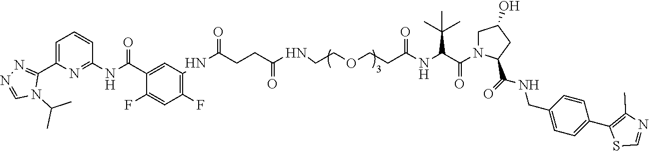 |
| 38 | F | F | 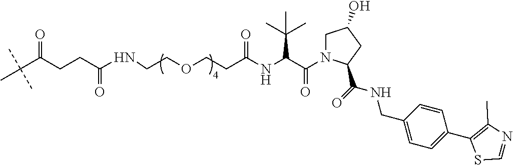 | 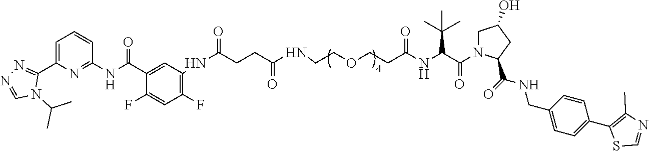 |
| 39 | F | F | 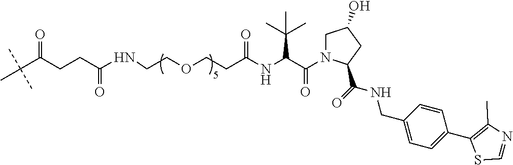 | 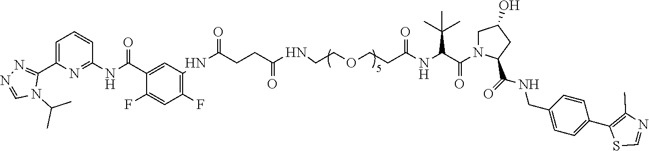 |
| 40 | F | F | 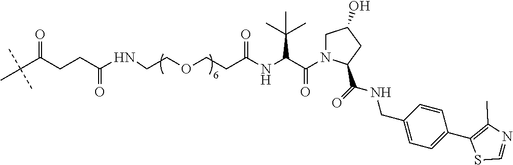 | 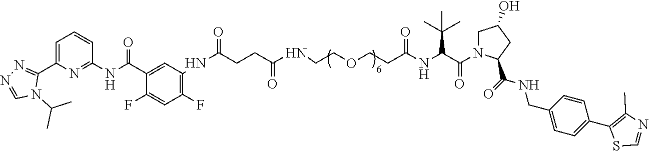 |
| 41 | F | F | 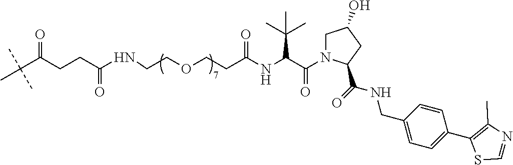 | 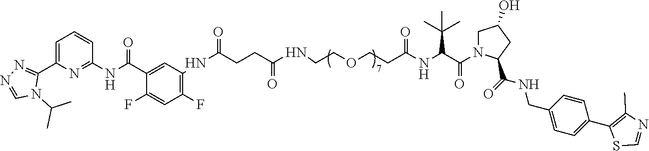 |
| 42 | F | F | 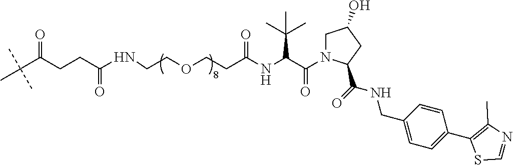 | 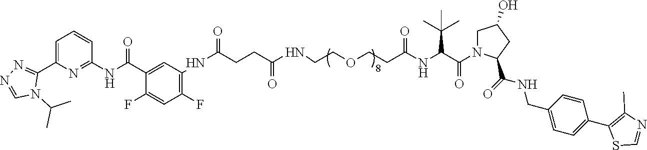 |
| 43 | F | F | 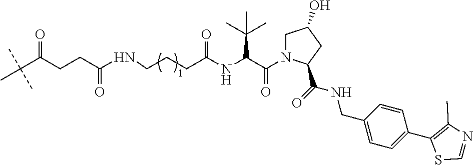 | 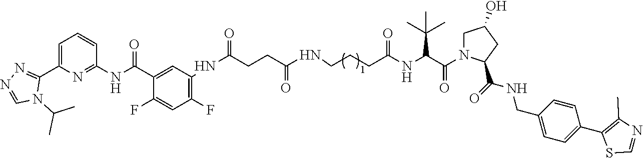 |
| 44 | F | F | 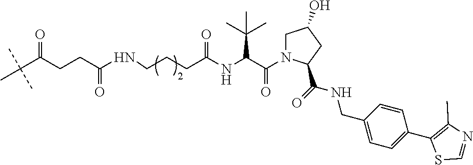 | 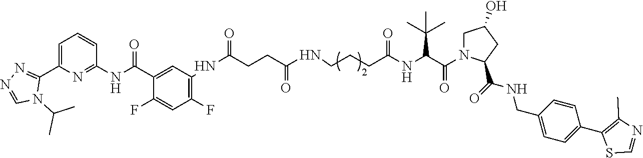 |
| 45 | F | F | 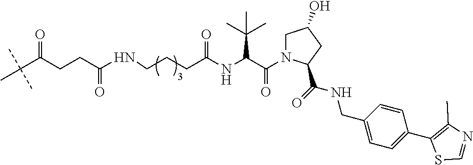 | 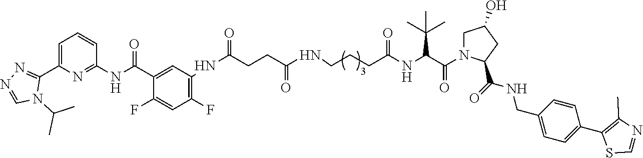 |
| 46 | F | F | 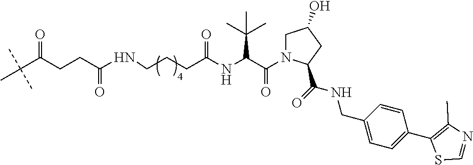 | 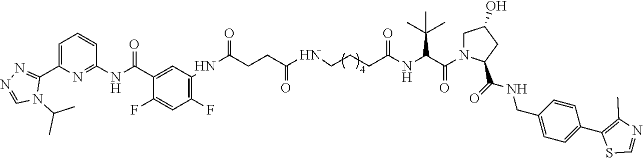 |
| 47 | F | F | 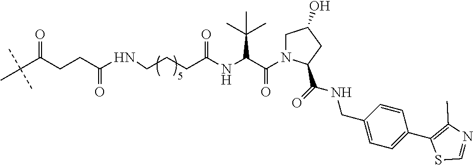 | 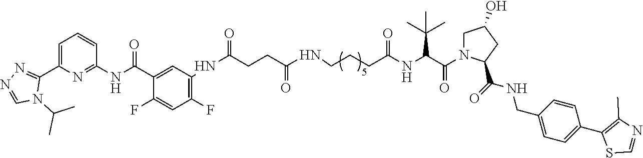 |
| 48 | F | F | 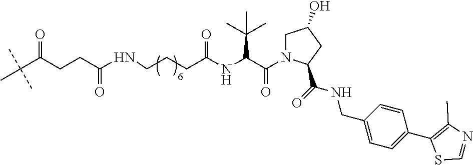 | 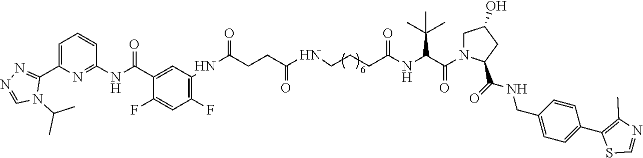 |
| 49 | F | F | 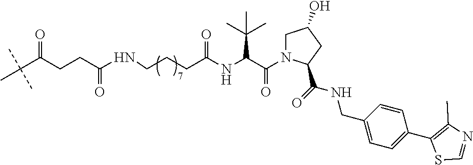 | 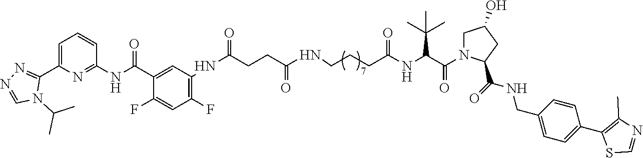 |
| 50 | F | F | 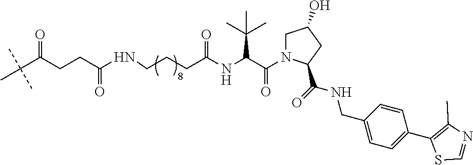 | 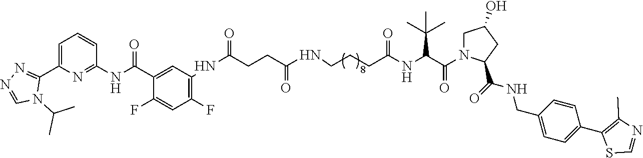 |
| 51 | F | F | 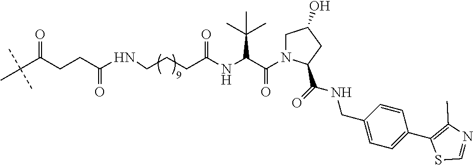 | 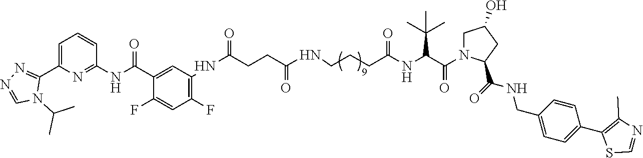 |
| 52 | F | F | 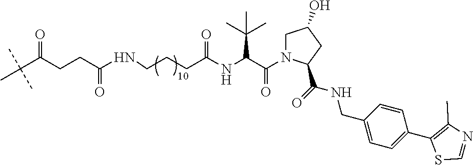 | 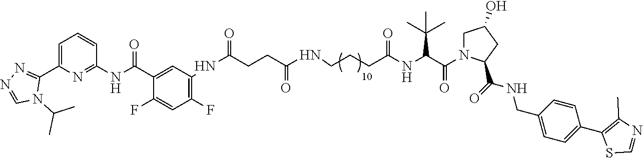 |
| 53 | F | CH₃ | 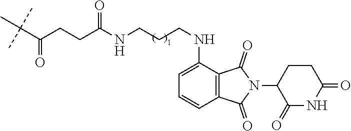 | 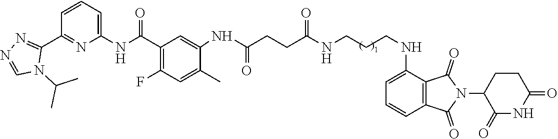 |
| 54 | F | CH₃ | 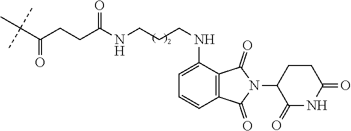 | 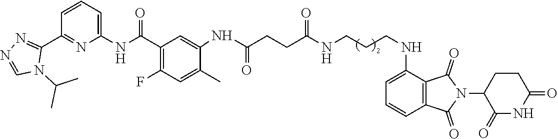 |
| 55 | F | CH₃ | 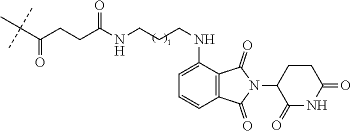 | 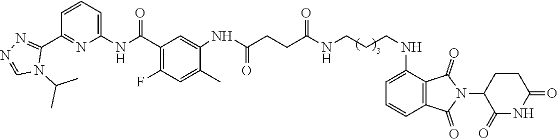 |
| 56 | F | CH₃ | 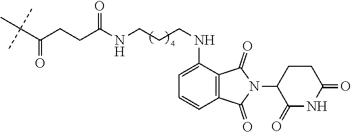 | 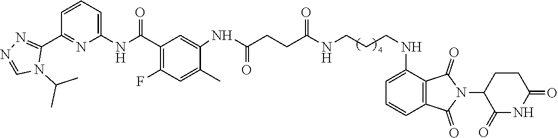 |
| 57 | F | CH₃ | 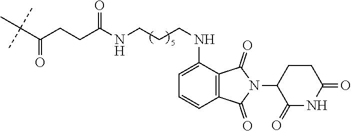 | 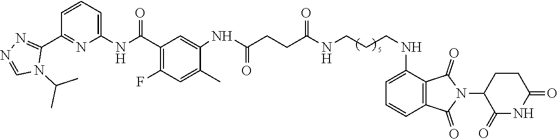 |
| 58 | F | CH₃ | 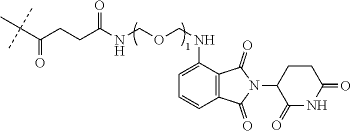 | 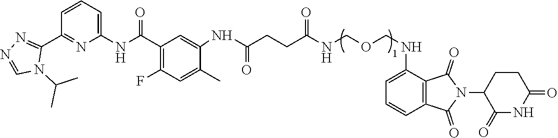 |
| 59 | F | CH₃ | 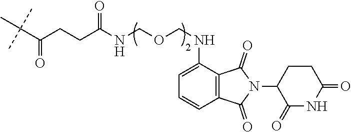 | 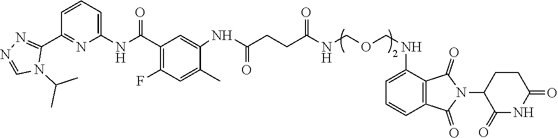 |
| 60 | F | CH₃ | 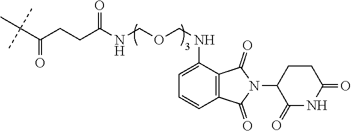 | 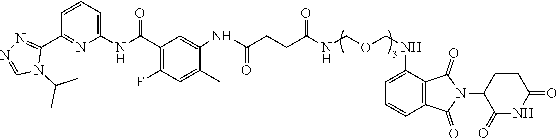 |
| 61 | F | CH₃ | 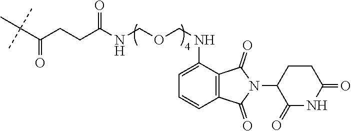 | 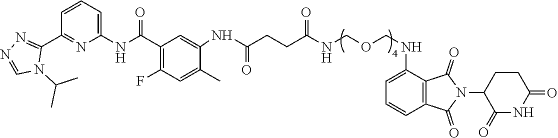 |
| 62 | F | CH₃ | 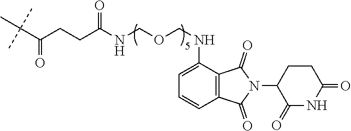 | 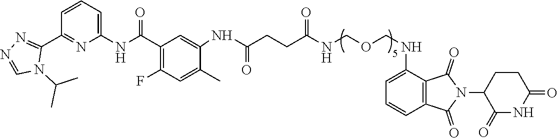 |
| 63 | F | CH₃ | 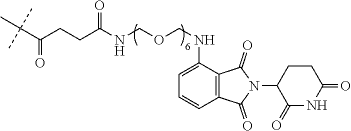 | 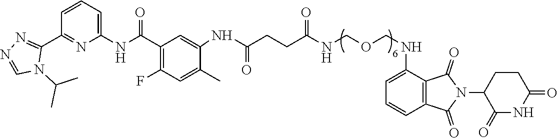 |
| 64 | F | CH₃ | 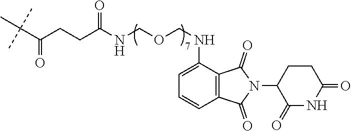 | 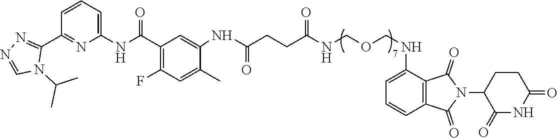 |
| 65 | F | CH₃ | 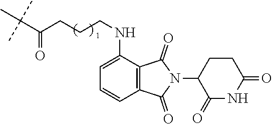 | 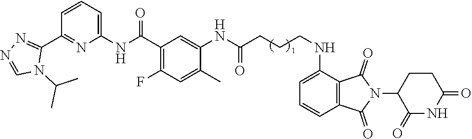 |
| 66 | F | CH₃ | 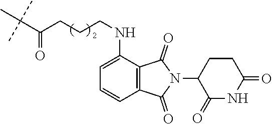 | 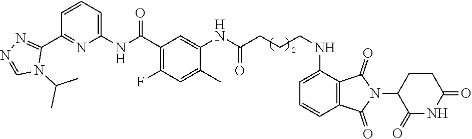 |
| 67 | F | CH₃ | 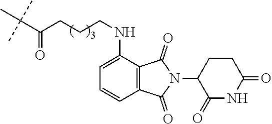 | 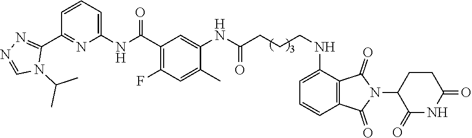 |
| 68 | F | CH₃ | 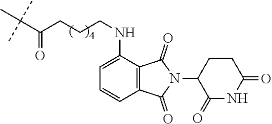 | 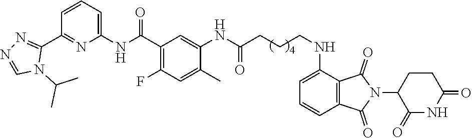 |
| 69 | F | CH₃ | 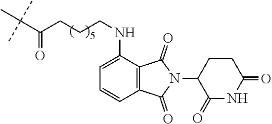 | 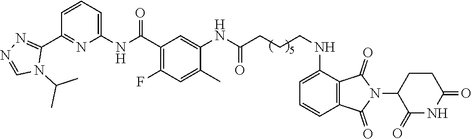 |
| 70 | F | CH₃ | 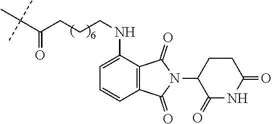 | 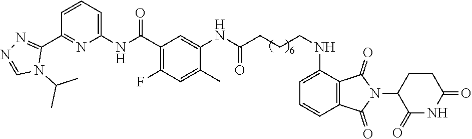 |
| 71 | F | CH₃ | 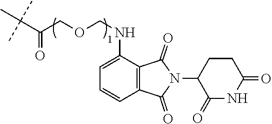 | 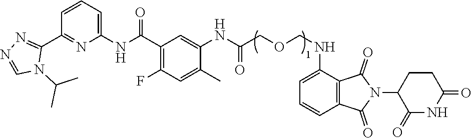 |
| 72 | F | CH₃ | 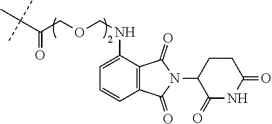 | 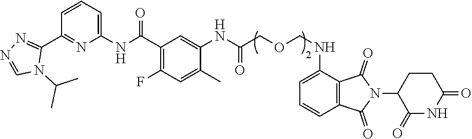 |
| 73 | F | CH₃ | 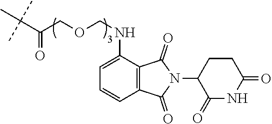 | 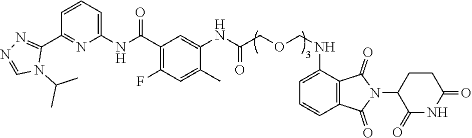 |
| 74 | F | CH₃ | 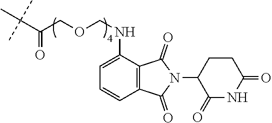 | 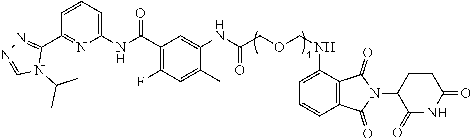 |
| 75 | F | CH₃ | 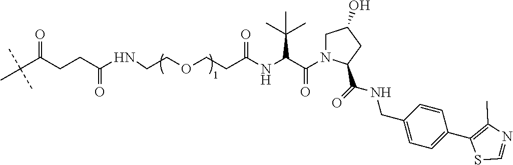 | 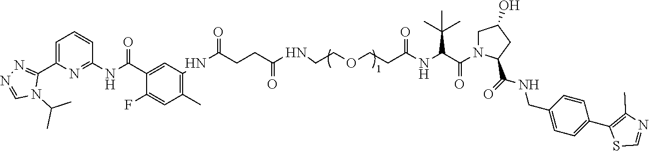 |
| 76 | F | CH₃ | 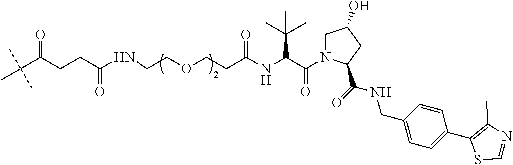 | 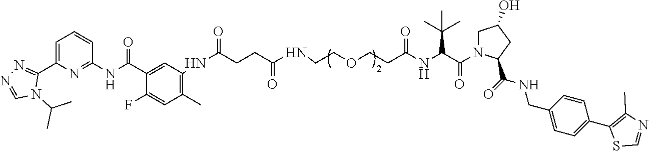 |
| 77 | F | CH₃ | 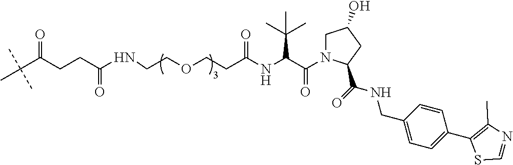 | 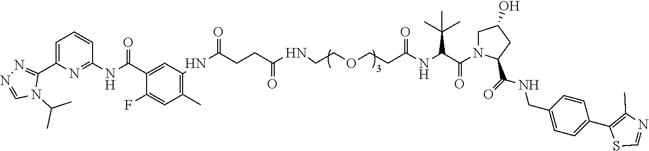 |
| 78 | F | CH₃ | 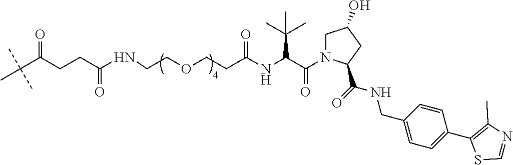 | 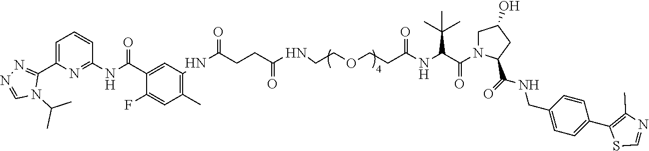 |
| 79 | F | CH₃ | 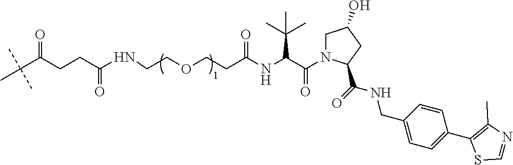 | 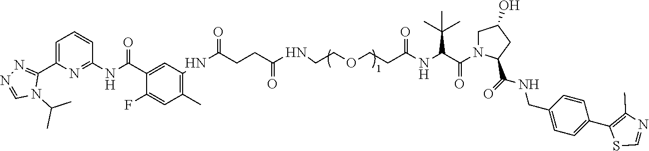 |
| 80 | F | CH₃ | 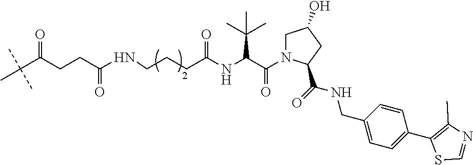 | 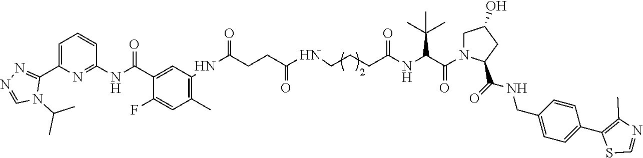 |
| 81 | F | CH₃ | 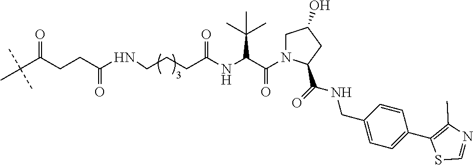 | 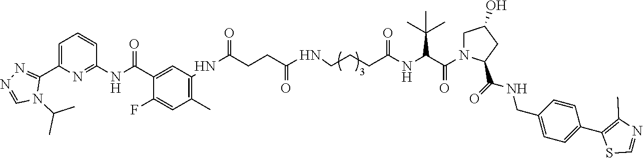 |
| 82 | F | CH₃ | 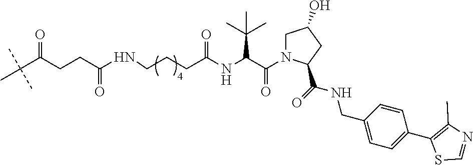 | 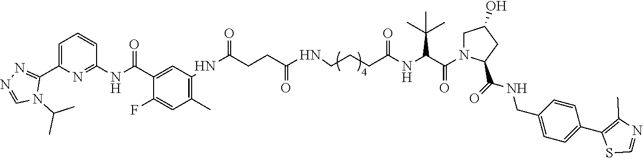 |
| 83 | CH₃ | F | 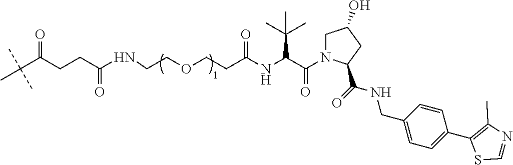 | 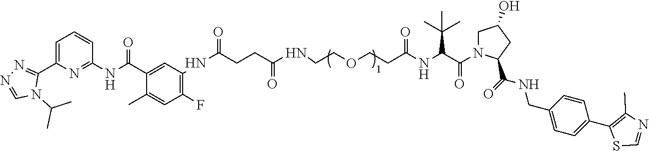 |
| 84 | CH₃ | F | 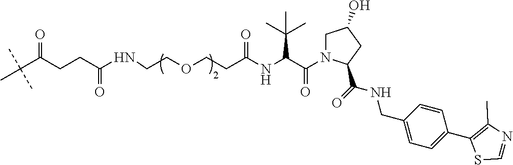 | 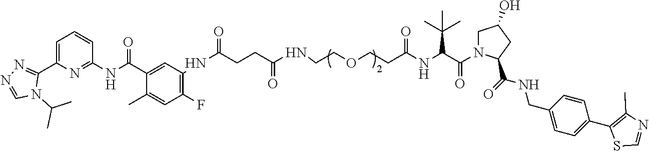 |
| 85 | CH₃ | F | 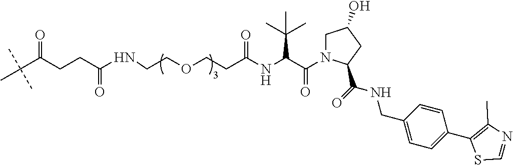 | 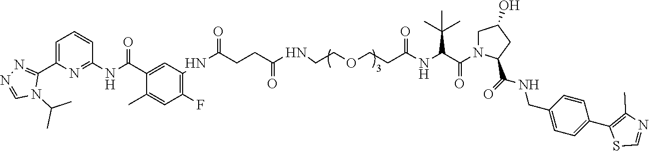 |
| 86 | CH₃ | F | 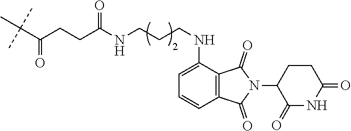 | 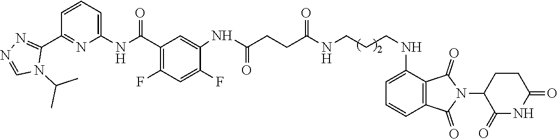 |
| 87 | CH₃ | F | 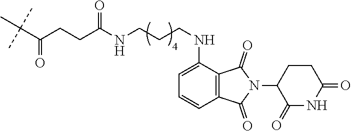 | 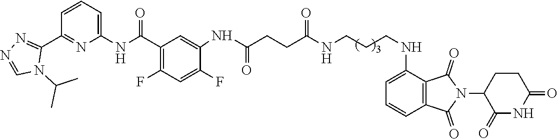 |
| 88 | CH₃ | F | 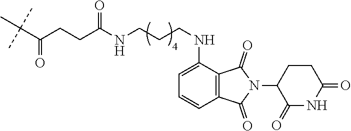 | 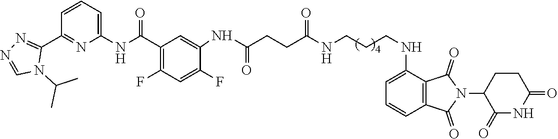 |
| 89 | CH₃ | F | 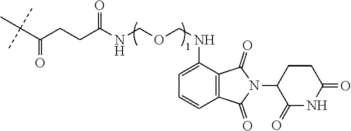 | 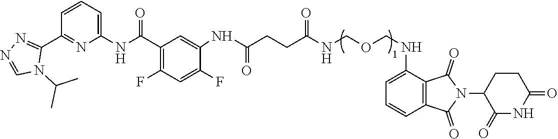 |
| 90 | CH₃ | F | 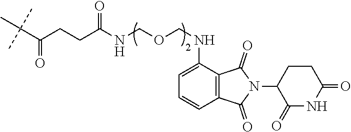 | 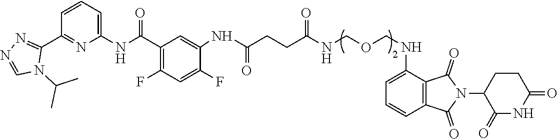 |
| 91 | CH₃ | F | 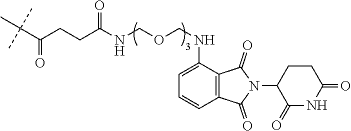 | 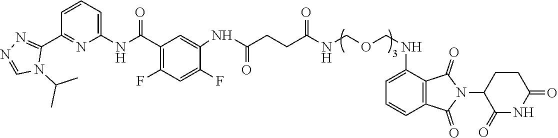 |

### Biological Assay

Several structural modification strategies have been applied to improve the physicochemical properties of PROTACs. For example, basic nitrogen-containing groups such as a pyridine ring are substituted at its C-2 position with a triazole ring or a pyridine ring substituted with 2,6-dione. The groups were further attached by different alkyl and PEG linkers with different chain lengths and/or amide functionality to increase the solubility of PROTACs. Technically, the stability, solubility, and permeability of PROTAC degraders have been determined with the same methods commonly used for small-molecule drugs. For example, in vitro Absorption, Distribution, Metabolism, and Excretion (ADME), drug toxicity, Caco-2 cell permeability assay, and human Ether-a-go-go Related Gene (hERG) assay.

### Example 54

### Cell Culture

Eagle's Minimum Essential Medium (MEM) supplemented with 10% FBS was used to culture HepG2 cells. Aspirate and add fresh culture medium every 2-3 days. HepG2 cell doubling time is 48 hours. To passage cells, rinse the cell monolayer with 1×PBS twice and add pre-warmed (37° C.) 0.05% Trypsin-EDTA solution to cover the bottom of the flask; incubate for 5-7 minutes. As cells detach, neutralize the Trypsin by adding 4× volume of complete growth medium with 10% FBS and gently resuspend the cells by pipetting. To avoid clumping do not agitate the cells by shaking the flask while waiting for detachment. Split cells 1:4 every 3 days. Cultures have been incubated at 37° C. in a CO₂ incubator with 5% CO₂.

### Example 55

Western blotting and immunoblotting: We have assessed the degradation of ASK1 in HepG2 as well as HEK293 cells with our newly synthesized PROTACs in both dose and time-dependent manner. The human HepG2 (liver cancer) and HEK293 (human embryonic kidney) cell lines were obtained from the American Type Culture Collection (ATCC, Manassas, VA). Cells were propagated in MEM medium supplemented with 10% FBS and penicillin (100 U/ml), streptomycin (100 mg/ml), and L-glutamine (0.29 mg/ml) at 37° C. and 5% CO₂. We prepared 10% polyacrylamide gel for SDS-PAGE followed by visualization of the target protein ASK1 (mol. wt. of ASK1: 155 kDa) to the loading control actin (mol. wt. of actin: 42 kDa). Primary antibody anti-ASK1 (1:2500-4000) and anti-Actin (1:2500-6000), and secondary antibody anti-mouse HRP (1:2500-10000) have been used for this purpose. Antibodies against ASK1 (#8662), phospho-ASK1 (#3765), p38 MAP kinase (#8690), phospho-p38 MAP kinase (#4511), JNK (#9252), phospho-JNK (#4668), Cleaved Caspase-3 (CC3) (#9661), Caspase 9 (C9) (#9508), Ubiquitin (#3933), Histone H3 (#4499) and MYC-TAG (#2278) were purchased from Cell Signaling Technology (Beverly, USA). Anti-PEDF was from Millipore (Massachusetts, USA) and 4-Hydroxynoneal (4-HNE) was from R&D Systems (MAB3249, Minneapolis, USA). β-actin (#A2228) and α-tubulin (#T6199) antibodies were purchased from Sigma-Aldrich (St. Louis, Missouri, United States).

HepG2 cells were equally distributed (4-5×105) in 12 well-cell culture plates. The next day the cells were replenished with fresh media and the respective PROTAC treatment was given at a concentration ranging from 1 nM-50 μM for 8 h. After the incubation, the media was aspirated and washed with 1×PBS. The cells were harvested in 80 μl lysis buffer containing 50 mM Tris-HCl (pH 7.4), 100 mM NaCl, 1 mM EDTA, 1 mM EGTA, 1% Triton X-100 and protease inhibitor cocktail (Millipore, MA, USA). The cell lysate suspension was centrifuged at 20,000 g for 20 min and the supernatant containing the protein lysate was collected. Protein in the cell lysate was estimated using the Bradford assay. Bradford's reagent (BioRad) was diluted in a 1:4 ratio in double distilled water. 2 μl of protein sample was added to 100 μl of the reagent and absorbance was measured at 595 nm. 40 μg of protein was diluted in lysis buffer. 1× loading buffer diluted from 5× stock containing 250 mM Tris-HCl (pH 6.8), 10% SDS, 50% glycerol, 0.1% bromophenol Blue and 10% β-mercaptoethanol was added. The protein samples were then heated at 95° C. for 10 minutes, cooled and centrifuged at 10,000 g for 5 minutes before loading. For, western blotting, the proteins were resolved in 8% SDS PAGE in 1× running buffer containing SDS, Tris Base and Glycine. The resolution of the proteins were achieved at 90V. Transfer was done using PVDF membrane (Millipore) having a pore size of 0.45 μm. 1× transfer buffer containing Tris-Base, Glycine and 20% methanol was used for wet transfer. The transfer was done at 90V for 4 hours. Following the transfer, the PVDF membrane containing the proteins was incubated for 1 hour at room temperature in 5% skim milk powder diluted in 1×TBST. After blocking the membrane was washed thoroughly with TBST. Following this, the required primary antibody-like ASK1, JNK, pJNK (Cell Signalling Technology) and Actin (Sigma) was prepared with 1×TBST, 1% Bovine Serum Albumin and 0.04% Sodium Azide was added to the membrane and incubated overnight at 4° C. The next day, the membrane was again washed thoroughly with 1×TBST. The membrane was further incubated with goat anti-rabbit (1:1000-2500) and anti-mouse (1:10000) secondary antibodies for 1 hour at room temperature followed by washing multiple times with 1×TBST. The membrane was then developed using Clarity™ ECL Western Blotting Substrate (BioRad) and viewed in ChemiDoc (Invitrogen).

### Example 56

### ASK1 Kinase Assay

ADP-Glo™ Kinase Assay is a luminescent kinase assay that measures ADP formed from a kinase reaction; ADP is converted into ATP, which is converted into light by Ultra-Glo™ Luciferase. The luminescent signal positively correlates with ADP amount and kinase activity. The assay is well suited for measuring the effects chemical compounds have on the activity of a broad range of purified kinases-making it ideal for both primary screenings as well as kinase selectivity profiling. The ADP-Glo™ Kinase Assay (Promega) was used to monitor the activity of virtually any ADP-generating enzyme (e.g., kinase or ATPase) using up to 1 mM ATP. Diluted enzyme, substrate, ATP and inhibitors in Kinase Buffer. 1 μl of inhibitor or (5% DMSO), 2 μl of the enzyme, and 2 μl of substrate/ATP mixture were added to the wells of a 96-well plate and then incubated at room temperature for 60 minutes. 5 μl of ADP-Glo™ reagent were added and incubated at room temperature for 40 minutes. 10 μl of Kinase Detection Reagent was then added to the reaction mixture and further incubated at room temperature for 30 minutes. Luminescence was recorded (Integration time 0.5-1 second) to get the values of the kinase activity assay.

### Example 57

### Status of Ubiquitination Upon PROTAC Treatment Assessed by Immunoprecipitation Assay

The PROTAC molecule is proposed to efficiently degrade total ASK1 through the proteasome. But proteasomal inhibitor MG132 could block this degradation and lead to the accumulation of total ASK1. Indicating that this degradation is occurring through the proteasomal pathway. After the addition of the active PROTAC molecules, ASK1 gets ubiquitinated and ultimately degraded via the proteasomal-mediated pathway. Thus, the PROTACs are expected to bring about an increase in the ubiquitination levels of ASK1. The compounds of the present invention have shown an increase in ubiquitination levels of ASK1.

To this end, an immunoprecipitation assay was performed wherein HepG2 cells expressing ASK1 were transfected with HA-Ubiquitin and treated with 5 μM of the active PROTACs. MG-132, a proteasomal inhibitor, was added 2-4 hours before harvesting the cells. Immune complexes were pulled down with anti-Myc antibody and immunoblot was done using anti-HA antibody.

### Example 58

### Computational Analysis

The computational study was performed in Schrödinger Maestro v13.1.141, (Release 2022-1, Platform Windows-x64), operated in a Windows 10, 64-bit workstation with Intel® Xeon® Silver 4214R CPU 2×2.40 GHz processors, 128 GB RAM.

The protein structures of 5FQD and 6OYT were downloaded from Protein Data Bank (www.rcsb.org) in .pdb format.

### Protein Preparation:

The downloaded protein structure files were prepared through Protein Preparation Workflow of Schrödinger Maestro at a pH of 7.4, where the bond orders were assigned, disulphide bonds were generated, hydrogen atoms were added, missing loops were filled using PRIME and protonation states of the residues were generated with Epik, at the same pH. Heavy atoms were minimized abiding force-field OPLS2005 up to a RMSD of 0.30 Å.

### Ligand Preparation:

Two-dimensional structures of the thalidomide and selonsertib-derived moieties were drawn in Cambridge Soft ChemBioDraw Ultra v12.0 and imported in Schrödinger Maestro interphase. A built-in protocol of LigPrep in Schrödinger Maestro was employed to prepare the ligands and generate tautomers at a target pH of 7.4 using Ionizer.

Molecular Docking with Induced-Fit:

All water molecules were removed from the prepared protein structures. In the Standard protocol of Induced Fit Docking in Schrödinger Maestro, the prepared ligands (thalidomide and selonsertib-derived moieties) were selected to dock onto the prepared protein structure of 5FQD and 6OYT respectively. To indicate the docking region box, the centroid of the workspace ligand (co-crystallized lenalidomide and selonsertib in the case of 5FQD and 6OYT respectively) was picked. Ring conformations of the ligands were sampled within the energy window of 2.5 Kcal/mol. For Primary Glide docking the side chains were trimmed, based on B-factor, and the van der Waal scaling of relevant receptor and ligand atoms were kept at 0.70 and 0.50 respectively. Through Prime, the residues within 5.0 Å of the ligand poses, were refined, with side chain optimization. Glide SP settings were applied to re-dock the ligands into 20 least-energy induced fit receptor structures, having the lowest Prime energy or within a range of lowest energy plus 30 Kcal/mol. A maximum of 20 structures were generated, which were ranked according to their IFD scores. In the case of selonsertib-derived substructure docked to 6OYT, the structure with IFD score of −2252.73 and docking score of −9.296 was selected as it ranked among the top 5 models and also showed pose-similarity with the co-crystallized ligand. Similarly, in the case of the thalidomide substructure docked to CRBN of 5FQD, the selected structure had IFD and docking scores of −3372.55 and −12.981 respectively and had a pose identical to that of co-crystallized ligand.

### Preparation of the Complex (DDB1-CRBN-ASK1)

From the selected structure of the IFD of selonsertib moiety-6OYT, the ASK1 domain was structurally aligned to the CK1 domain of the selected IFD structure of thalidomide-5FQD. All other protein chains were deleted except the CRBN and the ASK1 with their docked corresponding ligands and the adjacent DDB1. The two protein structures were merged into one file.

### Optimization of the Complex

The merged consolidated complex was minimized using the Prime Minimize application in Schrödinger Maestro, with a VSGB solvation model and an additional distance constraint of 8 Å between the nitrogen atoms of the terminal primary amines of both docked ligands at solvent-exposed interphases of CRBN and ASK1. This step was repeated five times with different distance constraints of 10, 12, 13, 15 and 17 Å leading to the generation of six different protein complexes.

### Manual Modelling of the Linker and Further Energy Minimization

In each of the generated six protein complexes, a PEG linker was manually modelled joining the nitrogen atoms of the terminal amines of two docked ligands, in such a way that it follows the surface topology of the protein. These complexes with the modelled linker were further energy minimized using Prime Minimize application, without any constraints.

### Binding Free Energy Estimation

For understanding which one of these six protein complexes is most stable and best represents the biological binding pattern, free energy for binding (dG Bind) was estimated by Prime MM-GBSA in Schrödinger Maestro with VSGB solvation model.

$$MM - GBSA Δ G Binding = {Energy}_{Complex} - {Energy}_{Receptor} - {Energy}_{Ligand}$$

| Protein complex minimized with an | MM-GBSA ΔG |
|---|---|
| in-between distance of | Binding |
| 8 Å | −117.56 |
| 10 Å | −163.09 |
| 12 Å | −172.34 |
| 13 Å | −167.98 |
| 15 Å | −168.06 |
| 17 Å | −131.07 |

### ADVANTAGES OF THE INVENTION

The various advantage of the present invention is:
1. The triazole-5-yl(pyridine-2-yl)benzamides compounds having structure I are capable of hijacking E3 ligases and the ubiquitin-proteasome system (UPS) leading to selective degradation of the target proteins ASK1.
2. Heterobifunctional molecules made up of ASK1 target protein-binding ligand Selonsertib derivative and an E3 ligase (CRBN, VHL) recruiting ligand connected by a suitable intervening linker for ASK1 degradation.
3. The triazole-5-yl(pyridine-2-yl)benzamides can selectively degrade the target proteins ASK1 by changing different E3 ligase (CRBN, VHL) recruiting ligands.
4. The compounds having structure I can selectively degrade the target proteins ASK1 by different types and different lengths of intervening linkers that connect target protein-binding ligand Selonsertib derivative and different E3 ligase recruiting ligands.
5. The compounds having structure I can exhibit similar phenotypes to those observed in knockdown experiments utilizing genetic tools such as short hairpin RNA (shRNA), small interfering RNA (siRNA) and Clustered Regularly Interspaced Short Palindromic Repeats (CRISPR).
6. The triazole-5-yl(pyridine-2-yl)benzamides compounds having structure I are capable of degrading ASK1 within 1-100 nM, leading to fast, selective, efficient, and prolonged degradation of ASK1 via CRBN E3-ligase in a proteasome-dependent manner in HepG2 and HEK293 cells.
7. The triazole-5-yl(pyridine-2-yl)benzamides compounds having structure I are capable of targeted degradation of ASK1 has potential in patients with hepatic fibrosis and in NAFLD/NASH research where modulation of therapeutic target ASK1 is important.

What is claimed is:

1. A triazole-5-yl(pyridine-2-yl)benzamides compounds having structure I or salts thereof, 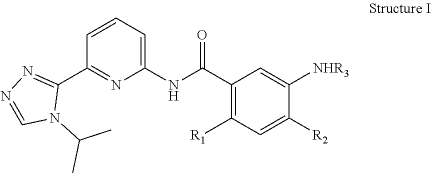
wherein
R₁ is independently selected from groups 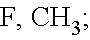
R₂ is independently selected from groups 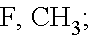
R₃ is independently selected from groups

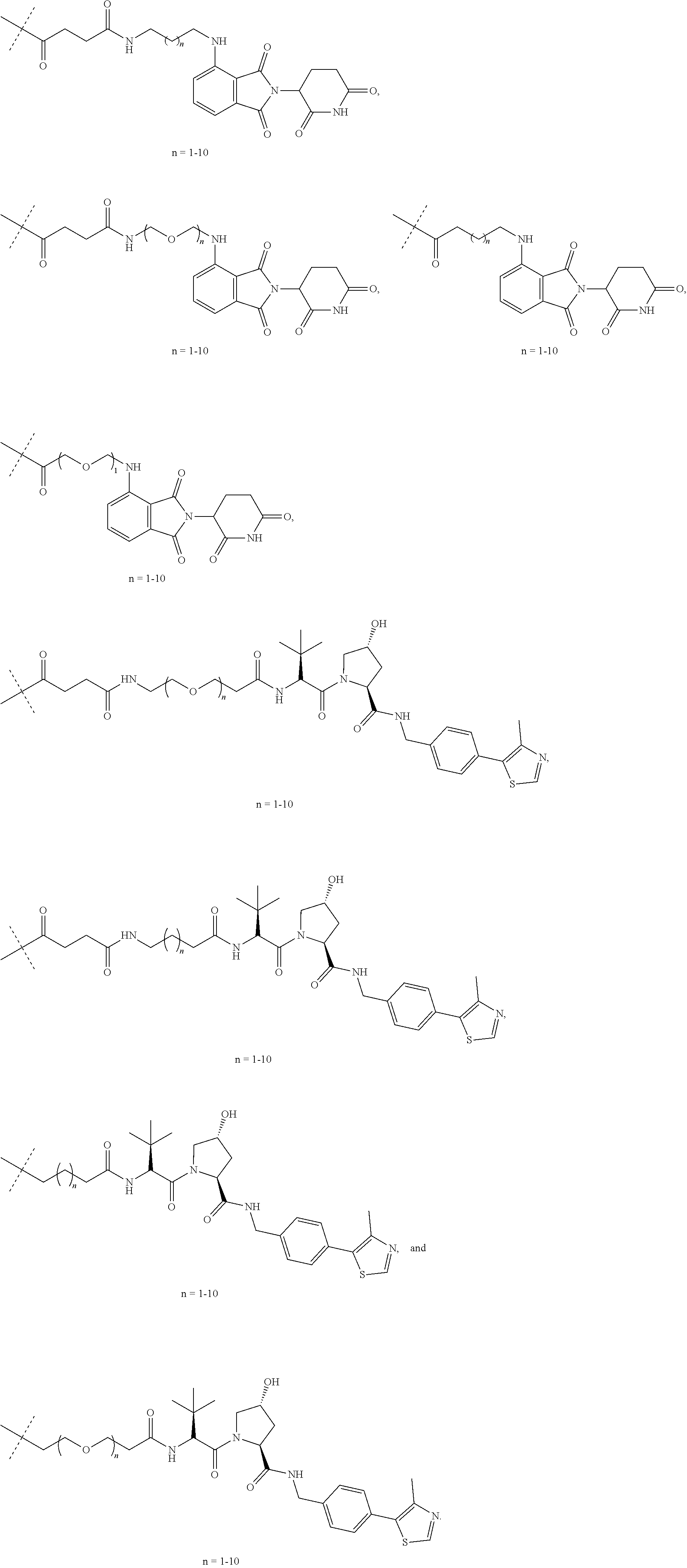

2. The compound as claimed in claim 1, wherein said compound is selected from the group consisting of:
N1-(2,4-difluoro-5-((6-(4-isopropyl-4H-1,2,4-triazol-3-yl)pyridin-2-yl)carbamoyl)phenyl)-N4-(2-(2-((2-(2,6-dioxopiperidin-3-yl)-1,3-dioxoisoindolin-4-yl)amino)ethoxy)ethyl)succinamide (16),
N1-(2,4-difluoro-5-((6-(4-isopropyl-4H-1,2,4-triazol-3-yl)pyridin-2-yl)carbamoyl)phenyl)-N4-(2-(2-(2-((2-(2,6-dioxopiperidin-3-yl)-1,3-dioxoisoindolin-4-yl)amino)ethoxy)ethoxy)ethyl)succinamide (19),
N1-(2,4-difluoro-5-((6-(4-isopropyl-4H-1,2,4-triazol-3-yl)pyridin-2-yl)carbamoyl)phenyl)-N4-(2-(2-(2-(2-((2-(2,6-dioxopiperidin-3-yl)-1,3-dioxoisoindolin-4-yl)amino)ethoxy)ethoxy)ethoxy)ethyl)succinamide (22),
N1-(2,4-difluoro-5-((6-(4-isopropyl-4H-1,2,4-triazol-3-yl)pyridin-2-yl)carbamoyl)phenyl)-N4-(14-((2-(2,6-dioxopiperidin-3-yl)-1,3-dioxoisoindolin-4-yl)amino)-3,6,9,12-tetraoxatetradecyl)succinamide (25),
N1-(2,4-difluoro-5-((6-(4-isopropyl-4H-1,2,4-triazol-3-yl)pyridin-2-yl)carbamoyl)phenyl)-N4-(17-((2-(2,6-dioxopiperidin-3-yl)-1,3-dioxoisoindolin-4-yl)amino)-3,6,9,12,15-pentaoxaheptadecyl)succinamide (28),
N1-(2,4-difluoro-5-((6-(4-isopropyl-4H-1,2,4-triazol-3-yl)pyridin-2-yl)carbamoyl)phenyl)-N4-(20-((2-(2,6-dioxopiperidin-3-yl)-1,3-dioxoisoindolin-4-yl)amino)-3,6,9,12,15,18-hexaoxaicosyl)succinamide (31),
N1-(2,4-difluoro-5-((6-(4-isopropyl-4H-1,2,4-triazol-3-yl)pyridin-2-yl)carbamoyl)phenyl)-N4-(5-((2-(2,6-dioxopiperidin-3-yl)-1,3-dioxoisoindolin-4-yl)amino)pentyl)succinamide (35),
5-(4-((2-(2,6-dioxopiperidin-3-yl)-1,3-dioxoisoindolin-4-yl)amino)butanamido)-2,4-difluoro-N-(6-(4-isopropyl-4H-1,2,4-triazol-3-yl)pyridin-2-yl)benzamide (38),
5-(5-((2-(2,6-dioxopiperidin-3-yl)-1,3-dioxoisoindolin-4-yl)amino)pentanamido)-2,4-difluoro-N-(6-(4-isopropyl-4H-1,2,4-triazol-3-yl)pyridin-2-yl)benzamide (41),
5-(6-((2-(2,6-dioxopiperidin-3-yl)-1,3-dioxoisoindolin-4-yl)amino)hexanamido)-2,4-difluoro-N-(6-(4-isopropyl-4H-1,2,4-triazol-3-yl)pyridin-2-yl)benzamide (44),
5-(7-((2-(2,6-dioxopiperidin-3-yl)-1,3-dioxoisoindolin-4-yl)amino)heptanamido)-2,4-difluoro-N-(6-(4-isopropyl-4H-1,2,4-triazol-3-yl)pyridin-2-yl)benzamide (47),
5-(8-((2-(2,6-dioxopiperidin-3-yl)-1,3-dioxoisoindolin-4-yl)amino)octanamido)-2,4-difluoro-N-(6-(4-isopropyl-4H-1,2,4-triazol-3-yl)pyridin-2-yl)benzamide (50),
5-(9-((2-(2,6-dioxopiperidin-3-yl)-1,3-dioxoisoindolin-4-yl)amino)nonanamido)-2,4-difluoro-N-(6-(4-isopropyl-4H-1,2,4-triazol-3-yl)pyridin-2-yl)benzamide (53),
5-(9-((2-(2,6-dioxopiperidin-3-yl)-1,3-dioxoisoindolin-4-yl)amino)nonanamido)-2,4-difluoro-N-(6-(4-isopropyl-4H-1,2,4-triazol-3-yl)pyridin-2-yl)benzamide (56),
N1-(2,4-difluoro-5-((6-(4-isopropyl-4H-1,2,4-triazol-3-yl)pyridin-2-yl)carbamoyl)phenyl)-N4-(2-(3-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)succinamide (61),
N1-(2,4-difluoro-5-((6-(4-isopropyl-4H-1,2,4-triazol-3-yl)pyridin-2-yl)carbamoyl)phenyl)-N4-(4-((2-(2,6-dioxopiperidin-3-yl)-1,3-dioxoisoindolin-4-yl)amino)butyl)succinamide (65),
N1-(2,4-difluoro-5-((6-(4-isopropyl-4H-1,2,4-triazol-3-yl)pyridin-2-yl)carbamoyl)phenyl)-N4-(6-((2-(2,6-dioxopiperidin-3-yl)-1,3-dioxoisoindolin-4-yl)amino)hexyl)succinamide (68),
N1-(2,4-difluoro-5-((6-(4-isopropyl-4H-1,2,4-triazol-3-yl)pyridin-2-yl)carbamoyl)phenyl)-N4-(7-((2-(2,6-dioxopiperidin-3-yl)-1,3-dioxoisoindolin-4-yl)amino)heptyl)succinamide (72),
N1-(2,4-difluoro-5-((6-(4-isopropyl-4H-1,2,4-triazol-3-yl)pyridin-2-yl)carbamoyl)phenyl)-N4-(8-((2-(2,6-dioxopiperidin-3-yl)-1,3-dioxoisoindolin-4-yl)amino)octyl)succinamide (75),
N1-(2,4-difluoro-5-((6-(4-isopropyl-4H-1,2,4-triazol-3-yl)pyridin-2-yl)carbamoyl)phenyl)-N4-(2-(2-(3-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)ethyl)succinamide (79),
N1-(2,4-difluoro-5-((6-(4-isopropyl-4H-1,2,4-triazol-3-yl)pyridin-2-yl)carbamoyl)phenyl)-N4-((R)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl)succinamide (83),
N1-(2,4-difluoro-5-((6-(4-isopropyl-4H-1,2,4-triazol-3-yl)pyridin-2-yl)carbamoyl)phenyl)-N4-((R)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl)succinamide (87),
N1-(2,4-difluoro-5-((6-(4-isopropyl-4H-1,2,4-triazol-3-yl)pyridin-2-yl)carbamoyl)phenyl)-N4-((R)-20-((2S,4R)-4-hydroxy-2-((4-(4-methylthiazol-5-yl)benzyl)carbamoyl)pyrrolidine-1-carbonyl)-21,21-dimethyl-18-oxo-3,6,9,12,15-pentaoxa-19-azadocosyl)succinamide (91),
N1-(2,4-difluoro-5-((6-(4-isopropyl-4H-1,2,4-triazol-3-yl)pyridin-2-yl)carbamoyl)phenyl)-N4-((R)-23-((2S,4R)-4-hydroxy-2-((4-(4-methylthiazol-5-yl)benzyl)carbamoyl)pyrrolidine-1-carbonyl)-24,24-dimethyl-21-oxo-3,6,9,12,15,18-hexaoxa-22-azapentacosyl)succinamide (95),
N1-(2,4-difluoro-5-((6-(4-isopropyl-4H-1,2,4-triazol-3-yl)pyridin-2-yl)carbamoyl)phenyl)-N4-(8-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctyl)succinamide (99),
N1-(2,4-difluoro-5-((6-(4-isopropyl-4H-1,2,4-triazol-3-yl)pyridin-2-yl)carbamoyl)phenyl)-N4-(8-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctyl)succinamide (103),
N1-(2,4-difluoro-5-((6-(4-isopropyl-4H-1,2,4-triazol-3-yl)pyridin-2-yl)carbamoyl)phenyl)-N4-(11-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecyl)succinamide (107),
N1-(2,4-difluoro-5-((6-(4-isopropyl-4H-1,2,4-triazol-3-yl)pyridin-2-yl)carbamoyl)phenyl)-N4-(14-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-14-oxotetradecyl)succinamide (111),
N1-(2,4-difluoro-5-((6-(4-isopropyl-4H-1,2,4-triazol-3-yl)pyridin-2-yl)carbamoyl)phenyl)-N4-(17-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-17-oxoheptadecyl)succinamide (115), and
N1-(2,4-difluoro-5-((6-(4-isopropyl-4H-1,2,4-triazol-3-yl)pyridin-2-yl)carbamoyl)phenyl)-N4-(20-(((R)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-20-oxoicosyl)succinamide (119).

3. A process for preparation of triazole-5-yl(pyridine-2-yl)benzamides compounds having structure I as claimed in claim 1, comprising the steps of:
taking compound 1 in dichloromethane and acetonitrile (1:1), adding succinic anhydride and a catalytic amount of pyridine, and stirring the reaction mixture at room temperature for 3.5 hours to produce compounds 2a, 2b, 2c, 2d, 2e, and 2f as a light yellowish liquid;
taking compound 3 and compound 4 in acetic acid, adding sodium acetate to the reaction mixture, and setting the reaction mixture to reflux for 12 hours to produce compound 5 as a white solid;
taking compound 6 in methanol, adding hydrazine hydrate to the reaction mixture, and refluxing for 12 hours to produce Compound 7 as a yellowish solid;
taking compound 7 in 1,1-dimethoxy-N,N-dimethylmethanamine, and setting to reflux for 12 hours to produce Compound 8 as a deep brown liquid;
taking compound 8 in a mixture of acetonitrile-acetic acid (4:1), adding Propan-2-1mine to the reaction mixture, and heating under reflux at 100° C. for 12 hours to produce compound 9 as an off-white solid;
taking compound 10 in a dichloromethane, adding oxalyl chloride under the N2 atmosphere and stirring at room temperature for 2 hours, evaporating the solvent to dryness followed by the adding in the mixture compound 6 in pyridine, stirring the final reaction mixture at room temperature for another 2 hours to produce a yellow mass, and purifying the yellow mass by column chromatography using 10% methanol in chloroform to give compound 12 a yellow solid (70% yield);
taking compound 12 in methanol, adding stannous chloride to the reaction mixture with a catalytic amount of HCl, and refluxing the reaction mixture for 3 hours to produce compound 13 as a white powder;
taking compound 13 in N,N-dimethylformamide, adding HATU and N, N-diisopropylethylamine to the reaction mixture, and stirring the reaction mixture at room temperature for overnight to produce compounds 14, 17, 20, 23, 26, 29, 36, 39, 42, 45, 48, 51 and 54 as a colourless liquid;
taking compound 14 in a mixture of 4M HCl in dioxane (1:1), stirring the reaction mixture at room temperature for 2 hours to produce compounds 15, 18, 21, 24, 27, 30, 37, 40, 43, 46, 49, 52, and 55 as a yellowish powder;
taking compound 15 in N,N-dimethylformamide, adding Compound 5, N, N-diisopropylethylamine to the reaction mixture and setting to reflux for 12 hours to produce compounds 16, 19, 22, 25, 28, 31, 38, 41, 44, 47, 50, 53, and 56 as yellow solids;
taking compound 5 in dimethyl sulfoxide (DMSO), adding N, N-diisopropylethylamine to the reaction mixture and setting to reflux for 12 hours to produce Compound 32 as a yellow solid;
taking compound 32 in a mixture of 4M HCl in dioxane in dichloromethane (1:1), and stirring the reaction mixture at room temperature for 2 hours to produce compound 33 as a yellowish powder;
taking compound 33 in dichloromethane and acetonitrile (1:1) & adding succinic anhydride and a catalytic amount of pyridine, and stirring the reaction mixture at room temperature for 3.5 hours to produce compound 34 as a light yellowish liquid;
taking compound 34 in DMF, and adding compound 13, HATU and N, N-Diisopropylethylamine to the reaction mixture, stirring the reaction mixture at room temperature for overnight to produce compound 35 as a yellow solid;
taking compound 57 in N,N-dimethylformamide, adding compound 3-(2-((tert-butoxycarbonyl)amino)ethoxy)propanoic acid, HATU and N, N-diisopropylethylamine to the reaction mixture, and stirring the reaction mixture at room temperature for overnight to produce compound 58 as a colourless liquid;
taking compound 58 in a mixture of 4M HCl in dioxane (1:1), and stirring the reaction mixture at room temperature for 2 hours to produce compound 59 as a white powder;
taking compound 59 in dichloromethane and acetonitrile (1:1), adding succinic anhydride and a catalytic amount of pyridine, and stirring the reaction mixture at room temperature for 3.5 hours to produce compound 60 as a white solid; and
taking compound 60 in N, N-dimethylformamide, and adding compound 13, HATU and N,N-diisopropylethylamine (DIPEA) to the reaction mixture, stirring the reaction mixture at room temperature for overnight to produce compound 61 as a white solid.

4. The compounds as claimed in claim 1, wherein structure I is capable of hijacking E3 ligases and the ubiquitin-proteasome system (UPS) leading to selective degradation of the target protein apoptosis signal-regulating kinase 1 (ASK1).

5. The compounds with structure I such as 16, 41 as claimed in claim 1, wherein structure I is heterobifunctional molecules made up of ASK1 target protein-binding ligand Selonsertib derivative and an E3 ligase (CRBN, VHL) recruiting ligand connected by a suitable intervening linker for ASK1 degradation.

6. The compounds as claimed in claim 1, wherein structure I such as 16, 41 is able to selectively degrade the target proteins ASK1 by changing different E3 ligase (CRBN, VHL) recruiting ligands.

7. The compounds as claimed in claim 1, wherein structure I is able to selectively degrade the target proteins ASK1 by different type (such as PEG and alkyl) and different lengths of intervening linkers that connect target protein-binding ligand Selonsertib derivative and E3 ligase recruiting ligand(s).

8. The compounds as claimed in claim 1, wherein structure I ASK1 within 1-100 nM, leading to fast, selective, efficient, and prolonged degradation of ASK1 via cereblon (CRBN) E3-ligase in a proteasome-dependent manner in both HepG2 and HEK293 cells.

9. The compounds as claimed in claim 1, wherein structure I provides a hypothesis for the structural basis of ASK1 degradation using cereblon (CRBN) by specific PROTAC through computational studies.

10. The compounds as claimed in claim 1, wherein structure I is capable of targeted degradation of ASK1 have potential in patients with hepatic fibrosis and in nonalcoholic fatty liver disease (NAFLD)/nonalcoholic steatohepatitis (NASH) research where modulation of therapeutic target ASK1 is important.
